(19) United States
(12) Patent Application Publication
(10) Pub. No.: US 2008/0293674 A1
(43) Pub. Date: Nov. 27, 2008

(54) Azinyl Imidazoazine and Azinyl Carboxamide

(76) Inventors: Hans-Georg Schwarz, Langenfeld (DE); Jens Frackenpohl, Frankfurt (DE); Achim Hense, Sulzbach (DE); Peter Losel, Leverkusen (DE); Olga Malsam, Rosrath (DE); Karl-Heinz Kuck, Langenfeld (DE); Gerhard Krautstrunk, Monheim (DE); Christian Arnold, Langenfeld (DE)

Correspondence Address: BAYER CROPSCIENCE LP, Patent Department, 2 T .W. ALEXANDER DRIVE, RESEARCH TRIANGLE PARK, NC, 27709, US

(21) Appl. No.: 11/579,703

(22) PCT Filed: Apr. 29, 2005
(86) PCT No.: PCT/EP2005/004616

§ 371 Date: Mar. 14, 2007

(30) Foreign Application Priority Data
May 10, 2004 (DE) 10 2004 022 897.3

(51) Int. Cl. A01N 55/10; C07D 471/04; A01N 43/90; A01P 21/00; A01P 1/00; C07F 7/08

(52) U.S. Cl. 514/63; 546/121; 514/300; 504/246; 504/193; 546/14

(57) ABSTRACT

The invention relates to azinylimidazoazines of structure (I) and their salts and N-oxides,

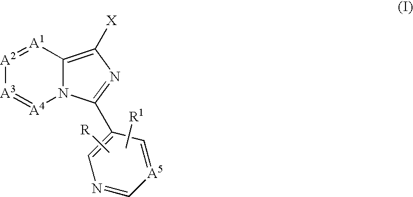

whereby the symbols have the meanings given in the description, as well as to methods for their preparation and new intermediates. The invention further relates to the use of the compounds of structure (I) and the intermediates for the control of zoopests and undesirable micro-organisms.

The invention concerns azinylimidazoazines and their derivatives, methods for their preparation and use as plant protection agents, in particular for the control of zoopests and plant diseases.

The invention also concerns aziny-lcarboxamide intermediates for the preparation of anzinylimidazoazines and the use of these compounds as plant protection agents, in particular for the control of zoopests and plant diseases.

Certain azinyltriazoles, azinyloxadiazoles and azinyloxadiazinones and their possible use as pest control agents, in particular as insecticides, are already known from the (patent) literature (cf. EP-A 185256, WO 01/14373, WO 02/12229). Further azinylcarboxamides with insecticidal activity are known from JP-07010841, JP-07025853 and WO-02/022583.

Since the ecological and economic demands placed on modern plant protection agents are constantly increasing, particularly in respect of toxicity, selectivity, amount applied, residue formation and ease of manufacture, and since problems with, for example, resistance can occur, there is an ongoing demand to develop new plant protection agents which exhibit advantages over known agents, at least in subareas.

It has now been found that new azinylimidazoazines of structure (I) and their salts and N oxides,

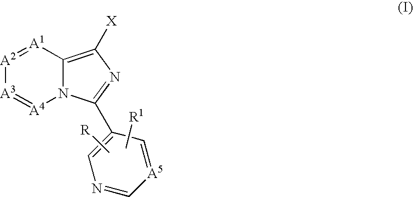

where in structure (I)
A¹, A², A³, A⁴ and A⁵ are the same or different and in each case stand for N (nitrogen) or the group C—R, whereby, however, the imidazoazine bicycle contains in every case 2 to 5 N atoms, and in no case are more than two N atoms next to each other, and whereby R in the group C—R in the individual cases can have in each case the same or different meanings in accordance with the following definition,
R stands in each case for H (hydrogen), nitro, amino, cyano, halogen, or for in each case optionally substituted alkyl, alkoxy, alkylthio, alkylsulphinyl, alkylsulphonyl, alkylamino or dialkylamino, or optionally two neighbouring R groups together stand for alkanediyl or, together with the azine group to which they are connected, form a benzene ring,
R¹ stands for (C₁-C₄-)haloalkyl, and
X stands for H (hydrogen), nitro, formyl, hydroximinomethyl (—CH═N—OH), aminoiminomethyl (—CH═N—NH2), amino, cyano, halogen, or in each case for optionally substituted COOH, aminocarbonyl (—CO—NH₂), alkyl, alkylcarbonyl, alkoxy, alkoxycarbonyl, alkoximinomethyl (—CH═N—O-Alkyl), alkylaminoiminomethyl (CH═N—NH-alkyl), dialkylaminoiminomethyl, cycloalkylalkoxyiminomethyl, benzyloxyiminomethyl, akenyloxyiminomethyl, arylsulphonylaminoiminomethyl, alkylcarbonyloxyiminomethyl, alkylthio, alkylsulphinyl, alkylsulphonyl, alkylamino, aminocarbonyl, hydroxycarbonyl alkylaminocarbonyl, alkenylaminocarbonyl, alkynylaminocarbonyl, dialkylamino, dialkylaminocarbonyl, alkylcarbonylaminocarbonyl, N-alkyl-alkylcarbonylaminocarbonyl, alkoxycarbonylaminocarbonyl, N-alkyl-alkoxycarbonylaminocarbonyl, alkylaminocarbonylaminocarbonyl, N-alkyl-N-alkylaminocarbonylaminocarbonyl, alkenyl, alkenyloxy, alkenylamino, alkenyloximinomethyl, alkynyl, alkynyloxy, alkynylamino, cycloalkyl, cycloalkyloxy, cycloalkylatkoximinomethyl, cycloalkylamino, cycloalkylalkyl, cycloalkylalkoxy, cycloalkylalkylamino, aryl, aryloxy, arylthio, arylamino, arylaminoiminomethyl, arylalkyl, arylethynyl, arylalkoxy, arylalkylthio, arylalkylamino, arylalkylaminoiminomethyl, arylalkoxyiminomethyl, arylsulphonylaminoiminomethyl, heterocyclyl, heterocyclyloxy, heterocyclylthio, heterocyclylamino, heterocyclylalkyl, heterocyclylalkynyl, heterocyclylalkoxy, heterocyclylalkylthio or heterocyclylalkylamino.

Preferred substituents or preferred ranges of the residues present in the formula shown above and listed below are defined in the following.
A¹, A², A³, A⁴ and A⁵ are the same or different and stand in each case preferably for N (nitrogen) or the group C—R, whereby, however, the imidazoazine bicycle contains 2 to 5 nitrogen atoms and in no case are more than two N atoms next to each other and were R in the group C—R in the individual cases can in each case have the same or different meanings in accordance with the following definitions.
R stands in each case for preferably H (hydrogen), nitro, amino, cyano, halogen, or for alkyl, alkoxy, alkylthio, alkylsulphinyl, alkylsulphonyl, alkylamino or dialkylamino with in each case 1 to 6 carbon atoms in the alkyl group, in each case optionally substituted by cyano, halogen or C₁-C₄-alkoxy, or optionally two neighbouring R groups together stand for alkanediyl with 3 to 5 carbon atoms, or optionally two neighbouring R groups, together with the azine group to which they are connected, form a benzene ring.
R¹ stands preferably for CF₃, CHF₂ or CF₂Cl.
X stands preferably for H (hydrogen), hydroxycarbonyl (COOH), nitro, formyl, hydroximinomethyl (—CH═N—OH), aminoiminomethyl (—CH═N—NH2), amino, cyano, halogen, for alkyl with 1 to 6 carbon atoms, optionally substituted by cyano, hydroxy, halogen, C₁-C₄-alkoxy, C₁-C₄-alkylamino, di(C₁-C₄-alkyl)-aminocarbonyloxy, C₁-C₄-alkylcarbonyloxy, benzylamino, dibenzylamino, pyrrolidinyl, piperidinyl (which is optionally substituted by C₁-C₄-haloalkyl), morpholinyl (which is optionally substituted by C₁-C₄-alkyl)piperazinyl, N-methylpiperazinyl or di(C₁-C₄-alkyl)-amino, for aminocarbonyl optionally substituted by benzyloxycarbonyl or N,O-di(C₁-C₄-alkyl)hydroxylaminocarbonyl, for alkylcarbonyl, alkoxy, alkoxycarbonyl, alkoximinomethyl (—CH═N—O-alkyl), alkylaminoiminomethyl (—CH═N—NH-alkyl), dialkylaminoiminomethyl, benzyloxyiminomethyl, C₂-C₈-alkenyloxyiminomethyl, phenylsulphonylaminoiminomethyl, alkylcarbonyloxyiminomethyl, alkylthio, alkylsulphinyl, alkylsulphonyl, alkylamino, alkylaminocarbonyl, dialkylamino, dialkylaminocarbonyl, alkylcarbonylaminocarbonyl, N-alkyl-alkylcarbonylaminocarbonyl, alkoxycarbonylaminocarbonyl, N-alkyl-alkoxycarbonylaminocarbonyl, alkylaminocarbonylaminocarbonyl or N-alkyl-N-alkylaminocarbonylaminocarbonyl with in each case 1 to 6 carbon atoms in the alkyl groups, in each case optionally substituted by cyano, hydroxy, halogen, C₁-C₄-alkoxy, C₁-C₄-alkoxycarbonyl, benzyloxycarbonyl or N,O-dialkylhydroxylaminocarbonyl, for alkenyl, alkenyloxy, alkenylamino, alkenylaminocarbonyl, alkenyloximinomethyl, alkynyl, alkynyloxy, alkynylaminocarbonyl or alkynylamino with in each case 2 to 8 carbon atoms in the alkenyl or alkynyl groups, optionally substituted by cyano, hydroxy, C₁-C₆-alkoxy, phenyl (which itself is optionally substituted by nitro, amino, hydroxy, cyano, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulphinyl, C₁-C₄-haloalkylsulphinyl, C₁-C₄-alkylsulphonyl, C₁-C₄-haloalkylsulphonyl, C₁-C₄-alkylamino, di(C₁-C₄-alkyl)-amino, di(C₁-C₄-alkyl)aminocarbonyl, di(C₁-C₄-alkyl)aminosulphonyl), phenoxy, heterocyclyl (with in each case up to 8 carbon atoms and at least one heteroatom from the series N (nitrogen), O (oxygen), S (sulphur) and optionally with a group CO, CS, SO or SO₂ as component of the heterocycle, which itself is optionally substituted by halogen or C₁-C₄-alkyl), C₁-C₄-alkoxycarbonyl, benzyloxycarbonyl, N,O-di(C₁-C₄-alkyl)aminocarbonyl, trialkylsilyl or halogen, for cycloalkyl, cycloalkyloxy, cycloalkylalkoximinomethyl, cycloalkylamino, cycloalkylalkyl, cycloalkylalkoxy or cycloalkylalkylamino with in each case 3 to 6 carbon atoms in the cycloalkyl group and optionally with 1 to 4 carbon atoms in the alkyl parts, in each case optionally substituted by cyano, halogen, C₁-C₄-alkyl or C₁-C₄-haloalkyl, for aryl, aryloxy, arylthio, arylamino, arylaminoiminomethyl, arylalkyl, arylethynyl, arylalkoxy, arylalkylthio, arylalkylamino, arylalkylaminoiminomethyl, arylalkoxyiminomethyl or arylsulphonylaminoiminomethyl with in each case 6 or 10 carbon atoms in the aryl group and optionally 1 to 4 carbon atoms in the alkyl part, in each case optionally substituted by nitro, amino, hydroxy, cyano, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulphinyl, C₁-C₄-halogenalkylsulphinyl, C₁-C₄-alkylsulphonyl, C₁-C₄-haloalkylsulphonyl, C₁-C₄-alkylamino, di(C₁-C₄-alkyl)-amino, di(C₁-C₄-alkyl)aminocarbonyl, di(C₁-C₄-alkyl)-amino-sulphonyl, phenoxy or phenyl, or for heterocyclyl, heterocyclyloxy, heterocyclylthio, heterocyclylamino, heterocyclylalkyl, heterocyclylalkynyl, heterocyclylalkoxy, heterocyclylalkylthio or heterocyclylalkylamino with in each case up to 8 carbon atoms and at least one heteroatom from the series N (nitrogen), O (oxygen), S (sulphur) and optionally also a group CO, CS, SO or SO₂ as component of the heterocycle as well as optionally up to 4 carbon atoms in the alkyl or alkynyl part, optionally substituted by in each case nitro, amino, hydroxy, cyano, carbamoyl, thiocarbamoyl, halogen, C₁-C₄-alkyl, C₁-C₄-haloalkyl, C₁-C₄-alkoxy, C₁-C₄-halooalkoxy, C₁-C₄-alkylthio, C₁-C₄-alkylthio-C₁-C₄-alkyl, C₁-C₄-haloalkylthio, C₁-C₄-alkylsulphinyl, C₁-C₄-halogenalkylsulphinyl, C₁-C₄-alkylsulphonyl, C₁-C₄-halogenalkyl-sulphonyl, C₁-C₄-alkylamino, di(C₁-C₄-alkyl)amino, di(C₁-C₄-alkyl)aminocarbonyl, di(C₁-C₄-alkyl)aminosulphonyl, benzyl, thienylsulphonylmethyl, piperidinomethyl or phenyl, or for 2,4-dioxaspiro[5.5]undec-8-en-3-yl or 2,4-dioxaspiro[5.5]undecan-3-yl
A¹, A², A³, A⁴ and A⁵ are the same or different and in each case stand more preferably for N (nitrogen) or the group C—R, whereby the imidazoazine bicycle contains 2 to 4 N atoms and whereby R in the group C—R in the individual case can have in each case the same or different meanings in accordance with the following definition.
R stands more preferably for H (hydrogen), nitro, amino, cyano, fluorine, chlorine, bromine, iodine or for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, methylamino, ethylamino, n- or i-propylamino, n-, i-, s- or t-butylamino, dimethylamino or diethylamino, in each case optionally substituted by cyano, fluorine, chlorine, bromine, methoxy, ethoxy, n- or i-propoxy, n-, i-, s- or t-butoxy, or optionally two neighbouring R groups together stand for propane-1,3-diyl, butane-1,3-diyl, butane-1,4-diyl, pentane-1,3-diyl, pentane-1,4-diyl or pentane-1,5-diyl, or optionally two neighbouring R groups, together with the azine group to which they are connected, form a benzene ring.
R¹ stands more preferably for CF₃.
X stands more preferably for H (hydrogen), hydroxycarbonyl (COOH), for aminocarbonyl optionally substituted by benzyloxycarbonyl, N,O-dimethylhydroxylaminocarbonyl, N,O-diethylhydroxylaminocarbonyl, N-methyl-O-ethylhydroxylaminocarbonyl or N-ethyl-O-methylhydroxylaminocarbonyl, for nitro, formyl, hydroximinomethyl (—CH═N—OH), aminoiminomethyl (—CH═N—NH2), amino, cyano, fluorine, chlorine, bromine, iodine, for methyl, ethyl, n- or i-propyl, n-, i-, s- or t-butyl, n-, i-, s-, t- or neo-pentyl in each case optionally substituted by cyano, hydroxy, fluorine, chlorine, bromine, methoxy, ethoxy, n- or i-propoxy, n-, i-, s- or t-butoxy, benzyloxy, methylamino, ethylamino, n- or i-propylamino, n-, i-, s- or t-butylamino, dimethylaminocarbonyloxy, diethylaminocarbonyloxy, methylcarbonyloxy, ethylcarbonyloxy, benzylamino, dibenzylamino, dimethylamino, diethylamino or dipropylamino, for methyl substituted by the group NR′R″ (whereby R′R″ together with the nitrogen atom stands for pyrrolidine, piperidine, 4-trifluoromethylpiperidine, 3-trifluoromethylpiperidine, fluoromethylpiperidine, morpholine, dimethylmorpholine, piperazine or N-methylpiperazine), for acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, methoximinomethyl, ethoximinomethyl, methylaminoiminomethyl, ethylaminoiminomethyl, n- or i-propylaminoiminomethyl, dimethylaminoiminomethyl, cyclohexylmethoxyiminomethyl, cycloentylmethoxyiminomethyl, cyclopropylmethoxyiminomethyl, benzyloxyiminomethyl, chlorobenzyloxyiminomethyl, ethylcarbonyloxyiminomethyl, methylcarbonyloxyiminomethyl, allyloxyiminomethyl, phenylsulphonylaminoiminomethyl, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s- or t-butylamino, methylaminocarbonyl, ethylaminocarbonyl, n- or i-propylaminocarbonyl, dimethylamino, diethylamino, di-n-propylamino, di-i-propylamino, dimethylaminocarbonyl, diethylaminocarbonyl, acetylaminocarbonyl, propionylaminocarbonyl, n- or i-butyroylaminocarbonyl, N-methyl-acetylaminocarbonyl, N-methyl-propionylaminocarbonyl, methoxycarbonylaminocarbonyl, ethoxycarbonylaminocarbonyl, n- or i-propoxycarbonylaminocarbonyl, N-methyl-methoxycarbonylaminocarbonyl, N-methylethoxycarbonylaminocarbonyl, methylaminocarbonylaminocarbonyl, ethylaminocarbonylaminocarbonyl, n- or i-propylamino-carbonylaminocarbonyl, N-methylmethylaminocarbonylaminocarbonyl, N-methyl-ethylaminocarbonylamino, N,O-dimethylhydroxylaminocarbonyl, N,O-diethylhydroxylaminocarbonyl, N-methyl-O-ethylhydroxylaminocarbonyl, N-ethyl-O-methylhydroxylaminocarbonyl, in each case optionally substituted by cyano, hydroxy, fluorine, chlorine, bromine, methoxy, ethoxy, n- or i-propoxy, n-, i-, s- or t-butoxy, for ethenyl, propenyl, butenyl, pentenyl, hexenyl, propenyloxy, butenyloxy, pentenyloxy, propenylamino, butenylamino, pentenylamino, allyloximinomethyl, ethynyl, propynyl, butynyl, pentynyl, hexynyl, heptynyl, propynyloxy, butynyloxy, pentynyloxy, propynylaminocarbonyl, butynylaminocarbonyl or pentynylaminocarbonyl, in each case optionally substituted by cyano, hydroxy, methoxy, ethoxy, n- or i-propoxy, n-, i-, s- or t-butoxy, phenyl (which itself is optionally substituted by C₁-C₄-alkyl, C₁-C₄-alkoxy, halogen, C₁-C₄ haloalkyl), phenoxy, heterocyclyl (selected from furyl, tetrahydrofuryl, thienyl, pyrrolyl, pyrrolinyl, pyrrolidinyl, pyrazolyl, pyrazolinyl, oxazolyl, oxazolinyl, isoxazolyl, isoxazolinyl, thiazolyl, isothiazolyl, oxadiazolyl, thiadiazolyl, pyridinyl, pyrrolidinyl, morpholinyl, piperazinyl or pyrimidinyl, which are optionally substituted by halogen or C₁-C₄-alkyl), trialkylsilyl, ethoxycarbonyl, methoxycarbonyl, fluorine, chlorine or bromine, for cyclopropyl, cyclobutyl, cyclopentyl, cyclohexyl, cyclopropyloxy, cyclobutyloxy, cyclopentyloxy, cyclohexyloxy, cyclopropylamino, cyclobutylamino, cyclopentylamino, cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, cyclopropylmethoximinomethyl, cyclopropylmethylamino, cyclobutylmethylamino, cyclopentylmethylamino or cyclohexylmethylamino optionally substituted by cyano, fluorine, chlorine, bromine, iodine, methyl, ethyl, n- or i-propyl, n-, i-, s- or t-butyl, fluoromethyl, chloromethyl, difluoromethyl, dichloromethyl, trifluoromethyl or trichloromethyl, for phenyl, naphthyl, phenoxy, naphthyloxy, phenylthio, naphthylthio, phenylamino, naphthylamino, phenylaminoiminomethyl, benzyl, phenylethyl, phenylpropyl, phenylethynyl, phenylmethoxy, phenylethoxy, phenylpropoxy, phenylmethylthio, phenylmethylamino, phenylethylamino, phenylmethylaminoiminomethyl, phenylmethoxyiminomethyl or phenylsulphonylaminoiminomethyl, in each case optionally substituted by nitro, amino, hydroxy, cyano, carbamoyl, thiocarbamoyl, fluorine, chlorine, bromine, iodine, methyl, ethyl, n- or i-propyl, n-, i-, s- or t-butyl, fluoromethyl, chloromethyl, difluoromethyl, dichloromethyl, trifluoromethyl, trichloromethyl, fluorodichloromethyl, chlorodifluoromethyl, methoxy, ethoxy, n- or i-propoxy, n-, i-, s- or t-butoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, n-, i-, s- or t-butylthio, difluoromethylthio, trifluoromethylthio, chlorodifluoromethylthio, methylsulphinyl, ethylsulphinyl, n- or i-propylsulphinyl, trifluoromethylsulphinyl, methylsulphonyl, ethylsulphonyl, trifluoromethylsulphonyl, methylamino, ethylamino, n- or i-propylamino, n-, i-, s- or t-butylamino, dimethylamino, diethylamino, dimethylaminocarbonyl, diethylaminocarbonyl, dimethylaminosulphonyl, diethylaminosulphonyl, phenoxy or phenyl, for heterocyclyl, heterocyclyloxy, heterocyclylthio, heterocyclylamino, heterocyclylmethyl, heterocyclylethynyl, heterocyclylmethoxy, heterocyclylmethylthio or heterocyclylmethylamino, in each case optionally substituted by nitro, amino, hydroxy, cyano, carbamoyl, thiocarbamoyl, fluorine, chlorine, bromine, iodine, methyl, ethyl, n- or i-propyl, n-, i-, s- or t-butyl, fluoromethyl, chloromethyl, difluoromethyl, dichloromethyl, trifluoromethyl, trichloromethyl, fluorodichloromethyl, chlorodifluoromethyl, methoxy, ethoxy, n- or i-propoxy, n-, i-, s- or t-butoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, n-, i-, s- or t-butylthio, methylthiomethyl, ethylthiomethyl, difluoromethylthio, trifluoromethylthio, chlorodifluoromethylthio, methylsulphinyl, ethylsulphinyl, n- or i-propylsulphinyl, trifluoromethylsulphinyl, methylsulphonyl, ethylsulphonyl, trifluoromethylsulphonyl, methylamino, ethylamino, n- or i-propylamino, n-, i-, s- or t-butylamino, dimethylamino, diethylamino, dimethylaminocarbonyl, diethylaminocarbonyl, dimethylaminosulphonyl, diethylaminosulphonyl, thienylsulphonylmethyl, piperidinomethyl, benzyl or phenyl, whereby heterocyclyl in each case stands especially for furyl, tetrahydrofuryl, thienyl, pyrrolyl, pyrrolinyl, pyrrolidinyl, pyrazolyl, pyrazolinyl, oxazolyl, oxazolinyl, isoxazolyl, isoxazolinyl, thiazolyl, isothiazolyl, oxadiazolyl, thiadiazolyl, dioxolanyl, dioxanyl, pyridinyl, piperidinyl, morpholinyl, pyrimidinyl or piperazinyl or for 2,4-dioxaspiro[5.5]undec-8-en-3-yl, 2,4-dioxaspiro[5.5]undecan-3-yl.
A¹, A², A³, A⁴ and A⁵ are the same or different and in each case stand most preferably for N (nitrogen) or the group C—R, wherein, however, the imidazoazine bicycle contains 2 or 3 N atoms and R in the group C—R in the individual cases can have each time the same or different meanings in accordance with the following definition.
R stands in each case most preferably for H (hydrogen), nitro, amino, cyano, fluorine, chlorine, bromine, iodine, or for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, methylamino, ethylamino, n- or i-propylamino, n-, i-, s- or t-butylamino, dimethylamino or diethylamino, in each case optionally substituted by cyano, fluorine, chlorine, bromine, methoxy, ethoxy, n- or i-propoxy, n-, i-, s- or t-butoxy, or optionally two neighbouring R groups together stand for propane-1,3-diyl, butane-1,3-diyl, butane-1,4-diyl, pentane-1,3-diyl, pentane-1,4-diyl or pentane-1,5-diyl, or optionally two neighbouring R groups, together with the azine group to which they are connected, form a benzene ring.
R¹ stands most preferably for CF₃.
X stands most preferably for H (hydrogen), hydroxycarbonyl (COOH), for aminocarbonyl optionally substituted by benzyloxycarbonyl, N,O-dimethylhydroxylaminocarbonyl, N,O-diethylhydroxylaminocarbonyl, N-methyl-O-ethylhydroxylaminocarbonyl or N-ethyl-O-methylhydroxylaminocarbonyl, for nitro, formyl, hydroxyiminomethyl, aminoiminomethyl, amino, cyano, fluorine, chlorine, bromine, iodine, for methyl, ethyl, n- or i-propyl, n-, i-, s- or t-butyl, n-pentyl, in each case optionally substituted by cyano, hydroxy, fluorine, chlorine, methoxy, ethoxy, n- or i-propoxy, methylcarbonyloxy, ethylcarbonyloxy, dimethylaminocarbonyloxy, methylamino, ethylamino, dimethylamino, diethylamino, dipropylamino, benzylamino or dibenzylamino, for methyl substituted by the group —NR′R″ (where R′R″ together with the nitrogen stands for pyrrolidine, piperidine, 4-trifluoromethylpiperidine, 3-trifluoromethylpiperidine, fluoromethylpiperidine, morpholine, dimethylmorpholine, piperazine or N-methylpiperazine), acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, methoximinomethyl, ethoximinomethyl, cyclopropylmethoxyiminomethyl, benzyloxyiminomethyl, chlorbenzyloxyiminomethyl, methylcarbonyloxyiminomethyl, allyloxyiminomethyl, phenylsulphonylaminoiminomethyl, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s- or t-butylamino, methylaminocarbonyl, ethylaminocarbonyl, n- or i-propylaminocarbonyl, dimethylamino, diethylamino, dimethylaminocarbonyl, acetylaminocarbonyl, propionylaminocarbonyl, n- or i-butyroylaminocarbonyl, N-methyl-acetylaminocarbonyl, N-methylpropionylaminocarbonyl, methoxycarbonylaminocarbonyl, ethoxycarbonylaminocarbonyl, n- or i-propoxycarbonylaminocarbonyl, N-methyl-methoxycarbonylaminocarbonyl, N-methylethoxycarbonylaminocarbonyl, in each case optionally substituted by cyano, hydroxy, fluorine, chlorine, methoxy, ethoxy, n- or i-propoxy, methylcarbonyloxy, ethylcarbonyloxy, dimethylaminocarbonyloxy, methylamino, ethylamino, dimethylamino, diethylamino, dipropylamino, benzylamino or dibenzylamino, for methylaminocarbonylaminocarbonyl, ethylaminocarbonylaminocarbonyl, n- or i-propylaminocarbonylaminocarbonyl, N-methylmethylaminocarbonylaminocarbonyl, N-methylethylaminocarbonylaminocarbonyl, N,O-dimethylhydroxylaminocarbonyl, N,O-diethylhydroxylaminocarbonyl, N-methyl-O-ethylhydroxylaminocarbonyl, N-ethyl-O-methylhydroxylaminocarbonyl), in each case optionally substituted by cyano, for ethenyl, propenyl, butenyl, pentenyl, propenyloxy, butenyloxy, pentenyloxy, propenylamino, butenylamino, pentenylamino, allyloximinomethyl, ethynyl, propynyl, butynyl, pentynyl, hexynyl, heptynyl, propynyloxy, butynyloxy, pentynyloxy, propynylaminocarbonyl, butynylaminocarbonyl or pentynylaminocarbonyl in each case optionally substituted by cyano, hydroxy, methoxy, ethoxy, n- or i-propoxy, pyridyl (which is optionally substituted by halogen), thienyl, thiazolyl (which itself is optionally substituted by methyl or ethyl), trialkylsilyl, phenyl (which itself is optionally substituted by methyl, ethyl, methoxy, ethoxy, fluorine, chlorine, bromine, iodine, or trifluoromethyl), phenoxy, methoxycarbonyl, ethoxycarbonyl, fluorine, chlorine or bromine, for cyclopropyl, cyclopentyl, cyclohexyl, cyclopropyloxy, cyclopentyloxy, cyclohexyloxy, cyclopropylamino, cyclopentylamino, cyclohexylamino, cyclopropylmethyl, cyclopentylmethyl, cyclohexyl methyl, cyclopropylmethoxy, cyclopentylmethoxy, cyclohexylmethoxy, cyclopropylmethylamino, cyclopentylmethylamino or cyclohexylmethylamino, in each case optionally substituted by cyano, fluorine, chlorine, bromine, iodine, methyl, ethyl, n- or i-propyl or trifluoromethyl, for dioxolan-2-yl, 1,3-dioxan-2-yl, oxazolyl, 1,2,4-oxadiazolyl, 1,2,4-thiadiazolyl, phenyl, phenoxy, phenylthio, phenylamino, benzyl, phenylethyl, phenylethynyl, phenylmethoxy, phenylethoxy, phenylmethylthio, phenylmethylamino or phenylethylamino, in each case optionally substituted by nitro, amino, hydroxy, cyano, carbamoyl, thio-carbamoyl, fluorine, chlorine, bromine, iodine, methyl, ethyl, n- or i-propyl, n-, i-, s- or t-butyl, trifluoromethyl, trichloromethyl, fluorodichloromethyl, chlorodifluoromethyl, methoxy, ethoxy, n- or i-propoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, methylthiomethyl, difluoromethylthio, trifluoromethylthio, chlorodifluoromethylthio, methylsulphinyl, ethylsulphinyl, trifluoromethylsulphinyl, methylsulphonyl, ethylsulphonyl, trifluoromethylsulphonyl, methylamino, ethylamino, n- or i-propylamino, dimethylamino, dimethylaminocarbonyl, dimethylaminosulphonyl, phenoxy, thienylsulphonylmethyl, piperidinomethyl, benzyl or phenyl, or for 2,4-dioxaspiro[5.5]undec-8-en-3-yl, 2,4-dioxaspiro[5.5]undecan-3-yl or phenylethylamino.

Preferred are compounds of structure (I) in which the group R¹ is in the ortho or para position relative to the nitrogen of the pyridine ring, more preferably in the para position.

A most particularly preferred group are compounds of the structure (IA)

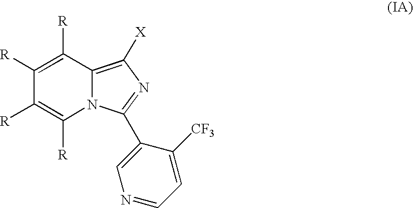

whereby in the structure (IA) R and X have the aforementioned meanings and preferences or
R which may be the same or different, and in each case stands most preferably for H (hydrogen), nitro, amino, cyano, fluorine, chlorine, bromine, iodine, or for methyl, ethyl, n- or i-propyl, methoxy, ethoxy, n- or i-propoxy, methylthio, ethylthio, n- or i-propylthio, methylsulphinyl, ethylsulphinyl, methylsulphonyl, ethylsulphonyl, methylamino, ethylamino, n- or i-propylamino, dimethylamino or diethylamino, in each case optionally substituted by cyano, fluorine, chlorine, bromine, methoxy, ethoxy, n- or i-propoxy, or in each case two neighbouring R groups together optionally stand for propane-1,3-diyl, butane-1,3-diyl, butane-1,4-diyl, pentane-1,3-diyl, pentane-1,4-diyl or pentane-1,5-diyl, or in each case optionally two neighbouring R groups, together with the neighbouring azine group to which they are connected, form a benzene ring, where in all cases a maximum two R groups are different to H (hydrogen), and
X stands most preferably for H (hydrogen), nitro, formyl, hydroximinomethyl, amino, cyano, fluorine, chlorine, bromine, iodine, for methyl, ethyl, n- or i-propyl, acetyl, propionyl, n- or i-butyroyl, methoxy, ethoxy, n- or i-propoxy, methoxycarbonyl, ethoxycarbonyl, n- or i-propoxycarbonyl, methoximinomethyl, ethoximinomethyl, methylthio, ethylthio, n- or i-propylthio, methylsulphinyl, ethylsulphinyl, methylsulphonyl, ethylsulphonyl, methylamino, ethylamino, n- or i-propylamino, methylaminocarbonyl, ethylaminocarbonyl, n- or i-propylaminocarbonyl, dimethylamino, diethylamino, dimethylaminocarbonyl, acetylamino, propionylamino, n- or i-butyroylamino, N-methylacetylamino, N-methylpropionylamino, methoxycarbonylamino, ethoxycarbonylamino, n- or i-propoxycarbonylamino, N-methylmethoxycarbonylamino, N-methylethoxycarbonylamino, methylaminocarbonylamino, ethylaminocarbonylamino, n- or i-propylamino-carbonylamino, N-methylmethylaminocarbonylamino, N-methyl-ethylaminocarbonylamino, in each case optionally cyano, fluorine, chlorine, methoxy, ethoxy, n- or i-propoxy, for ethenyl, propenyl, butenyl, propenyloxy, butenyloxy, propenylamino, butenylamino, ethynyl, propynyl, butynyl, propynyloxy, butynyloxy, propynylamino or butynylamino, in each case optionally substituted by cyano, fluorine, chlorine or bromine, for cyclopropyl, cyclopentyl, cyclohexyl, cyclopropyloxy, cyclopentyloxy, cyclohexyloxy, cyclopropylamino, cyclopentylamino, cyclohexylamino, cyclopropylmethyl, cyclopentylmethyl, cyclohexylmethyl, cyclopropylmethoxy, cyclopentylmethoxy, cyclohexylmethoxy, cyclopropylmethylamino, cyclopentylmethylamino or cyclohexylmethylamino, in each case optionally substituted by cyano, fluorine, chlorine, bromine, methyl, ethyl, n- or i-propyl or trifluoromethyl, for phenyl, phenoxy, phenylthio, phenylamino, benzyl, phenylethyl, phenylethynyl, phenylmethoxy, phenylethoxy, phenylmethylthio, phenylmethylamino, phenylethylamino or phenylethylamino, in each case optionally substituted by nitro, amino, hydroxy, cyano, carbamoyl, thiocarbamoyl, fluorine, chlorine, bromine, iodine, methyl, ethyl, n- or i-propyl, n-, i-, s- or t-butyl, trifluoromethyl, trichloromethyl, fluorodichloromethyl, chlorodifluoromethyl, methoxy, ethoxy, n- or i-propoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, difluoromethylthio, trifluoromethylthio, chlorodifluoromethylthio, methylsulphinyl, ethylsulphinyl, trifluoromethylsulphinyl, methylsulphonyl, ethylsulphonyl, trifluoromethylsulphonyl, methylamino, ethylamino, n- or i-propylamino, dimethylamino, dimethylaminocarbonyl, dimethylaminosulphonyl or phenyl.

A further most preferred group are the compounds of structure (IB),

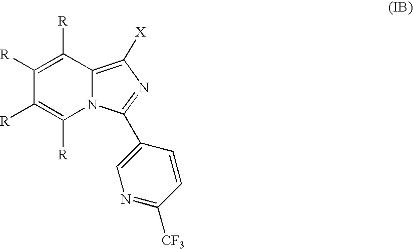

whereby in the structure (IB) R and X have the aforementioned meanings and preferences or
R which may be the same or different, stands most preferably for H (hydrogen), nitro, amino, cyano, fluorine, chlorine, bromine, iodine or for methyl, ethyl, n- or i-propyl, methoxy, ethoxy, n- or i-propoxy, methylthio, ethylthio, n- or i-propylthio, methylsulphinyl, ethylsulphinyl, methylsulphonyl, ethylsulphonyl, methylamino, ethylamino, n- or i-propylamino, dimethylamino or diethylamino in each case optionally substituted by cyano, fluorine, chlorine, bromine, methoxy, ethoxy, n- or i-propoxy, or in each case two neighbouring R groups optionally stand for propane-1,3-diyl, butane-1,3-diyl, butane-1,4-diyl, pentane-1,3-diyl, pentane-1,4-diyl or pentane-1,5-diyl, or optionally in each case two neighbouring R groups, together with the azine group to which they are connected, form a benzene ring, whereby in all cases a maximum of two R groups are different from H (hydrogen), and
X stands most preferably for H (hydrogen), nitro, formyl, hydroximinomethyl, amino, cyano, fluorine, chlorine, bromine, iodine, for methyl, ethyl, n- or i-propyl, acetyl, propionyl, n- or i-butyroyl, methoxy, ethoxy, n- or i-propoxy, methoxycarbonyl, ethoxycarbonyl, n- or i-propoxycarbonyl, methoximinomethyl, ethoximinomethyl, methylthio, ethylthio, n- or i-propylthio, methylsulphinyl, ethylsulphinyl, methylsulphonyl, ethylsulphonyl, methylamino, ethylamino, n- or i-propylamino, methylaminocarbonyl, ethylaminocarbonyl, n- or i-propylaminocarbonyl, dimethylamino, diethylamino, dimethylaminocarbonyl, acetylamino, propionylamino, n- or i-butyroylamino, N-methylacetylamino, N-methylpropionylamino, methoxycarbonylamino, ethoxycarbonylamino, n- or i-propoxycarbonylamino, N-methylmethoxycarbonylamino, N-methylethoxycarbonylamino, methylaminocarbonylamino, methylaminocarbonylamino, n- or i-propylaminocaibonylamino, N-methylmethylaminocarbonylamino, N-methylethylaminocarbonylamino, in each case optionally substituted by cyano, fluorine, chlorine, methoxy, ethoxy, n- or i-propoxy, for ethenyl, propenyl, butenyl, propenyloxy, butenyloxy, propenylamino, butenylamino, ethynyl, propynyl, butynyl, propynyloxy, butynyloxy, propynylamino or butynylamino, in each case optionally substituted by cyano, fluorine, chlorine or bromine, for cyclopropyl, cyclopentyl, cyclohexyl, cyclopropyloxy, cyclopentyloxy, cyclohexyloxy, cyclopropylamino, cyclopentylamino, cyclohexylamino, cyclopropylmethyl, cyclopentylmethyl, cyclohexylmethyl, cyclopropylmethoxy, cyclopentylmethoxy, cyclohexylmethoxy, cyclopropylmethylamino, cyclopentylmethylamino or cyclohexylmethylamino, in each case optionally substituted by cyano, fluorine, chlorine, bromine, methyl, ethyl, n- or i-propyl or trifluoromethyl, for phenyl, phenoxy, phenylthio, phenylamino, benzyl, phenylethyl, phenylethynyl, phenylmethoxy, phenylethoxy, phenylmethylthio, phenylmethylamino, phenylethylamino or phenylethylamino in each case optionally substituted by nitro, amino, hydroxy, cyano, carbamoyl, thiocarbamoyl, fluorine, chlorine, bromine, iodine, methyl, ethyl, n- or i-propyl, n-, i-, s- or t-butyl, trifluoromethyl, trichloromethyl, fluorodichloromethyl, chlorodifluoromethyl, methoxy, ethoxy, n- or i-propoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, difluoromethylthio, trifluoromethylthio, chlorodifluoromethylthio, methylsulphinyl, ethylsulphinyl, trifluoromethylsulphinyl, methylsulphonyl, ethylsulphonyl, trifluoromethylsulphonyl, methylamino, ethylamino, n- or i-propylamino, dimethylamino, dimethylaminocarbonyl, dimethylaminosulphonyl or phenyl.

Particularly emphasised are also compounds with the following preferred residue combinations:
A¹, A², A³, A⁴ and A⁵ are the same or different and in each case stand preferably for N (nitrogen) or the group C—R, whereby, however, the imidazoazine bicycle contains 2 to 5 N atoms and in no case are more than two N atoms next to each other, and R in the group C—R in the individual cases can have in each case the same or different meanings in accordance with the following definitions.
R stands in each case preferably for H (hydrogen), nitro, amino, cyano, halogen, or for alkyl, alkoxy, alkylthio, alkylsulphinyl, alkylsulphonyl, alkylamino or dialkylamino with in each case 1 to 6 carbon atoms in the alkyl groups and in each case optionally substituted by cyano, halogen or C₁-C₄-alkoxy, or optionally two neighbouring R groups together stand for alkanediyl with 3 to 5 carbon atoms, or optionally two neighbouring R groups, together with the azine group to which they are connected, form a benzene ring.
R¹ stands preferably for CF₃, CHF₂ or CF₂Cl.
X stands preferably for H (hydrogen), nitro, formyl, hydroximinomethyl (—CH═N—OH), aminoiminomethyl (—CH═N—NH2), amino, cyano, NCO (isocyanato), NCS (isothiocyanato), halogen, for alkyl with 1 to 6 carbon atoms, optionally substituted by cyano, hydroxy, halogen, C₁-C₄-alkoxy, C₁-C₄-alkylamino or di(C₁-C₄-alkyl)-amino, for alkylcarbonyl, alkoxy, alkoxycarbonyl, alkoximinomethyl (—CH═N—O-alkyl), alkylaminoiminomethyl (—CH═N—NH-alkyl), dialkylaminoiminomethyl, alkylthio, alkylsulphinyl, alkylsulphonyl, alkylamino, alkylaminocarbonyl, dialkylamino, dialkylaminocarbonyl, alkylcarbonylamino, N-alkyl-alkylcarbonylamino, alkoxycarbonylamino, N-alkyl-alkoxycarbonylamino, alkylaminocarbonylamino or N-alkyl-N-alkylaminocarbonylamino with each case 1 to 6 carbon atoms in the alkyl groups, in each case optionally substituted cyano, hydroxy, halogen or C₁-C₄-alkoxy, for alkenyl, alkenyloxy, alkenylamino, alkenyloximinomethyl, alkynyl, alkynyloxy or alkynylamino with in each case 2 to 6 carbon atoms in the alkenyl or alkynyl groups, in each case optionally substituted by cyano, hydroxy, phenoxy or halogen, for cycloalkyl, cycloalkyloxy, cycloalkylalkoximinomethyl, cycloalkylamino, cycloalkylalkyl, cycloalkylalkoxy or cycloalkylalkylamino with in each case 3 to 6 carbon atoms in the cycloalkyl groups and optionally 1 to 4 carbon atoms in the alkyl parts, in each case optionally substituted by cyano, halogen, C₁-C₄-alkyl or C₁-C₄-haloalkyl, for aryl, aryloxy, arylthio, arylamino, arylaminoiminomethyl, arylalkyl, arylethynyl, arylalkoxy, arylalkylthio, arylalkylamino, arylalkylaminoiminomethyl, arylalkoxyiminomethyl or arylsulphonylaminoiminomethyl with in each case 6 or 10 carbon atoms in the aryl group and optionally 1 to 4 carbon atoms in the alkyl part, in each case substituted by nitro, amino, hydroxy, cyano, carbamoyl, thiocarbamoyl, halogen, C₁-C₄-alkyl, C₁-C₄-halogenalkyl, C₁-C₄-alkoxy, C₁-C₄-halogenalkoxy, C₁-C₄-alkylthio, C₁-C₄-haloalkylthio, C₁-C₄-alkylsulphinyl, C₁-C₄-halogalkylsulphinyl, C₁-C₄-alkylsulphonyl, C₁-C₄-haloalkylsulphonyl, C₁-C₄-alkylamino, di(C₁-C₄-alkyl)-amino, di(C₁-C₄-alkyl)aminocarbonyl, di(C₁-C₄-alkyl)aminosulphonyl or phenyl, or for heterocyclyl, heterocyclyloxy, heterocyclylthio, heterocyclylamino, heterocyclylalkyl, heterocyclylalkynyl, heterocyclylalkoxy, heterocyclylalkylthio or heterocyclylalkylamino with in each case up to 8 carbon atoms and at least one heteroatom from the series N (nitrogen), O (oxygen), S (sulphur) and optionally also a group CO, CS, SO or SO₂ as component of the heterocycle as well as up to 4 carbon atoms in the alkyl part or alkynyl part, in each case optionally substituted by nitro, amino, hydroxy, cyano, carbamoyl, thio-carbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulphinyl, C₁-C₄-halogenalkylsulphinyl, C₁-C₄-alkylsulphonyl, C₁-C₄-haloalkylsulphonyl, C₁-C₄-alkylamino, di(C₁-C₄-alkyl)amino, di(C₁-C₄-alkyl)amino-carbonyl, di(C₁-C₄-alkyl)amino-sulphonyl or phenyl.
A¹, A², A³, A⁴ and A⁵ are the same or different and in each case stand more preferably for N (nitrogen) or the group C—R, whereby, however, the imidazoazine bicycle contains 2 to 4 nitrogen atoms and whereby R in the groups C—R can have in each case the same or different meanings in accordance with the following definition.
R stands in each case more preferably for H (hydrogen), nitro, amino, cyano, fluorine, chlorine, bromine, iodine, or for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, methylamino, ethylamino, n- or i-propylamino, n-, i-, s- or t-butylamino, dimethylamino or diethylamino, in each case optionally substituted by cyano, fluorine, chlorine, bromine, methoxy, ethoxy, n- or i-propoxy, n-, i-, s- or t-butoxy, or optionally two neighbouring R groups together stand for propane-1,3-diyl, butane-1,3-diyl, butane-1,4-diyl, pentane-1,3-diyl, pentane-1,4-diyl or pentane-1,5-diyl, or optionally two neighbouring R groups, together with the azine group to which they are connected, form a benzene ring.
R¹ stands preferably for CF₃.
X stands more preferably for H (hydrogen), nitro, formyl, hydroximinomethyl (—CH═N—OH), aminoiminomethyl (—CH═N—NH2), amino, cyano, NCO (isocyanato), NCS (isothiocyanato), fluorine, chlorine, bromine, iodine, for methyl, ethyl, n- or i-propyl, n-, i-, s- or t-butyl, n-, i-, s-, t- or neo-pentyl, in each case optionally substituted by cyano, hydroxy, fluorine, chlorine, bromine, methoxy, ethoxy, n- or i-propoxy, n-, i-, s- or t-butoxy, methylamino, ethylamino, n- or i-propylamino, n-, i-, s- or t-butylamino, dimethylamino or diethylamino, for acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, methoximinomethyl, ethoximinomethyl, methylaminoiminomethyl, ethylaminoiminomethyl, n- or i-propylaminoiminomethyl, dimethylaminoiminomethyl, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s- or t-butylamino, methylaminocarbonyl, ethylaminocarbonyl, n- or i-propylaminocarbonyl, dimethylamino, diethylamino, di-n-propylamino, di-i-propylamino, dimethylaminocarbonyl, diethylaminocarbonyl, acetylamino, propionylamino, n- or i-butyroylamino, N-methylacetylamino, N-methylpropionylamino, methoxycarbonylamino, ethoxycarbonylamino, n- or i-propoxycarbonylamino, N-methylmethoxycarbonylamino, N-methylethoxycarbonylamino, methylaminocarbonylamino, ethylaminocarbonylamino, n- or i-propylaminocarbonylamino, N-methylmethylaminocarbonylamino, N-methylethylaminocarbonylamino, in each case optionally substituted by cyano, hydroxy, fluorine, chlorine, bromine, methoxy, ethoxy, n- or i-propoxy, n-, i-, s- or t-butoxy, for ethenyl, propenyl, butenyl, pentenyl, hexenyl, propenyloxy, butenyloxy, pentenyloxy, propenylamino, butenylamino, pentenylamino, allyloximinomethyl, ethynyl, propynyl, butynyl, pentynyl, hexynyl, propynyloxy, butynyloxy, pentynyloxy, propynylamino, butynylamino or pentynylamino, in each case optionally substituted by cyano, hydroxy, phenoxy, fluorine, chlorine or bromine, for cyclopropyl, cyclobutyl, cyclopentyl, cyclohexyl, cyclopropyloxy, cyclobutyloxy, cyclopentyloxy, cyclohexyloxy, cyclopropylamino, cyclobutylamino, cyclopentylamino, cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, ccyclopropylmethoximinomethyl, cyclopropylmethylamino, cyclobutylmethylamino, cyclopentylmethylamino or cyclohexylmethylamino, in each case optionally substituted by cyano, fluorine, chlorine, bromine, iodine, methyl, ethyl, n- or i-propyl, n-, i-, s- or t-butyl, fluoromethyl, chloromethyl, difluoromethyl, dichloromethyl, trifluoromethyl or trichloromethyl, for phenyl, naphthyl, phenoxy, naphthyloxy, phenylthio, naphthylthio, phenylamino, naphthylamino, phenylaminoiminomethyl, benzyl, phenylethyl, phenylpropyl, phenylethynyl, phenylmethoxy, phenylethoxy, phenylpropoxy, phenylmethylthio, phenylmethylamino, phenylethylamino, phenylmethylaminoiminomethyl, phenylmethoxyiminomethyl or phenylsulphonylaminoiminomethyl, in each case optionally substituted by nitro, amino, hydroxy, cyano, carbamoyl, thiocarbamoyl, fluorine, chlorine, bromine, iodine, methyl, ethyl, n- or i-propyl, n-, i-, s- or t-butyl, fluoromethyl, chloromethyl, difluoromethyl, dichloromethyl, trifluoromethyl, trichloromethyl, fluorodichloromethyl, chlorodifluoromethyl, methoxy, ethoxy, n- or i-propoxy, n-, i-, s- or t-butoxy, difluoromethoxy, trifluoroomethoxy, chlorodifluoromethoxy, fluoroethoxy, chloroethoxy, difluoroethoxy, dichloroethoxy, trifluoroethoxy, methylthio, ethylthio, n- or i-propylthio, n-, i-, s- or t-butylthio, difluoromethylthio, trifluoromethylthio, chlorodifluoromethylthio, methylsulphinyl, ethylsulphinyl, n- or i-propylsulphinyl, trifluoromethylsulphinyl, methylsulphonyl, ethylsulphonyl, trifluoromethylsulphonyl, methylamino, ethylamino, n- or i-propylamino, n-, i-, s- or t-butylamino, dimethylamino, diethylamino, dimethylaminocarbonyl, diethylaminocarbonyl, dimethylaminosulphonyl, diethylaminosulphonyl or phenyl, or for heterocyclyl, heterocyclyloxy, heterocyclylthio, heterocyclylamino, heterocyclylmethyl, heterocyclylethynyl, heterocyclylmethoxy, heterocyclylmethylthio or heterocyclylmethylamino, in each case optionally substituted by nitro, amino, hydroxy, cyano, carbamoyl, thiocarbamoyl, fluorine, chlorine, bromine, iodine, methyl, ethyl, n- or i-propyl, n-, i-, s- or t-butyl, fluoromethyl, chloromethyl, difluoromethyl, dichloromethyl, trifluoromethyl, trichloromethyl, fluorodichloromethyl, chlorodifluoromethyl, methoxy, ethoxy, n- or i-propoxy, n-, i-, s- or t-butoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, n-, i-, s- or t-butylthio, difluoromethylthio, trifluoromethylthio, chlorodifluoromethylthio, methylsulphinyl, ethylsulphinyl, n- or i-propylsulphinyl, trifluoromethylsulphinyl, methylsulphonyl, ethylsulphonyl, trifluoromethylsulphonyl, methylamino, methylamino, n- or i-propylamino, n-, i-, s- or t-butylamino, dimethylamino, diethylamino, dimethylaminocarbonyl, diethylaminocarbonyl, dimethylaminosulphonyl, diethylaminosulphonyl or phenyl, whereby in each case the heterocycle stands preferably for furyl, tetrahydrofuryl, thienyl, pyrrolyl, pyrrolinyl, pyrrolidinyl, pyrazolyl, pyrazolinyl, oxazolyl, oxazolinyl, isoxazolyl, isoxazolinyl, thiazolyl, isothiazolyl, oxadiazolyl, thiadiazolyl, pyridinyl, pyrrolidinyl, morpholinyl, piperazinyl or pyrimidinyl.
A¹, A², A³, A⁴ and A⁵ are the same or different and in each case stand most preferably for N (nitrogen) or the group C—R, whereby, however, the imidazoazine bicycle contains 2 or 3 nitrogen atoms and whereby R in the groups C—R in the individual case can have in each case the same or different meanings in accordance with the following definition.
R stands in each case most preferably for H (hydrogen), nitro, amino, cyano, fluorine, chlorine, bromine, iodine, or for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, methylamino, ethylamino, n- or i-propylamino, n-, i-, s- or t-butylamino, dimethylamino or diethylamino in each case optionally substituted by cyano, fluorine, chlorine, bromine, methoxy, ethoxy, n- or i-propoxy, n-, i-, s- or t-butoxy, or optionally two neighbouring R groups stand for propane-1,3-diyl, butane-1,3-diyl, butane-1,4-diyl, pentane-1,3-diyl, pentane-1,4-diyl or pentane-1,5-diyl, or optionally two neighbouring R groups together with the azine group to which they are connected form a benzene ring.
R¹ stands most preferably for CF₃.
X stands most preferably for H (hydrogen), nitro, formyl, hydroximinomethyl, aminoiminomethyl, amino, cyano, NCO (isocyanato), NCS (isothiocyanato), fluorine, chlorine, bromine, iodine, for methyl, ethyl, n- or i-propyl, n-, i-, s- or t-butyl, acetyl, propionyl, n- or i-butyroyl, methoxy, methoxy, n- or i-propoxy, n-, i-, s- or t-butoxy, methoxycarbonyl, ethoxycarbonyl, n- or i-propoxycarbonyl, methoximinomethyl, ethoximinomethyl, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s- or t-butylamino, methylaminocarbonyl, ethylaminocarbonyl, n- or i-propylaminocarbonyl, dimethylamino, diethylamino, dimethylaminocarbonyl, acetylamino, propionylamino, n- or i-butyroylamino, N-methyl-acetylamino, N-methyl-propionylamino, methoxycarbonylamino, ethoxycarbonylamino, n- or i-propoxycarbonylamino, N-methylmethoxycarbonylamino, N-methylethoxycarbonylamino, methylaminocarbonylamino, ethylaminocarbonylamino, n- or i-propylamino-carbonylamino, N-methyl-methylaminocarbonylamino, N-methylethylaminocarbonylamino in each case optionally substituted by cyano, hydroxy, fluorine, chlorine, methoxy, ethoxy, n- or i-propoxy, for ethenyl, propenyl, butenyl, pentenyl, propenyloxy, butenyloxy, pentenyloxy, propenylamino, butenylamino, pentenylamino, allyloximinomethyl, ethynyl, propynyl, butynyl, pentynyl, propynyloxy, butynyloxy, pentynyloxy, propynylamino, butynylamino or pentynylamino, in each case optionally substituted by cyano, fluorine, chlorine or bromine, for cyclopropyl, cyclopentyl, cyclohexyl, cyclopropyloxy, cyclopentyloxy, cyclohexyloxy, cyclopropylamino, cyclopentylamino, cyclohexylamino, cyclopropylmethyl, cyclopentylmethyl, cyclohexylmethyl, cyclopropylmethoxy, cyclopentylmethoxy, cyclohexylmethoxy, cyclopropylmethylamino, cyclopentylmethylamino or cyclohexylmethylamino, in each case optionally substituted by cyano, fluorine, chlorine, bromine, iodine, methyl, ethyl, n- or i-propyl or trifluoromethyl, for phenyl, phenoxy, phenylthio, phenylamino, benzyl, phenylethyl, phenylethynyl, phenylmethoxy, phenylethoxy, phenylmethylthio, phenylmethylamino, phenylethylamino, phenylethylamino or pyridinylethynyl, in each case optionally substituted by nitro, amino, hydroxy, cyano, carbamoyl, thiocarbamoyl, fluorine, chlorine, bromine, iodine, methyl, ethyl, n- or i-propyl, n-, i-, s- or t-butyl, trifluoromethyl, trichloromethyl, fluorodichloromethyl, chlorodifluoromethyl, methoxy, ethoxy, n- or i-propoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, difluoromethylthio, trifluoromethylthio, chlorodifluoromethylthio, methylsulphinyl, ethylsulphinyl, trifluoromethylsulphinyl, methylsulphonyl, ethylsulphonyl, trifluoromethylsulphonyl, methylamino, ethylamino, n- or i-propylamino, dimethylamino, dimethylaminocarbonyl, dimethylaminosulphonyl or phenyl.

The new azinylimidazoazines of the general structure (I) are obtained when a N-azinylalkylazino carboxamides of the general structure (II),

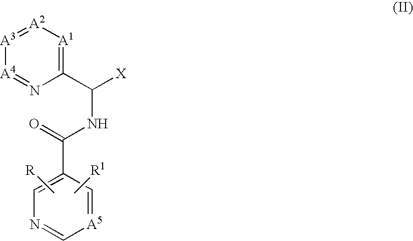

in which
A¹, A², A³, A⁴, A⁵, R, R¹ and X have the aforementioned meaning,
   is reacted with a condensation agent optionally in the presence of a diluent,
   and optionally the compounds of structure (I) are converted into another compound of structure (I) according to conventional methods within the context of the above substituent definition.

If, for example, N-(pyridine-2-yl-methyl)-4-trifluoromethyl-nicotinamide is used as starting material the course of the reaction in the process of the invention can be outlined by the following reaction scheme:

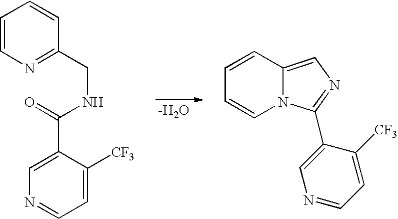

The N-azinylalkylazine carboxamides used as starting materials for the preparation of compounds of general structure (I) by the method of the invention are defined in general by structure (II). In the general structure (II) A¹, A², B, X, Y¹, Y² and Y³ have preferably and especially, respectively, those meanings which have already been described above in connection with the description of compounds of structure (I) of the invention as preferable or more preferable, respectively, for A¹, A², B, X, Y¹, Y² and Y³.

With the exception of the compounds N-(2-pyridinylmethyl)-4-trifluoromethylpyridine-3-carboxamide (cf. JP-07010841—cited in Chem. Abstracts 123:32961), 4-trifluoromethyl-N-[(5-trifluoromethyl-2-pyridinyl)methyl]pyridine-3-carboxamide, 4-trifluoromethyl-N-[(2,6-dichlor-4-pyridinyl)methyl]pyridine-3-carboxamide, 4-trifluoromethyl-N-[(6-chloro-2-pyridinyl)methyl]pyridine-3-carboxamide, 4-trifluoromethyl-N-[(2,3,5,6-tetrachloro-4-pyridinyl)methyl]pyridine-3-carboxamide, 4-trifluoromethyl-N-[(2-pyridinyl)methyl]pyridine-3-carboxamide, 4-trifluoromethyl-N-[(3-trifluoromethyl-2-pyridinyl)methyl]pyridine-3-carboxamide, 4-trifluoromethyl-N-[(5,6-dichloro-3-pyridinyl)methyl]pyridine-3-carboxamide, 4-trifluoromethyl-N-[(6-chloro-3-pyridinyl)methyl]pyridine-3-carboxamide, 4-trifluoromethyl-N-[(2-chloro-3-pyridinyl)methyl]pyridine-3-carboxamide, 4-trifluoromethyl-N-[(3-quinolinyl)methyl]pyridine-3-carboxamide, 4-trifluoromethyl-N-[(6-trifluoromethyl-2-pyridinyl)methyl]-pyridine-3-carboxamide, N-[(2-pyrazinyl)methyl]-4-trifluoromethylpyridine-3-carboxamide and N-[(3-chloro-5-trifluoromethyl-2-pyridinyl)methyl]-4-trifluoromethylpyridine-3-carboxamide (cf JP-07025853-cited in Chem. Abstracts 123:55702), 2-bromo-6-trifluoromethyl-N-[(3-chloro-5-trifluoromethyl-2-pyridinyl)methyl]pyridine-3-carboxamide, N-[(3-chloro-5-trifluoromethyl-2-pyridinyl)methyl]-2-methyl-6-trifluoromethylpyridine-3-carboxamide, N-[(3-chloro-5-trifluoromethyl-2-pyridinyl)methyl]-2-methoxy-6-trifluoromethylpyridine-3-carboxamide, N-[(3-chloro-5-trifluoromethyl-2-pyridinyl)methyl]-2-methoxymethyl-6-trifluoromethylpyridine-3-carboxamide, N-[3-chloro-5-trifluoromethyl-2-pyridinyl)methyl]-6-trifluoromethylpyridine-3-carboxamide (cf WO-2002/022583) and N-[[3-chloro-5-(trifluoromethyl)pyridin-2-yl](piperidin-1-yl)methyl]-4-(trifluoromethyl)nicotinamide (cf. WO-2001/011966) the starting materials of the general structure (II) are not known in the literature.

With the exception of the compounds N-(2-pyridinylmethyl)-4-trifluoromethyl-pyridine-3-carboxamide (cf. JP-07010841—cited in Chem. Abstracts 123:32961), 4-trifluoromethyl-N-[(5-trifluoromethyl-2-pyridinyl)methyl]pyridine-3-carboxamide, 4-trifluoromethyl-N-[(2,6-dichloro-4-pyridinyl)methyl]pyridine-3-carboxamide, 4-trifluoromethyl-N-[(6-chloro-2-pyridinyl)methyl]pyridine-3-carboxamide, 4-trifluoromethyl-N-[(2,3,5,6-tetrachloro-4-pyridinyl)methyl]pyridine-3-carboxamide, 4-trifluoromethyl-N-[(2-pyridinyl)methyl]pyridine-3-carboxamide, 4-trifluoromethylN-[(3-trifluoromethyl-2-pyridinyl)methyl]pyridine-3-carboxamide, 4-trifluoromethyl-N-[(5,6-dichloro-3-pyridinyl)methyl]pyridine-3-carboxamide, 4-trifluoromethyl-N-[(6-chloro-3-pyridinyl)methyl]pyridine-3-carboxamide, 4-trifluoromethyl-N-[(2-chloro-3-pyridinyl)methyl]pyridine-3-carboxamide, 4-trifluoromethyl-N-[(3-quinolinyl)methyl]pyridine-3-carboxamide, 4-trifluoromethyl-N-[(6-trifluoromethyl-2-pyridinyl)methyl]pyridine-3-carboxamide, N-[(2-pyrazinyl)methyl]-4-trifluoromethylpyridine-3-carboxamide and N-[(3-chloro-5-trifluoromethyl-2-pyridinyl)methyl]-4-trifluoromethylpyridine-3-carboxamide (cf. JP-07025853—cited in Chem. Abstracts 123:55702), 2-bromo-6-trifluoromethyl-N-[(3-chloro-5-trifluoromethyl-2-pyridinyl)methyl]pyridine-3-carboxamide, N-[(3-chloro-5-trifluoromethyl-2-pyridinyl)methyl]-2-methyl-6-trifluoromethylpyridine-3-carboxamide, N-[(3-chloro-5-trifluoromethyl-2-pyridinyl)methyl]-2-methoxy-6-trifluoromethylpyridine-3-carboxamide, N-[(3-chloro-5-trifluoromethyl-2-pyridinyl)methyl]2-methoxymethyl-6-trifluoromethylpyridine-3-carboxamide, N-[3-chloro-5-trifluoromethyl-2-pyridinyl)methyl]-6-trifluoromethylpyridine-3-carboxamide (cf. WO-2002/022583) and N-[[3-chloro-5-(trifluoromethyl)pyridin-2-yl](piperidin-1-yl)methyl]-4-(trifluoromethyl)nicotinamide (cf. WO-2001/011966) the compounds of structure (II) are, as new compounds, subject matter of the present application.

The heterocycle containing the substituents A¹, A², A³ and A⁴ contains one nitrogen atom less than the corresponding imidazoazine bicycle in the final product.

A further subject matter is the use of the compounds of structure (II) as intermediates in the preparation of active agrochemical compounds, especially insecticides and fungicides.

According to the invention it was also found that the compounds of structure (II) themselves are, like the compounds of structure (I), equally suitable in a special way to control undesirable micro-organisms in and/or on plants and/or zoopests. The compounds are therefore particularly highly active insecticides.

Specially mentioned in this connection is the following group of compounds of structure (II-b), which according to the invention have been found to be especially advantageous active compounds

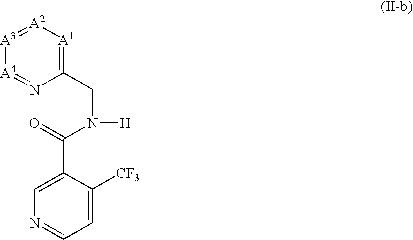

whereby in structure (II-b)
A¹, A², A³, and A⁴ are the same or different and in each case stand for N (nitrogen) or the group C—R, whereby, however, the heterocycle containing the substituents A¹, A², A³ and A⁴ in every case contains 2 or 3 N atoms and in no case are more than two N atoms next to each other, and whereby R in the groups C—R in the individual cases can have in each case the same or different meanings in accordance with the following definition.
R in each case stands for H (hydrogen), nitro, amino, cyano, halogen, or in each case for optionally substituted alkyl, alkoxy, alkylthio, alkylsulphinyl, alkylsulphonyl, alkylamino or dialkylamino, or optionally two neighbouring R groups together stand for alkanediyl, or, together with the azine group to which they are connected, form a benzene ring,
   except compounds in which the heterocycle containing the substituents A¹, A², A³ and A⁴ stands for unsubstituted pyrazinyl.

The substituents A¹, A², A³, and A⁴ and R in the structure (II-b) have especially the following preferred meanings:
A¹, A², A³ and A⁴ are the same or different and in each case stand preferably for N (nitrogen) or the group C—R, whereby, however, the heterocycle containing the substituents A¹, A², A³ and A⁴ in every case contains 2 or 3 N atoms whereby in no case are more than two N atoms next to each other, and whereby R in the groups C—R in the individual cases can have in each case the same or different meanings in accordance with the following definitions.
R stands in each case preferably for H (hydrogen), nitro, amino, cyano, halogen, or for alkyl, alkoxy, alkylthio, alkylsulphinyl, alkylsulphonyl, alkylamino or dialkylamino with in each case 1 to 6 carbon atoms in the alkyl groups, in each case optionally substituted by cyano, halogen or C₁-C₄-alkoxy or optionally two neighbouring R groups stand together for alkanediyl with 3 to 5 carbon atoms, or, together with the azine group with which they are connected, form a benzene ring.
A¹, A², A³ and A⁴ are the same or different and in each case stand more preferably for N (nitrogen) or the group C—R, whereby, however, the heterocycle containing the substituents A¹, A², A³ and A⁴ in every case contains 2 or 3 N atoms whereby in no case are more than two N atoms next to each other, and whereby R in the groups C—R in the individual cases can have in each case the same or different meanings in accordance with the following definition.
R stands in each case more preferably for H (hydrogen), nitro, amino, cyano, fluorine, chlorine, bromine, iodine, or for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, methylamino, ethylamino, n- or i-propylamino, n-, i-, s- or t-butylamino, dimethylamino or diethylamino, in each case optionally substituted by cyano, fluorine, chlorine, bromine, methoxy, ethoxy, n- or i-propoxy, n-, i-, s- or t-butoxy, or optionally two neighbouring R groups stand for propane-1,3-diyl, butane-1,3-diyl, butane-1,4-diyl, pentane-1,3-diyl, pentane-1,4-diyl or pentane-1,5-diyl or together with the azine group to which they are connected form a benzene ring.
A¹, A², A³ and A⁴ are the same or different and in each case stand most preferably for N (nitrogen) or the group C—R, whereby, however, the heterocycle containing the substituents A¹, A², A³ and A⁴ in every case contains 2 or 3 N atoms, and whereby R in the groups C—R in the individual case can have in each case the same or different meanings in accordance with the following definitions.
R stands in each case most preferably for H (hydrogen), nitro, amino, cyano, fluorine, chlorine, bromine, iodine, or for methyl, ethyl, n- or i-propyl, n-, i-, s- or t-butyl, methoxy, ethoxy, n- or i-propoxy, n-, i-, s- or t-butoxy, methylthio, ethylthio, n- or i-propylthio, n-, i-, s- or t-butylthio, methylsulphinyl, ethylsulphinyl, n- or i-propylsulphinyl, methylsulphonyl, ethylsulphonyl, methylamino, ethylamino, n- or i-propylamino, n-, i-, s- or t-butylamino, dimethylamino or diethylamino in each case optionally substituted by cyano, fluorine, chlorine, bromine, methoxy, ethoxy, n- or i-propoxy, n-, i-, s- or t-butoxy, or optionally two neighbouring R groups together stand for propane-1,3-diyl, butane-1,3-diyl, butane-1,4-diyl, pentane-1,3-diyl, pentane-1,4-diyl or pentane-1,5-diyl, or optionally two neighbouring groups, together with the azine group to which they are connected, form a benzene ring.

Particularly mentioned are especially such compounds of structure (II-b) in which the heterocycle containing the substituents A¹, A², A³ and A⁴ stands for one of the following groups,

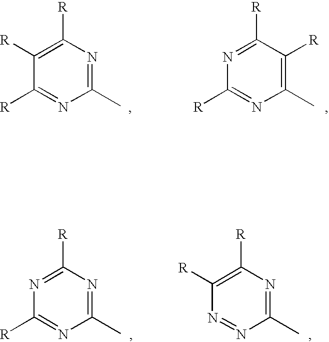

wherein the R residues are the same or different and have one of the aforementioned meanings.

Most particularly mentioned are such compounds in which the heterocycle containing the substituents A¹, A², A³ and A⁴ stands for one of the following groups

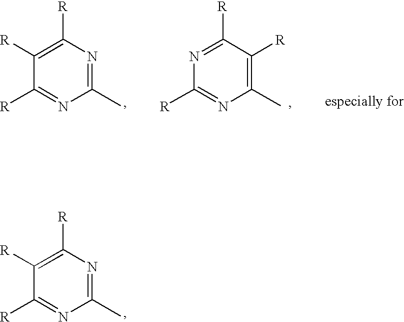

wherein the R residues are the same or different and have one of the aforementioned meanings.

The aforementioned general or preferred range residue definitions apply both to the final product of structure (I) as well correspondingly to the respective starting or intermediate products of structure (II) required for the preparation. These residue definitions can be arbitrarily combined between each other, as well as also between the aforementioned preferred ranges.

Preferred according to the invention are the compounds of structure (I) or (II-b) in which a combination of the meanings previously described as preferred is present.

More preferred according to the invention are the compounds of structure (I) or (II-b) in which a combination of the meanings previously described as more preferred is present.

Most preferred according to the invention are the compounds of structure (I) or (II-b) in which a combination of the meanings previously described as most preferred is present.

In the above and following residue definitions hydrocarbon residues such as alkyl—also in association with a heteroatom as in alkoxy—can be where possible in each case straight-chained or branched.

Depending upon the nature of the substituents the compounds of structure (I) or (II) or (II-b) can optionally also be present as stereoisomers, that is as geometric and/or optical isomers or isomer mixtures in different compositions. Both the pure stereoisomers and arbitrary mixtures of these isomers are subject matter of this invention, also when in general only compounds of structure (I) or (II) or (II-b) are discussed.

Depending upon the nature of the above defined substituents the compounds of structure (I) or (II) or (II-b) exhibit acidic or basic properties and can form salts. If the compounds of structure (I) carry hydroxy, carboxy or other groups imparting acidic properties these compounds can be converted into salts with bases. Suitable bases are, for example, hydroxides, carbonates, hydrogen carbonates of the alkali and alkaline earth metals, in particular those of sodium; potassium, magnesium and calcium, also ammonia, primary, secondary and tertiary amines with (C₁-C₄)-alkyl residues as well as mono-, di- and trialkanolamines of (C₁-C₄)-alkanols. If the compounds of structure (I) carry amino, alkylamino or other groups imparting basic properties these compounds can be converted into salts with acids. Suitable acids are, for example, mineral acids such as hydrochloric acid, sulphuric acid and phosphoric acid, organic acids such as acetic acid or oxalic acid, and acid salts such as NaHSO₄ and KHSO₄. The salts so obtained also exhibit fungicidal, insecticidal, acaricidal and miticidal properties.

The subject matter of the invention is also the salt-like derivatives formed from compounds of structure (I) or (II) or (II-b) by conversion with basic or acidic compounds as well as the N-oxides formed by normal oxidation methods.

The new azinylimidazoazines of general structure (I) or (II) or (II-b) exhibit interesting biological properties. They are characterised in particular by strong arthropodicidal (insecticidal and acaricidal) and nematocidal activity and can be used in agriculture, in forestry, storage and material protection as well in the hygiene sector.

The azinylalkylazine carboxamides of general structure (II) are obtained when an azino halocarbonyl derivatives of the general structure (III),

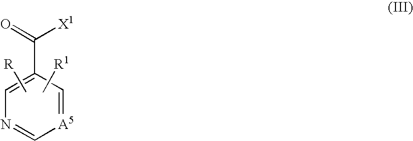

in which
A⁵, R and R¹ have the aforementioned meaning and
X¹ stands for halogen,
   is reacted with azinylalkylamines of the general structure (IV),

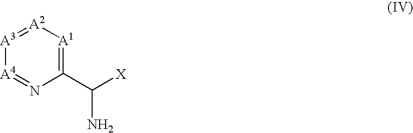

in which
A¹, A², A³, A⁴ and X have the aforementioned meaning,
   optionally in the presence of a diluent such as, for example, methylene chloride and optionally in the presence of a reaction auxiliary, such as, for example, triethylamine, at temperatures between 0° C. and 150° C.

The azine halocarbonyls of general structure (III) are known and/or can be prepared by known methods (cf. JP-03081263—cited in Chem. Abstracts 115:183112; JP-07010841—cited in Chem. Abstracts 123:32961; JP-07025853—cited in Chem. Abstracts 123:55702; WO-2000/015615; WO-2001/064674; WO-2003/044013). See also EP-A 185 256, EP-A 580 374, WO 00/35912, WO 01/09104, WO 01/70692, EP-A 185 256, EP-A 580 374, WO 00/35912, WO 01/09104, WO 01/70692.

The azinylalkylamines of general structure (IV) are also known and/or can be prepared by known methods (cf. J. Heterocycl. Chem. 17 (1980), 1061-1064; J. Med. Chem. 46 (2003), 461-473; J. Org. Chem. 40 (1975), 1210-1213; Synthesis 1996, 991-996; EP-361791; U.S. Pat. No. 4,555,573; U.S. Pat. No. 5,656,253; US-2003134836; WO-94/03427; WO-95/28400; WO-96/24609; WO-2000/017163; WO-2000/074682; WO-2000/075134; WO-2001/023387; WO-2001/038323; WO-2003/048133). Pyrimidine alkylamines can be prepared, for example, by hydrogenation of cyanopyrimidines with hydrogen in the presence of a palladium catalyst and in the presence of methanol as diluent or as reported in JP 2004/083495. The cyanopyrimidines, as well as cyanopyrazines, and routes to their preparation are known from, for example: JP 2000119258, 2000, Synth. Commun. 2000, 30, 1509; EP-A 841326, 1998, J. Chem. Soc. (C) 1967, 568; Synth. Commun. 2002, 32, 153; Pharm. Bull. 1955, 175; Heterocycles 1992, 33, 211; EP 462-452 1991, Liebigs Ann. 1981, 333; Monatsh. Chem. 1956, 87, 526; Chem. Pharm. Bull. 1987, 35, 3119; Pest. Man. Sci 2004, 60, 399; Synthesis 1984, 681; Heterocycles, 1994, 39, 345; Heterocycles, 1992, 33, 211; J. Chem. Soc., Perkin Trans. 1 1991, 2877; EP 301540 1989; Chem. Lett. 1984, 415.

The method of the invention for the preparation of the new azinylimidazoazines of structure (I) is carried out with a condensation agent. Water attracting chemicals are particularly suitable as condensation agents. These include preferably acid anhydrides and acid halides such as acetic anhydride, propionic anhydride, phosphorus(V) oxide (phosphorus pentoxide), phosphoryl chloride (phosphorus oxychloride), thionyl chloride, phosgene and diphosgene, in particular phosphoryl chloride.

The method of the invention for the preparation of compounds of the general structure (II) is carried out advantageously in the presence of a reaction auxiliary. All normal inorganic and organic bases are suitable. These include, for example, alkaline earth or alkaline metal hydrides, hydroxides, amides, alcoholates, acetates, carbonates or hydrogen carbonates, for example, sodium hydride, sodamide, sodium methylate, sodium ethylate, potassium tert.-butylate, sodium hydroxide, potassium hydroxide, ammonium hydroxide, sodium acetate, potassium acetate, calcium acetate, ammonium acetate, sodium carbonate, potassium carbonate, potassium hydrogen carbonate, sodium hydrogen carbonate or ammonium carbonate as well as tertiary amines, such as trimethylamine, triethylamine, tributylamine, N,N-dimethylaniline, pyridine, N-methylpiperidine, N,N-dimethylaminopyridine, diazabicyclooctane (DABCO), diazabicyclonone (DBN) or diazabicycloundecene (DBU)

The method of the invention for the preparation of the compounds of general structure (I) or (II) are optionally carried out with the use of one or more diluents. Such diluents are mainly inert organic solvents. These include especially aliphatic, alicyclic or aromatic, optionally halogenated hydrocarbons, for example petrol, benzene, toluene, xylene, chlorobenzene, dichlorobenzene, petroleum ether, hexane, cyclohexane, dichloromethane, chloroform and tetrachloromethane.

When carrying out the method of the invention for the preparation of compounds of general structure (I) and (II) the reaction temperature can be varied over a wide range. In general temperatures between 0° C. and 150° C., preferably between 10° C. and 120° C., are used.

The method of the invention is usually carried out at normal pressures. However, it is also possible to carry out the reaction of the invention at elevated or reduced pressures—in general between 0.1 bar and 10 bars.

To carry out the method of the invention the starting materials are generally used in approximately equimolar amounts. However, it is also possible to use one of the components in a larger excess. The conversion is generally carried out in a suitable diluent in the presence of a reaction auxiliary and the reaction mixture is generally stirred for several hours at the required temperature. Work-up is carried out with normal methods (cf. the preparation examples).

In a preferred embodiment of the method of the invention the starting materials of general structure (II) are prepared from the compounds of general structures (I) and (IV) by known methods with a reaction auxiliary and a diluent. After extensive concentration of the reaction mixture the residue is shaken with water and an organic solvent essentially immiscible with water. After adjusting the aqueous phase to an almost neutral value the organic phase is separated and after drying the solvent is removed under reduced pressure. The compounds of structure (II) thus obtained are then condensed into the compounds of structure (I) without further purification.

The compounds of the structure (I) and (II) obtainable by the aforementioned method can be converted into other compounds of structure (I) by normal methods with the scope of the above substituent definition.

For example, corresponding derivatives of structure (I), wherein X stands for halogen, are obtained from compounds of structure (I), where X stands for H (hydrogen), by reaction with suitable halogenating agents, for example chlorine, bromine, iodine, N-chlorosuccinimide, N-bromosuccinimide or N-iodosuccinimide in the presence of suitable diluents such as tetrachloromethane and/or acetonitrile at temperatures between −20° C. and +50° C. (cf. preparation examples).

For example, corresponding derivatives of structure (I), wherein X stands for formyl, are obtained from compounds of the structure (I), wherein X stands for H (hydrogen) by reaction with formylation agents, for example N,N-dimethylformamide, in the presence of phosphoryl chloride (POCl₃) at temperatures between −20° C. and +100° C. and subsequent work-up in the presence of aqueous ammonia (cf. preparation examples). By reaction with hydroxylamine or O-substituted hydroxylamines corresponding oximes or oxime ethers are obtained from the thus obtained formyl compounds of structure (I) (cf. preparation examples).

For example, derivatives of structure (I), wherein X stands for optionally substituted aryl, are obtained from compounds of the structure (I), wherein X stands for halogen, by reaction with corresponding arylboric acids in the presence of a reaction auxiliary, for example tetrakis(triphenylphosphine)palladium (Pd[P(C₆H₅)₃]₄) and sodium carbonate, in the presence of a diluent, for example toluene, and in an inert atmosphere (e.g. argon), at temperatures between 0° C. and 150° C. (“Suzuki crosscoupling”, cf. preparation examples).

For example, corresponding derivatives of structure (I), wherein X stands for optionally substituted arylethynyl, are obtained from compounds of structure (I), wherein X stands for halogen, by reaction with correspondingly substituted aryl acetylenes in the presence of a reaction auxiliary, for example bistriphenylphosphine)palladium(II) dichloride ([(PC₆H₅)₃]₂PdCl₂), copper(I) iodide (CuI) and triethylamine, in the presence of a diluent, for example tetrahydrofuran, and in an inert atmosphere (e.g argon), at temperatures between 0° C. and 50° C. (“Sonogashira crosscoupling”, cf. preparation examples).

Both the compounds of structure (I) and also of the structure (II), especially of the structure (II-b) are active compounds. For these active compounds it holds:

The active compounds, with good plant tolerance, favourable mammalian toxicity and good environmental tolerance, are suitable for the protection of plants and plant organs, for increasing crop yields, for improvement in the quality of the crop and for the control of zoopests, in particular arthropods such as insects and arachnids, nematodes which occur in agriculture, forestry, in gardens and leisure facilities, in storage and material protection as well as in the hygiene sector. They can preferably be used as plant protection agents. They are active against normal sensitive and resistant species as well as against all or individual development stages. The aforementioned pests include

from the order Isopoda e.g. Oniscus asellus, Armadillidium vulgare, Porcellio scaber;
from the order Diplopoda e.g. Blaniulus guttulatus;
from the order Chilopoda e.g. Geophilus carpophagus, Scutigera spp.;
from the order Symphyla e.g. Scutigerella immaculata;
from the order Thysanura e.g. Lepisma saccharina;
from the order Collembola e.g. Onychiurus armatus;
from the order Orthoptera e.g. Acheta domesticus, Gryllotalpa spp., Locusta migratoria migratorioides, Melanoplus spp., Schistocerca gregaria;
from the order Blattaria e.g. Blatta orientalis, Periplaneta americana, Leucophaea maderae, Blattella germanica;
from the order Dermaptera e.g. Forficula auricularia;
from the order Isoptera e.g. Reticulitermes spp.;
from the order Phthiraptera e.g. Pediculus humianus corporis, Haematopinus spp., Linognathus spp., Trichodectes spp., Damalinia spp.
from the order Thysanoptera e.g. Hercinothrips femoralis, Tjrips tabaci, Tjrips palmi, Frankliniella accidentalis;
from the order Heteroptera e.g. Eurygaster spp., Dysdercus intermedius, Piesma quadrata, Cimex lectularius, Rhodnius prolixus, Triatoma spp.;
from the order Homoptera e.g. Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp, Coccus spp.;
from the order Lepidoptera e.g. Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae;
from the order Coleoptera e.g. Anobium punctatum, Rhizopertha dominica, Bruchidius obtectus, Acanthoscelides obtectus, Hylotrupes bajulus, Agelastica alni, Leptinotarsa decemlineata, Phaedon cbchleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica, Lissorhoptrus oryzophilus;
from the order Hymenoptera e.g. Diprion spp., Hoplocampa spp., Lasius spp., Monomorium pharaonis, Vespa spp.
from the order Diptera e.g. Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp., Liriomyza spp.;
from the order Siphonaptera e.g. Xenopsylla cheopis, Ceratophyllus spp.;
the class Arachnida e.g. Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.;

The parasitic plant nematodes include e.g. Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

The compounds of the invention are used preferably for the control of sucking insects such as aphids (e.g. Aphis fabae, Aphis pomi, Aphis spiraecola, Aphis gossypii, Aphis nasturtii, Dysaphis plantaginea, Eriosoma spp., Rhopalosiphum padi, Acyrthosiphon pisum, Pemphigus bursarius, Myzus persicae, Myzus nicotianae, Myzus euphorbiae, Phylloxera spp. Toxoptera spp., Brevicoryne brassicae, Macrosiphum avenae, Macrosiphum euphorbiae, Nasonovia ribisnigri, Sitobion avenae, Brachycaudus helychrysii or Phorodon humuli), cicada (Idioscopis clypealis, Scaphoides titanus, Empoasca onuki, Empoasca vitis, Empoasca devastans, Empoasca libyca, Empoasca biguttula, Empoasca facialis or Erythroneura spp.), thrips (Hercinothrips femoralis, Scirtothrips aurantii, Scirtothrips dorsalis, Frankliniella schultzei, Frankliniella fusca, Frankliniella occidentalis, Frankliniella tritici, Kakothips spp., Thrips oryzae, Thrips palmi, Thrips tabaci), mealybugs (Pseudococcus spp., Planococcus spp., Phenacoccus spp.) or white fly (Aleurodes brassicae, Bemisia tabaci, Trialeurodes vaporariorum, Aleurodes proletella);

The compounds of the invention are not only active against plant, hygiene and storage pests but also against zoopests in the veterinary sector (ectoparasites) such as hard ticks, soft ticks, mange ticks, chigger mites, flies (stinging and licking), parasitic fly larvae, lice, biting mites, chewing mites and fleas. These parasites include:

from the order Anoplurida z.B. Haematopinus spp., Linognathus spp., Pediculus spp., Phtirus spp., Solenopotes spp.;
from the order Mallophagida and the suborders Amblycerina and Ischnocerina e.g. Trimenopon spp., Menopon spp., Trinoton spp., Bovicola spp., Werneckiella spp., Lepikentron spp., Damalina spp., Trichodectes spp., Felicola spp.;
from the order Diptera and the suborders Nematocerina and Brachycerina e.g. Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.;
from the order Siphonapterida z.B. Pulex spp., Ctenocephalides spp., Xenopsylla spp., Ceratophyllus spp.;
from the order Heteropterida z.B. Cimex spp., Triatoma spp., Rhodnius spp., Panstrongylus spp.;
from the order der Blattarida z.B. Blatta orientalis, Periplaneta americana, Blattela germanica, Supella spp.;
from the subclass Acari (Acarina) and the orders of the Meta- and Mesostigmata e.g. Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.;
from the order Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

Furthermore, protozoa such as Eimeria may be controlled.

The active compounds (I) and (II) and (II-b) of the invention are suitable for the control of arthropods that affect agricultural animals, for example, cattle, sheep, goats, horses, pigs, donkeys, camels, buffalo, rabbits, chickens, turkeys, ducks, geese, bees, other domestic animals, for example, dogs, cats, cage birds, aquarium fish and so-called experimental animals such as hamsters, guinea pigs, rats and mice. By control of these arthropods, death rates and deterioration in performance (in meat, milk, wool, skins, eggs, honey, etc) are lessened so that by the use of the active compounds of the invention more economic and simpler animal husbandry is possible.

The use of the active compounds of the invention in the veterinary sector is carried out in a known manner by enteral administration in the form of, for example, tablets, capsules, dips, drenches, granulates, pastes, boli, feed-through method, suppositories, by parenteral administration, for example by injection (intramuscular, subcutaneous, intravenous, intraperitoneal, etc.), implantation, by nasal administration, by dermal administration in the form of immersion or bathing (dipping), spray, pour-on and spot-on, washing, powdering as well as with the use of appliances containing the active compound, such as collars, ear markers, tail markers, limb bands, halters, marking devices, etc.

In applications for livestock, poultry, domestic animals, etc., the active compound of structure (I) or (II) or (II-b) can be applied as formulations (for example, powders, emulsions, free-flowing agents) which contain the active compound in general in amounts of 1 to 80% by weight, directly or after 100 to 10,000 times dilution, or use them as a chemical bath. In addition the compounds of the invention exhibit a high insecticidal action against insects that destroy technical materials.

As example and preferably—but not restricting—the following insects are named:

beetles, such as Hylotrupes bajulus, Chlorophorus pilosis, Anobium punctatum, Xestobium rufovillosum, Ptilinus pecticornis, Dendrobiurn pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;
hymenopteron, such as Sirex juvencus, Urocerus gigas, Urocerus gigas taignus, Urocerus augur;
termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;
Silverfish, such as Lepisma saccharina.

Technical materials within the present context are understood to be non-living materials such as preferably plastics, adhesives, glues, paper and cardboard, leather, wood, wood products and paints.

Most preferably the materials to be protected from insect infestation are wood and wood products.

Wood and wood products that can be protected by the agents of the invention or mixtures containing them are understood to be, for example,

building wood, wooden beams, railway sleepers, bridge components, landing stages, wooden vehicles, crates, palettes, containers, telephone masts, wood shuttering, wooden windows and doors, plywood, chipboard, joinery or wood products that are generally used in house building or in building joinery.

The active compounds can be used as such, in the form of concentrates or generally available formulations such as powder, granulates, solutions, suspensions, emulsions or pastes.

The formulations named can be prepared in the normal manner, for example by mixing the active compounds with at least one solvent or diluent, emulsifier, dispersing agent and/or bonding or fixing agent, water-repellent, optionally siccative and UV stabilisers and optionally colours and pigments as well as other processing auxiliaries.

The insecticidal agent or concentrate used for the protection of wood or wood products generally contain the active compound of the invention in a concentration of 0.0001 to 95% by weight, especially, 0.001 to 60% by weight.

The amount of agent or concentrate used is dependent upon the species and incidence of the insects and of the medium. The optimal amount used in the application can be determined by the use of test series. In general, however, it is sufficient to use 0.0001 to 20% by weight, preferably 0.001 to 10% by weight of the active compound relative to the material to be protected.

Suitable solvents or diluents are organic solvents or solvent mixtures and/or oily or oil-like, poorly volatile organic solvents or solvent mixtures and/or a polar organic solvent or solvent mixture and/or water and optionally an emulsifier and/or wetting agent.

In addition protozoa such as Eimeroa can be controlled.

The compounds of the invention exhibit a strong microbicidal action and can be used in plant protection and material protection for the control of micro-organisms such as fungi and bacteria

Fungicides may be used in plant protection for the control of Plasmodiophoromycetes, Oomycetes, Chytridiomycetes, Zygomycetes, Ascomycetes, Basidiomycetes and Deuteromycetes.

Bactericides may be used in plant protection for the control of Pseudomonadaceae, Rhizobiaceae, Enterobacteriaceae, Corynebacteriaceae and Streptomycetaceae.

For example but not restricting are a number of pathogens of fungal and bacterial diseases which come within the above listed generic heading are named:

Xanthomonas species, for example Xanthomonas campestris pv. oryzae;
Pseudomonas species, for example Pseudomonas syringae pv. lachrymans;
Erwinia species, for example Erwinia amylovora;
Pythium species, for example Pythium ultimum;
Phytophthora species, for example Phytophthora infestans;
Pseudoperonospora species, for example Pseudoperonospora humuli or Pseudoperonospora cubensis;
Plasmopara species, for example Plasmopara viticola;
Bremia species, for example Bremia lactucae;
Peronospora species, for example Peronospora pisi or P. brassicae;
Erysiphe species, for example Erysiphe graminis;
Sphaerotheca species, for example Sphaerotheca fuliginea;
Podosphaera species, for example Podosphaera leucotricha;
Venturia species, for example Venturia inaequalis;
Pyrenophora species, for example Pyrenophora teres or P. graminea
(conidial form: Drechslera, Syn: Helminthosporium);
Cochliobolus species, for example Cochliobolus sativus
(conidial form: Drechslera, Syn: Helminthosporium);
Uromyces species, for example Uromyces appendiculatus;
Puccinia species, for example Puccinia recondita;
Sclerotinia species, for example Sclerotinia sclerotiorum;
Tilletia species; for example Tilletia caries;
Ustilago species, for example Ustilago nuda or Ustilago avenae;
Pellicularia species, for example Pellicularia sasakii;
Pyricularia species, for example Pyricularia oryzae;
Fusarium species, for example Fusarium culmorum;
Botrytis species, for example Botrytis cinerea;
Septoria species, for example Septoria nodorum;
Leptosphaeria species, for example Leptosphaeria nodorum;
Cercospora-Arten, for example Cercospora canescens;
Alternaria species, for example Alternaria brassicae;
Pseudocercosporella species, for example Pseudocercosporella herpotrichoides.

The active compounds of the invention also exhibit a strong fortifying action in plants. They are suitable, therefore, for the mobilisation of the plants intrinsic resistance to infestation by undesirable micro-organisms.

Plant fortifying (resistance inducing) substances in the present context are understood to be such substances that are able to stimulate the resistance of plants so that the treated plants after subsequent inocculation with undesirable micro-organisms develop far-reaching resistance towards these micro-organisms.

Adverse micro-organisms in this connection are understood to be phytopathogenic fungi, bacteria and viruses. The substances of the invention can thus be used to protect the plants over a certain period after treatment against infestation by the named pathogens. The time period during which their protection is induced generally lasts for 1 to 10 days, preferably 1 to 7 days after the treatment of the plants with the active compounds.

The good plant tolerance of the active compounds in the concentrations necessary for control of plant diseases makes treatment of the visible plant parts, plant and seed stock and the ground possible.

The active compounds of the invention can be used with particular success in the control of cereal diseases such as Ustilago avenae.

The active compounds of the invention are also suitable for increasing crop yields; They are also of low toxicity and exhibit good plant tolerance.

The active compounds of the invention can also optionally be used in certain concentrations and application amounts as herbicides and for influencing plant growth. They may also be optionally used as intermediates and precursors in the synthesis of further active compounds.

According to the invention all plants and plant parts can be treated. Plants are hereby understood to mean all plants and plant populations such as desirable and undesirable wild plants or cultigens (including naturally occurring cultigens). Cultigens can be plants that can be obtained by conventional breeding and optimisation methods or by biotechnology or genetic engineering methods or combinations of these methods, including transgenic plants and including plant varieties that are protectable or not protectable by plant varieties protection rights. Plant parts are understood to be all above ground and below ground parts and organs of the plants such as scion, leaf, blossom and root, including, for example, leaves, needles, stalks, stems, blossoms, fruiting bodies, fruits and seed as well as roots, bulbs, and rhisomes. Crops as well as vegetative and generative reproduction material, for example scions, bulbs, rhizomes, shoots and seed also belong to plant parts.

The treatment according to the invention of plants and plant parts with the active compound can be carried out directly or by action on their environment, habitat or store by means of the normal treatment methods, for example, by immersion, spraying, evaporation, misting, scattering, painting, injecting, and with reproductive material, in particular with seed, also by single or multiple jacketing.

In material protection the substances of the invention may be used for the protection of technical materials against infestation and destruction by undesirable micro-organisms.

Technical materials are understood to be in the present context non-living materials that have been prepared for use in engineering. For example, technical materials that are to be protected against micro-biological change or destruction by the active materials of the invention can be adhesives, glues, paper and cardboard, textiles, leather, wood, paint and plastic articles, cooling lubricants and other materials that can be infested or destroyed by micro-organisms. Within the context of materials to be protected are also parts of production plants, for example cooling circuits, which can be adversely affected by the propagation of micro-organisms. Within the context of the present invention, preferably mentioned as technical materials are adhesives, glues, paper and cardboard, leather, wood, paints, cooling lubricants and heat exchanger liquids, particularly preferred is wood.

Micro-organisms that can cause the degradation or alteration of technical materials are for example bacteria, fungi, yeasts, algae and moulds. The active compounds of the invention act against preferably fungi, especially mould fungi, fungi that discolour and destroy wood (Basidiomyceten), and against slime organisms and algae.

As examples micro-organisms of the following genus are named:

Alternaria, such as Alternaria tenuis;
Aspergillus, such as Aspergillus niger;
Chaetomium, such as Chaetomium globosum;
Coniophora, such as Coniophora puetana;
Lentinus, such as Lentinus tigrinus;
Penicillium, such as Penicillium glaucum;
Polyporus, such as Polyporus versicolor;
Aureobasidium, such as Aureobasidium pullulans;
Sclerophoma, such as Sclerophoma pityophila;
Trichoderma, such as Trichoderma viride;
Escherichia, such as Escherichia coli;
Pseudomonas, such as Pseudomonas aeruginosa;
Staphylococcus, such as Staphylococcus aureus

Depending upon their respective physical and/or chemical properties the active compounds can be converted into the usual formulations such as solutions, emulsions, suspensions, powders, foams, pastes, granulates, aerosols, micro-encapsulation in polymeric materials and in jackets for seed, as well as in ULV cold and warm fogging formulations.

These formulations can be prepared in the normal manner, for example by mixing the active compounds with diluents, that is liquid solvents, pressurised liquid gases and/or solid supports, optionally with the use of surfactants, that is emulsifiers and/or dispersants and/or foaming agents. Where water is used as diluent organic solvents can also be used, for example, as auxiliary solvents. Suitable liquid solvents are essentially: aromatics, such as xylene, toluene or alkylnaphthalines, chlorinated aromatics or chlorinated aliphatic hydrocarbons, such as chlorobenzene, chloroethylene or methylene chloride, aliphatic hydrocarbons, such as cyclohexane or paraffins, e.g. natural oil fractions, alcohols, such as butanol or glycol as well as their ethers and esters, ketones, such as acetone, methylethylketone, methylisobutylketone or cyclohexanone, highly polar solvents, such as dimethylformamide and dimethylsulphoxide, as well as water. By liquid gas diluents or supports are meant such liquids that are gaseous at normal temperatures and under normal pressure, e.g. aerosol propellants, such as halohydrocarbons as well as butane, propane, nitrogen and carbon dioxide. Suitable solid supports are: e.g. natural mineral powders, such as kaolin, argillaceous earth, chalk, quartz, attapulgite, montmorillonite or diatomaceous earth and synthetic mineral powders such as highly dispersed silica, aluminium oxide and silicates. Suitable solid supports for granulates are: e.g. broken and fractionated natural stone such as calcite, marble, pumice, sepiolite, dolomite as well as synthetic granulates of inorganic and organic flours as well as granulates of organic materials like wood flour, coconut husks, maize cobs and tobacco stalks. Suitable emulsifiers and/or foaming agents are: e.g. nonionogenic and anionic emulsifiers such as polyoxyethylene-fatty acid esters, polyoxyethylene-fatty alcohol ethers, e.g. alkylarylpolyglycol ether, alkyl sulphonates, alkyl sulphates, aryl sulphonates as well as protein hydrolysates. Suitable dispersants are: e.g. lignin-sulphite waste liquour and methylcelluloses.

Deposit builders such as carboxymethylcellulose, natural and synthetic powdery, granular or latex-like polymers such as gum arabic, polyvinylalcohol, polyvinylacetate, as well as natural phospholipids such as cephalins and lecithins, and synthetic phospholipids can be used in the formulations. Further additives can be mineral and vegetable oil.

Colourants such as inorganic pigments, e.g iron oxide, titanium oxide, ferrocyan blue, and organic colourants such as alizarin, azo and metallphthalocyanin dyes, and trace nutrients such as iron, manganese, boron, copper, cobalt, molybdenum and zinc salts can be used

Usually the formulations contain between 0.1 and 95 percent by weight of the active compound, preferably between 0.5 and 90%.

The active materials of the invention can be used as such or in their formulations also in admixture with known fungicides, bactericides, acaricides, nematocides, or insecticides in order, for example, to broaden the spectrum of activity or to avoid the development of resistance. In many cases synergetic effects are obtained, that is the activity of the mixture is greater than the activity of the individual compounds.

The following compounds are suitable as mixing partner:

### Fungicides:

2-phenylphenol; 8-hydroxyquinoline sulphate; acibenzolar-S-methyl; aldimorph, amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; quinine methionate; chlobenthiazone; chlorfenazole; chloroneb; chlorothalonil; chlozolinate; clozylacon; cyazofamid; cyflufenamid; cymoxanil; cyproconazole; cyprodinil; cyprofuram; dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-A1; fosetyl-sodium; fuberidazole; furalaxyl; furametpyr; furcarbanil; furmecyclox; guazatine; hexachlorobenzene; hexaconazole; hymexazol; imazalil; imibenconazole; iminoctadine triacetate; iminoctadine tris(albesil; iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulphonyl)amino]-butanamide; 1-(1-naphthalenyl)-1H-pyrrole-2,5-dione; 2,3,5,6-tetrachloro-4-(methylsulphonyl)pyridine; 2-amino-4-methyl-N-phenyl-5-thiazole carboxamide; 2-chloro-N-2,3-dihydro-1,1,3-trimethyl-1H-inden-4-yl)-3-pyridine carboxamide; 3,4,5-trichloro-2,6-pyridinedicarbonitrile; actinovate; cis-1-(4-chlorophenyl)-2-(1H-1,2,4-triazole-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropane carboxamide; N-butyl-8-(1,1-dimethylethyl)-1-oxaspiro[4.5]decan-3-amine; sodium tetrathiocarbonate;
as well as copper salts and preparations such as Bordeaux mixture; copper hydroxide; copper naphthenate; copper oxychloride; copper sulphate; cufraneb; cuprous oxide; mancopper; oxine-copper.

### Bactericides:

bronopol, dichlorophen, nitrapyrin, nickel dimethyldithiocarbamate, kasugamycin, octhilinon, furan carboxylic acid, oxytetracycline, probenazol, streptomycin, tecloftalam, copper sulphate and other copper preparations.

### Insecticides/Acaricides/Nematicides:

abamectin, ABG-9008, acephate, acequinocyl, acetamiprid, acetoprole, acrinathrin, AKD-1022, AKD-3059, AKD-3088, alanycarb, aldicarb, aldoxycarb, allethrin, alpha-cypermethrin (alpha-methrin), amidoflumet, aminocarb, amitraz, avermectin, AZ-60541, azadirachtin, azamethiphos, azinphos-methyl, azinphos-ethyl, azocyclotin,
Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, Baculoviren, Beauveria bassiana, Beauveria tenella, benclothiaz, bendiocarb, benfuracarb, bensultap, benzoximate, beta-cyfluthrin, beta-cypermethrin, bifenazate, bifenthrin, binapacryl, bioallethrin, bioallethrin-S-cyclopentyl isomer, bioethanomethrin, biopermethrin, bioresmethrin, bistrifluoron, BPMC, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben,
cadusafos, bamphechlor, carbaryl, carbofuran, carbophenothion, carbosulfan, cartap, CGA-50439, quinine methionate, chlordane, chlordimeform, chloethocarb, chlorethoxyfos, bhlorfenapyr, chlorfenvinphos, chlorfluazuron, chiormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, Cydia pomonella, cyfluthrin, cyhalothrin, cyhexatin, cypermethrin, cyphenothrin (1R-trans isomer), cyromazine,
DDT, deltamethrin, demeton-S-methyl, demeton-S-methylsulphone, diafenthiuron, dialifos, diazinon, dichlofenthion, dichlorvos, dicofol, dicrotophos, dicyclanil, diflubenzuron, dimefluthrin, dimethoate, dimethylvinphos, dinobuton, dinocap, dinotefuran, diofenolan, disulfoton, docusatsodium, dofenapyn, DOWCO-439,
eflusilanate, emamectin, emamectin-benzoate, empenthrin (1R isomer), endosulfan, Entomopthora spp., EPN, esfenvalerate, ethiofencarb, ethiprole, ethion, ethoprophos, etofenprox, etoxazole, etrimfos,
famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb,
gamma-cyhalothrin, gamma-HCH, gossyplure, grandlure, granuloseviren,
halfenprox, halofenozide, HCH, HCN-801, heptenophos, hexaflumuron, hexythiazox, hydramethynone, hydroprene,
IKA-2002, imidacloprid, imiprothrin, indoxacarb, iodofenphos, iprobenfos, isazofos, isofenphos, isoprocarb, isoxathion, ivermectin,
japonilure,
kadethrin, kernpolyederviren, kinoprene,
lambda-cyhalothrin, lindane, lufenuron,
malathione, mecarbam, mesulfenfos, metaldehyde, metam-sodium, methacrifos, methamidophos, Metharhizium anisopliae, Metharhizium flavoviride, methidathion, methiocarb, methomyl, methoprene, methoxychlor, methoxyfenozide, metofluthrin, metolcarb, metoxadiazone, mevinphos, milbemectin, milbemycin, MKI-245, MON-45700, monocrotophos, moxidectin, MTI-800,
naled, NC-104, NC-170, NC-184, NC-194, NC-196, niclosamide, Nicotine, nitenpyram, nithiazine, NNI-0001, NNI-0101, NNI-0250, NNI-9768, novaluron, noviflumuron,
OK-5101, OK-5201, OK-9601, OK-9602, OK-9701, OK-9802, omethoate, oxamyl, oxydemeton-methyl,
Paecilomyces fumosoroseus, parathion-methyl, parathion (-ethyl), permethrin (cis, trans), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphos-ethyl, potassium oleate, prallethrin, profenofos, profluthrin, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen,
quinalphos,
resmethrin, RH-5849, ribavirin, RU-12457, RU-15525,
S-421, S-1833, salithion, sebufos, SI-0009, silafluofen, spinosad, spirodiclofen, spiromesifen, sulfluramid, sulfotep, sulprofos, SZI-121,
tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R isomer), tetrasul, theta-cypermethrin, thiacloprid, thiamethoxam, thiapronil, thiatriphos, thiocyclam hydrogen oxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, Trichoderma atroviride, triflumuron, trimethacarb,
vamidothion, vaniliprole, verbutin, Verticillium lecanii,

### WL-108477, WL-40027,

### YI-5201, YI-5301, YI-5302,

XMC, xylylcarb,
ZA-3274, zeta-cypermethrin, zolaprofos, ZXI-8901,
the compound 3-methylphenylpropylcarbamate (tsumacide Z),
the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo isomers (CAS Reg. No. 185984-60-5) (cf. WO-96/37494, WO-98/25923),
as well as preparations that contain insecticidally active plant extracts, nematodes, fungi or viruses.

Also a mixture with other known active compounds such as herbicides, or with fertilizers and growth regulators, safeners or semicochemicals is possible.

Moreover, the compounds of structure (I) or (II) or (II-b) of the invention also exhibit very good antimycotic activities. They possess a very broad antimycotic spectrum of activity, especially against dermatophytes and yeasts, mould and biphasic fungi (e.g. against Candida species such as Candida albicans, Candida glabrata) as well as Epidermophyton floccosum, Aspergillus species such as Aspergillus niger and Aspergillus funigatus, Trichophyton species such as Trichophyton mentagrophytes, Microsporon species such as Microsporon canis and audouinii. The listing of the fungi in no way represents a restriction of the recordable mycotic spectrum, but has only illustrative character.

The active compounds can be used as such, in the form of their formulations or application forms prepared from them such as ready-to-use solutions, suspensions, powder sprays, pastes, soluble powders, dusting agents and granulates. Application is carried out in a normal manner, for example by pouring, spraying or sprinkling, scattering, dusting, foaming, brushing, etc. It is also possible to deploy the active compounds with the ultra-low volume method or to inject the active compound preparation or the active compound itself into the ground. The seed of the plants can also be treated.

During the use of the active compounds of the invention as fungicides the amount applied can be varied over a wide range according to the method of application. In the treatment of plant parts the amount of active compound applied generally lies between 0.1 and 10,000 g/ha, preferably between 10 and 1,000 g/ha. In seed treatment the amounts of active compound generally lie between 0.001 and 50 g per kilogram of seed, preferably between 0.01 and 10 g per kilogram of seed. In the treatment of the ground the amount of active compound applied generally lies between 0.1 and 10,000 g/ha, preferably between 1 and 5,000 g/ha.

Furthermore the active compounds of the invention can be present during use as insecticides in their normal commercial formulations as well as in forms of application prepared from these formulations in admixture with synergists. Synergists are compounds through which the activity of the active compound is increased without the synergist added itself having to be active.

The active compounds of the invention can also be present during use as insecticides in their normal commercial formulations as well as in forms of application prepared from these formulations in admixture with inhibitors which reduce the degradation of the active compound after application in the environment of the plants, on the surface of the plant parts or in the plant tissues.

The active compound content of the application forms prepared from the normal commercial formulations can be varied over a wide range. The active material concentration of the application forms can lie between 0.0000001 and 95% by weight, preferably between 0.0001 and 1% by weight.

The application takes place in a normal manner adapted to the application form.

During application against hygiene and storage pests the active compound is characterised by an excellent residual action on wood and clay as well as by a good alkali stability on limed foundations.

As already described, according to the invention all plants and their parts can be treated. In a preferred embodiment wild or plant species and plant varieties obtained by conventional biological breeding methods such as crossing or protoplast infusion and their parts are treated. In a further preferred embodiment transgenic plants and plant varieties which were produced by genetic engineering methods optionally in combination with conventional methods (genetic modified organisms) and their parts are treated. The terms “parts” and “parts of plants” or “plant parts” were explained above.

Especially preferred according to the invention plants of the respective normal commercial or customarily used plant varieties are treated. Plant varieties are understood to mean plants with new properties (“traits”) that have been bred by conventional breeding, by mutagenesis or by recombinant DNA techniques. These can be varieties, strains, bio- and genotypes.

Depending upon the plant species or plant varieties, their position and growth conditions (soil, climate, vegetation period, nutrition), superadditive (“synergistic”) effects can occur by the treatment of the invention. Thus, for example, lower amounts of application and/or widening of the activity spectrum and/or increase in the action of the substances and agents that may be used according to the invention, improved plant growth, increased tolerance towards high or low temperatures, increase tolerance towards drought or towards water or soil salt content, increased blossoming performance, simplified harvesting, acceleration in ripening, increased harvest yields, higher quality and/or nutritional value of the harvested product, better storage life and/or processing of the harvested product are possible which extend beyond actually the expected effects.

All plants that receive by genetic engineering modification genetic material that imparts particularly advantageous valuable properties (“traits”) to these plants belong to the transgenic (obtained by genetic engineering) plants or plant varieties to be preferably treated in accordance with the invention. Examples of such properties are improved plant growth, increased tolerance toward high or low temperatures, increased tolerance toward drought or toward water or soil salt content, improved blossoming performance, simplified harvesting, accelerated ripening, increased harvest yields, improved quality and/or nutritional value of the crop, better storage life and/or processing of the crop. Further and particularly emphasised examples of such properties are increased resistance of the plants toward zoopest and microbial pests, such as toward insects, mites, pathogenic plant fungi, bacteria and/or viruses as well as an increased tolerance of the plants toward certain herbicides. Examples of such transgenic plants are the important cultigens such as cereals (wheat, rice), maize, soy, potato, cotton, tobacco, rape as well as fruit plants (with the fruits apple, pear, citrus fruits and grapes), whereby maize, soy, potato, cotton, tobacco and rape are especially emphasised. Properties (“traits”) especially emphasised are the increased tolerance of the plants toward insects, arachnids, nematodes and slugs through the toxins formed in the plants, especially those that are produced in the plants (hereinafter known as “Bt plants”) by the genetic material from Bacillus Thuringiensis (e.g. from the genes CryIA(a), CryIA(b), CryIA(c), CryIIA, CryIIIA, CryIIIB2, Cry9c Cry2Ab, Cry3Bb and CryIF as well as their combinations). Also particularly emphasised as properties (“traits”) is the increased resistance of plants toward fungi, bacteria and viruses through systemically acquired resistance (SAR), systemin, phytoalexine, elicitors and resistance genes and correspondingly expressed proteins and toxins. Further particularly emphasised properties (“traits”) are the increased tolerance of the plants to certain active herbicidal compounds, for example imidazolines, sulphonyl ureas, glyphosate or phosphinotricin (e.g. “PAT”-gene). The respective genes imparting the desired properties (“traits”) can also occur in the transgenic plants in combination with each other. Examples of such “Bt plants” are maize varieties, cotton varieties, soy varieties and potato varieties that are marketed under the trade marks YIELD GARD® (e.g. maize, cotton, soy), KnockOut® (e.g. maize), StarLink® (e.g. maize), Bollgard® (cotton), Nucotn® (cotton) and NewLeaf® (potato). Examples of herbicide tolerant plants are maize varieties, cotton varieties and soy varieties that are marketed under the trade marks Roundup Ready® (tolerance towards glyphosate, e.g. maize, cotton, soy), Liberty Link® (tolerance toward phosphinotricin, e.g. rape), IMI® (tolerance toward imidazolinones) and STS® (tolerance toward sulphonyl ureas, e.g. maize). Also mentioned a herbicide resistance (conventionally bred for herbicide tolerance) plants are those varieties marketed under the name Clearfield® (e.g. maize). Naturally these statements also apply to plant varieties developed or marketed in the future with these genetic properties (“traits”) or those developed in the future.

According to the invention the plants described can be particularly advantageously treated with the compounds of general structure (I) or (II) or (II-b) or active compound mixtures of the invention. The preferred ranges described above for the active compounds or mixtures hold also for the treatment of these plants. Particularly mentioned is plant treatment with the compounds or mixtures specially described in the present text.

In addition to the lethal action on pests the compounds of structure (I) or (II) or (II-b) or their salts are also characterised by a pronounced repellent effect.

Repellent within the meaning of the description is a substance or substance mixture which acts in a repellent or dispelling manner on other organisms, especially pests or parasites. The term also includes effects such as the anti-feeding effect where nutrient uptake is destroyed or impaired (feeding repellent effect), suppression of egg laying or an effect upon population development. Subject matter of the invention is therefore the use of the compounds of structure (I) or (II) or (II-b) or their salts to achieve the named effects, especially with the pests named in the biological examples.

Subject matter of the invention is also a method for the resistance to or the repulsion of pests whereby one or more of the compounds of structure (I) or (II) or (II-b) or their salts are applied at the site where pests are to be excluded or repelled.

Application in the case of a plant can mean, for example, the treatment of the plant or also the seed.

Where the effect upon populations is concerned it is of interest that the effects can also be observed sequentially in the development of a population, when they can be additive. Thus although the individual effect itself can have only a level of activity significantly below 100%, overall at the end a 100% action is still achieved.

In addition the compounds of structure (I) or (II) or (II-b) or their salts are characterised in that if the effects described above are to be exploited the agent can be applied at an earlier time point than is usual in direct control. The effect is frequently long-lasting so that a duration of action of more than 2 months is achieved.

The effect occurs in insects, arachnids and the others of the above described pests.

The following examples serve to illustrate the invention.

### PREPARATION EXAMPLES

### Example 1

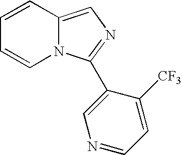

2.90 g (10.3 mMol) N-(pyridin-2-ylmethyl)-4-trifluoromethyl-nicotinamide were mixed with 30 ml phosphoryl chloride (POCl₃) with ice cooling and the mixture was stirred for 10 hours at 100° C. The mixture was then poured onto about five times the volume of ice, made weakly alkaline with conc. aqueous ammonia and extracted with methylene chloride. The organic phase was washed with water, dried with sodium sulphate and filtered. The solvent was carefully distilled from the filtrate under reduced pressure and the residue was worked up by column chromatography (silica gel, methylene chloride/acetonitrile, vol.: 7:3).

1.0 g (37% of theory) 3-(4-trifluoromethylpyridin-3-yl)-imidazo[1,5-a]pyridine was obtained

¹H NMR (400 MHz, CDCl₃, 6, ppm), 8.94 (d, 1H), 8.86 (s, 1H), 7.76 (d, 1H), 7.64 (s, 1H), 7.59 (d, 1H), 7.53 (d, 1H), 6.78 (dd, 1H), 6.55 (dd, 3H).

### Example 2

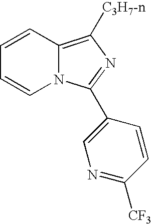

### (Inclusion of Precursor Synthesis)

294 mg (2.63 mMol) 1-(pyridin-2-yl)butylamine and 0.40 ml (2.86 mMol) triethylamine were dissolved in 10 ml methylene chloride and stirred for 5 minutes at room temperature (ca. 20° C.). Then with further stirring a solution of 500 mg (2.39 mMol) 6-trifluoromethylpyridine-3-carbonyl chloride in 10 ml methylene chloride were added dropwise and the reaction mixture stirred for two hours at room temperature. The reaction mixture was then diluted to twice the volume with methylene chloride, washed with aqueous potassium hydrogen sulphate solution, dried with magnesium sulphate and filtered. The solvent was carefully distilled from the filtrate under reduced pressure.

The crude residue thus obtained containing N-1-pyridine-2-ylbutyl)-6-trifluoromethyl-nicotinamide—90 mg—was taken up into 5 ml phosphoryl chloride POCl₃), the mixture stirred for three hours at 10° C., then poured onto about five times the volume of ice, made weakly alkaline with conc. aqueous ammonia and then extracted with ethyl acetate (3×50 ml). The combined organic phases were dried with magnesium sulphate and filtered. The solvent was carefully distilled from the filtrate under reduced pressure and the residue worked up by column chromatography (silica gel, methylene chloride/acetonitrile, vol.: 7:3).

14 mg (16% of theory) 1-n-propyl-3-(6-trifluoromethyl)pyridin-3-yl)-imidazo[1,5-a]pyridine were obtained.

¹H NMR (400 MHz, CDCl₃, δ, ppm): 9.17 (d, 1H), 8.34 (dd, 1H), 8.21 (d, 1H), 7.78 (d, 1H), 7.46 (d, 1H), 6.72 (dd, 1H), 6.63 (dd, 1H), 2.90 (t, 2H), 1.77 (sext, 2H), 1.02 (t, 3H). MS (CI), m/z 334 (M⁺+2, 55), 332 (M⁺, 100), 300 (20), 298 (60).

### Example 3

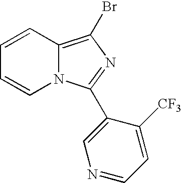

### (Subsequent Transformation)

0.50 g (1.90 mMol) 3-(4-trifluoromethylpyridin-3-yl)-imidazo[1,5-a]pyridine (cf. example 1) were dissolved in 45 ml tetrachloromethane and cooled to 5° C. Then a solution of 334 mg (2.09 mMol) bromine in 10 ml tetrachloromethane were added dropwise with stirring and the reaction mixture was stirred for a further five minutes at 5° C. The solvent was then distilled off under reduced pressure and the residue was taken up into dichloromethane/water and neutralised with aqueous sodium hydroxide. The organic phase was separated and the aqueous phase was further extracted with dichloromethane (3×100 ml). The combined organic solutions were dried with magnesium sulphate and filtered. The solvent was carefully distilled from the filtrate under-reduced pressure

0.60 g (92% of theory) 1-bromo-3-(4-trifluoromethylpyridin-3-yl)-imidazo[1,5-a]pyridine were obtained as amorphous residue.

¹H NMR (400 MHz, CDCl₃, δ, ppm): 8.96 (d, 1H), 8.85 (s, 1H), 7.76 (d, 1H), 7.57 (d, 1H), 7.48 (d, 1H), 6.86 (dd, 1H), 6.62 (dd, 1H); ¹⁹F NMR (300 MHz, CDCl₃)-62.66 (CF₃); MS (CI), m/z 344 (M⁺+2, 40), 342 (M⁺, 44), 264 (100).

### Example 4

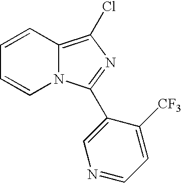

(Subsequent transformation))

0.30 g (1.14 mMol) 3-(4-trifluoromethylpyridin-3-yl)-imidazo[1,5-a]pyridine (cf. example 1) were taken up in 15 ml acetonitrile and treated at room temperature (ca. 20° C.) with 152 mg (1.14 mMol) N-chlorosuccinimide. The reaction mixture was stirred for four days at room temperature, then the mixture was added to double the volume of water and extracted with ethyl acetate (3×50 ml). The combined organic solutions were dried with magnesium sulphate and filtered. The solvent was carefully distilled from the filtrate under reduced pressure.

285 mg (84% of theory) 1-chloro-3-(4-trifluoromethylpyridin-3-yl)-imidazo[1,5-a]pyridine were obtained as amorphous residue.

¹H NMR (400 MHz, CDCl₃, δ, ppm): 8.95 (d, 1H), 8.85 (s, 1H), 7.78 (d, 1H), 7.56 (d, 1H), 7.41 (d, 1H), 6.85 (dd, 1H), 6.63 (dd, 1H).

### Example 5

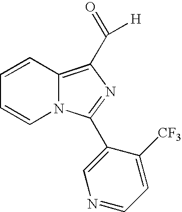

### (Subsequent Transformation)

0.50 g (1.90 mMol) 3-(4-trifluoromethylpyridin-3-yl)-imidazo[1,5-a]pyridine (cf. example 1) were taken up in 45 ml N,N-dimethylformamide and cooled to 0° C. Then 0.89 ml phosphoryl chloride (POCl₃) were added, the mixture warmed slowly to 60° C. and stirred at this temperature for one hour. After cooling the mixture to room temperature it was diluted to about twice the volume with water and made weakly alkaline with aqueous ammonia. It was then extracted with methylene chloride (3×50 ml). The combined organic extracts were dried with magnesium sulphate and filtered. The solvent was carefully distilled from the filtrate under reduce pressure.

0.51 g (92% of theory) 3-(4-trifluoromethylpyridin-3-yl)-imidazo[1,5-a]pyridine-1-carboxaldehyde were obtained as amorphous residue.

¹H NMR (400 MHz, CDCl₃, δ, ppm): 10.32 (s, 1H), 9.04 (d, 1H), 8.88 (s, 1H), 8.38 (d, 1H), 7.78 (d, 1H), 7.69 (d, 1H), 7.32 (dd, 1H), 6.90 (dd, 1H); ¹³C-NMR (100 MHz, CDCl₃), MS: 186.2, 153.2, 152.6, 138.8, 134.0, 132.8, 131.3, 126.7, 122.1, 122.0, 120.5, 120.4, 119.9, 116.0; MS (CI), m/z 292 (M⁺+1, 100).

### Example 6

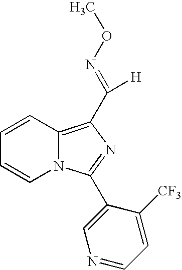

### (Subsequent Transformation)

0.10 g (0.34 mMol) 3-(4-trifluoromethylpyridin-3-yl)-imidazo[1,5-a]pyridine-1-carboxaldehyde were taken up in 6 ml methanol and this solution was added dropwise at room temperature (ca. 20° C.) to a solution of 34 mg (0.41 mMol) O-methylhydroxylamine hydrochloride and 34 mg (0.41 mMol) sodium acetate in 4 ml methanol. The reaction mixture was stirred for an hour at room temperature and the evaporated under reduced pressure. The residue was taken up into ethyl acetate/water, the aqueous phase was separated and extracted with ethyl acetate (3×50 ml). The combined organic extracts were dried with magnesium sulphate and filtered. The solvent was carefully distilled from the filtrate under reduced pressure.

113 mg (92% of theory) 1-methoximinomethyl-3-(4-trifluoromethylpyridin-3-yl)-imidazo[1,5-a]-pyridine were obtained as amorphous residue.

¹H NMR (400 MHz, CDCl₃, δ, ppm): 8.98 (d, 1H), 8.86 (s, 1H), 8.42 (s, 1H), 8.06 (d, 1H), 7.74 (d, 1H), 7.57 (d, 1H), 6.96 (dd, 1H), 6.69 (dd, 1H); MS (CI), m/z 321 (M⁺+1, 100), 289 (50).

### Example 7

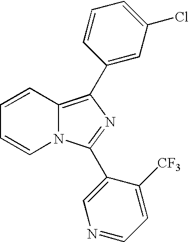

### (Subsequent Transformation)

A mixture of 150 mg (0.44 mMol) 1-bromo-3-(4-trifluoromethylpyridin-3-yl)-imidazo[1,5-a]-pyridine, 75 mg (0.48 mMol) 3-chlorophenylboric acid, 26 mg (0.02 mMol) tetrakis(triphenylphosphine)palladium (Pd[P(C₆H₅)₃]₄) and 5 ml toluene was stirred under argon and treated with 0.6 ml of a 2-molar aqueous sodium carbonate solution. The reaction mixture was heated for 6 hours under reflux and extracted with water/ethyl acetate after cooling to room temperature. The aqueous phase was then extracted with ethyl acetate (3×50 ml), the combined organic phases dried with magnesium sulphate and filtered. The filtrate was evaporated under reduced pressure and the residue was worked up by column chromatography (silica gel, ethyl acetate).

62 mg (36% of theory) 1-(3-chlorophenyl)-3-(4-trifluoromethylpyridin-3-yl)-imidazo[1,5-a]-pyridine were obtained.

¹H NMR (400 MHz, CDCl₃, 6, ppm): 8.98 (d, 1H), 8.95 (s, 1H), 7.88 (m, 2H), 7.82 (s, 1H), 7.78 (d, 1H), 7.59 (d, 1H), 7.38 (dd, 1H), 7.30 (m, 1H), 6.92 (dd, 1H), 6.63 (dd, 1H).

### Example 8

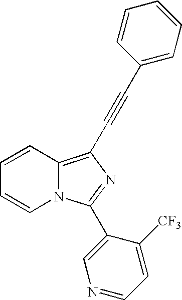

### (Subsequent Transformation)

A mixture of 13 mg bis(triphenylphosphine)palladium dichloride (Pd[P(C₆H₅)₃]Cl₂) (0.02 mMol), 7 mg (0.04 mMol) copper(I) iodide, 4 ml triethylamine and 4 ml tetrahydrofuran was stirred under argon for 5 minutes at room temperature (ca. 20° C.). Then, with further stirring, a solution of 140 mg (0.36 mMol) 1-iodo-3-(4-trifluoromethylpyridin-3-yl)-imidazo[1,5-a]pyridine in 1 ml tetrahydrofuran was added and the mixture stirred for 25 minutes at room temperature. 37 mg (0.36 mMol) phenylacetylene were then added and the reaction mixture was stirred a further 8 hours at room temperature. It was then extracted with water/ethyl acetate and the aqueous phase extracted further with ethyl acetate (3×50 ml). The combined organic phases were dried with magnesium sulphate and filtered. The filtrate was evaporated under reduced pressure and the residue was worked up by column chromatography (silica gel, ethyl acetate).

43 mg (31% of theory) 1-(phenylethynyl)-3-(4-trifluoromethylpyridin-3-yl)-imidazo[1,5-a]-pyridine were obtained.

¹H NMR (400 MHz, CDCl₃), 8.98 (d, 1H), 8.89 (s, 1H), 7.76 (m, 2H), 7.56 (m, 3H), 7.31 (m, 3H), 6.93 (dd, 1H), 6.64 (dd, 1H); ¹³C-NMR (100 MHz, CDCl₃), 153.4, 152.0, 138.8, 138.1, 133.9, 131.4, 131.1, 128.3, 128.1, 123.4, 122.8, 121.6, 121.3, 120.4, 118.7, 115.7, 114.4, 92.9, 82.1; MS (CI), m/z 364 (M⁺+1, 100), 264 (60).

The compounds of structure (I) in Table 1—especially of structure (IA) and (Ib)—are/were also prepared by analogy to the preparation examples 1 to 8 and in correspondence with the general description of the methods of preparation of the invention.

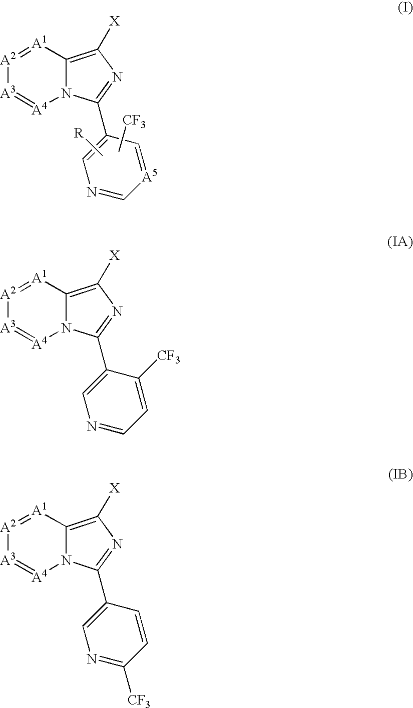

TABLE 1
Examples for compounds of structure (I)

| Ex. | | | | | | |
|---|---|---|---|---|---|---|
| no. | Structure | X | A¹ | A² | A³ | A⁴ |
| 9 | (IB) | 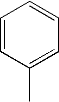 | CH | CH | CH | CH |
| 10 | (IA) | 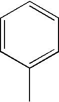 | CH | CH | CH | CH |
| 11 | (IB) | H | CH | CH | CH | CH |
| 12 | (IA) | C₃H₇-n | CH | CH | CH | CH |
| 13 | (IA) | CH₃ | CH | CH | CH | CH |
| 14 | (IB) | CH₃ | CH | CH | CH | CH |
| 15 | (IB) | H | N | C—OCH₃ | CH | C—OCH₃ |
| 16 | (IA) | CH₃ | C—Cl | CH | C—CF₃ | CH |
| 17 | (IA) | CH₃ | CH | Benzo- | — | CH |
| | | | | anellation at | | |
| | | | | A²-A³ | | |
| 18 | (IB) | CH₃ | CH | Benzo- | — | CH |
| | | | | anellation at | | |
| | | | | A²-A³ | | |
| 19 | (IA) | 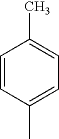 | CH | CH | CH | CH |
| 20 | (IA) | 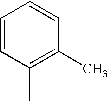 | CH | CH | CH | CH |
| 21 | (IA) | 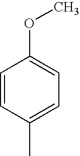 | CH | CH | CH | CH |
| 22 | (IA) | H | N | C—OCH₃ | CH | C—OCH₃ |
| 23 | (IA) | 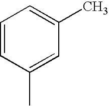 | C | CH | CH | CH |
| 24 | (IA) | 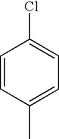 | CH | CH | CH | CH |
| 25 | (IA) | 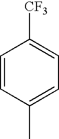 | CH | CH | CH | CH |
| 26 | (IA) | 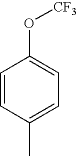 | CH | CH | CH | CH |
| 27 | (IA) | 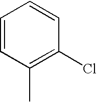 | CH | CH | CH | CH |
| 28 | (IA) | I (Iodo) | CH | CH | CH | CH |
| 29 | (IA) | H | C—Cl | CH | C—Cl | CH |
| 30 | (IA) | CH₃ | C—Cl | CH | C—Cl | CH |
| 31 | (IB) | CH₃ | C—Cl | CH | C—Cl | CH |
| 32 | (IA) | 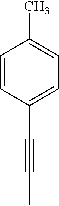 | CH | CH | CH | CH |
| 33 | (IA) | 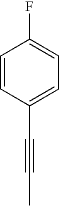 | CH | CH | CH | CH |
| 34 | (IA) | 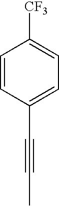 | CH | CH | CH | CH |
| 35 | (IA) | 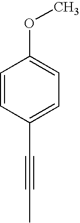 | CH | CH | CH | CH |
| 36 | (IA) | 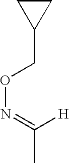 | CH | CH | CH | CH |
| 37 | (IA) | H | C—Cl | CH | C—CF₃ | CH |
| 38 | (IA) | Cl | C—Cl | CH | C—CF₃ | CH |
| 39 | (IA) | H | CH | N | C—CH₃ | CH |
| 40 | (IA) | 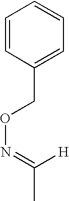 | CH | CH | CH | CH |
| 41 | (IA) | 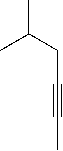 | CH | CH | CH | CH |
| 42 | (IA) | 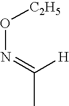 | CH | CH | CH | CH |
| 43 | (IA) | 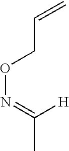 | CH | CH | CH | CH |
| 44 | (IA) | 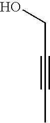 | CH | CH | CH | CH |
| 45 | (IA) | 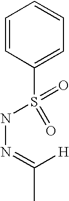 | CH | CH | CH | CH |
| 46 | (IA) | H | CH | CH | CH | C—CF₃ |
| 47 | (IA) | 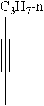 | CH | CH | CH | CH |
| 48 | (IA) | 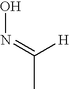 | CH | CH | CH | CH |
| 49 | (IA) | 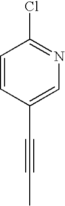 | CH | CH | CH | CH |
| 50 | (IA) | 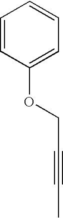 | CH | CH | CH | CH |
| 51 | (IA) | H | CH | CH | C—CF₃ | CH |
| 52 | (IA) | 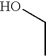 | CH | CH | CH | CH |
| 53 | (IA) | 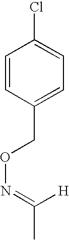 | CH | CH | CH | CH |
| 54 | (IA) | H | C—Cl | CH | CH | CH |
| 55 | (IA) | H | N | CH | CH | CH |
| 56 | (IA) | CF₂H | CH | CH | CH | CH |
| 57 | (IA) | 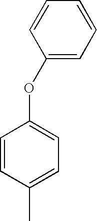 | CH | CH | CH | CH |
| 58 | (IA) | 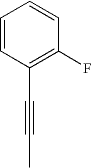 | CH | CH | CH | CH |
| 59 | (IA) | 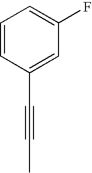 | CH | CH | CH | CH |
| 60 | (IA) | 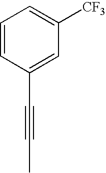 | CH | CH | CH | CH |
| 61 | (IA) | CH₂—O—CH₃ | CH | CH | CH | CH |
| 62 | (IA) | 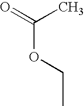 | CH | CH | CH | CH |
| 63 | (IA) | 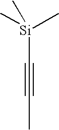 | CH | CH | CH | CH |
| 64 | (IA) | 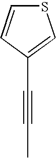 | CH | CH | CH | CH |
| 65 | (IA) | 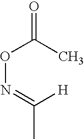 | CH | CH | CH | CH |
| 66 | (IA) | 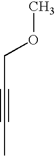 | CH | CH | CH | CH |
| 67 | (IA) | 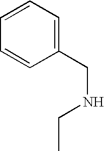 | CH | CH | CH | CH |
| 68 | (IA) | 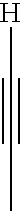 | CH | CH | CH | CH |
| 69 | (IA) | 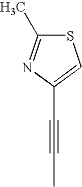 | CH | CH | CH | CH |
| 70 | (IA) | 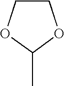 | CH | CH | CH | CH |
| 71 | (IA) | 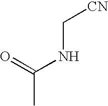 | CH | CH | CH | CH |
| 72 | (IA) | 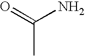 | CH | CH | CH | CH |
| 73 | (IA) | 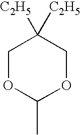 | CH | CH | CH | CH |
| 74 | (IA) | 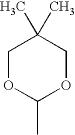 | CH | CH | CH | CH |
| 75 | (IA) | 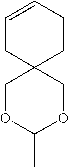 | CH | CH | CH | CH |
| 76 | (IA) | 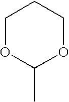 | CH | CH | CH | CH |
| 77 | (IA) | H | N | C—OCH₃ | CH | C—Cl |
| 78 | (IA) | C₄H₉-n | CH | CH | CH | CH |
| 79 | (IA) | 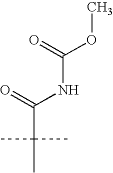 | CH | CH | CH | CH |
| 80 | (IA) | 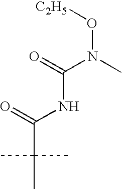 | CH | CH | CH | CH |
| 81 | (IA) | 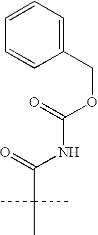 | CH | CH | CH | CH |
| 82 | (IA) | H | CH | CH | C—CH₃ | C—CH₃ |
| 83 | (IA) | H | CH | C—C(CH₃)₃ | CH | CH |
| 84 | (IA) | Br | N | CH | CH | CH |
| 85 | (IA) | H | N | C—CH₃ | CH | C—CH₃ |
| 86 | (IA) | 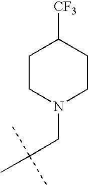 | CH | CH | CH | CH |
| 87 | (IA) | 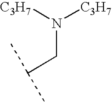 | CH | CH | CH | CH |
| 88 | (IA) | 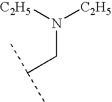 | CH | CH | CH | CH |
| 89 | (IA) | Br | N | C—OCH₃ | CH | C—Cl |
| 90 | (IA) | 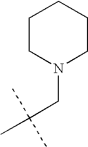 | CH | CH | CH | CH |
| 91 | (IA) | 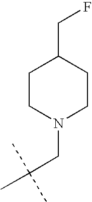 | CH | CH | CH | CH |
| 92 | (IA) | 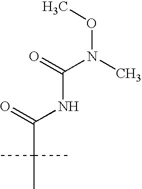 | CH | CH | CH | CH |
| 93 | (IA) | H | CH | CH | C—OCH₃ | CH |
| 94 | (IA) | H | C—F | CH | C—F | CH |
| 95 | (IA) | Cl | CH | CH | C—OCH₃ | C—Cl |
| 96 | (IA) | H | CH | CH | C—F | CH |
| 97 | (IA) | H | CH | C—F | CH | CH |
| 98 | (IA) | 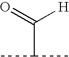 | CH | CH | C—OCH₃ | CH |
| 99 | (IA) | H | C—CH₃ | CH | C—CH₃ | CH |
| 100 | (IA) | H | C—CH₃ | CH | CH | CH |
| 101 | (IA) | Br | CH | CH | C—OCH₃ | C—Br |
| 102 | (IA) | H | N | C—Cl | CH | C—Cl |
| 103 | (IA) | Cl | CH | CH | C—F | CH |
| 104 | (IA) | Br | C—F | CH | C—F | CH |
| 105 | (IA) | Cl | C—CH₃ | CH | CH | CH |
| 106 | (IA) | 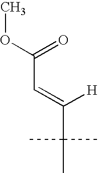 | CH | CH | CH | CH |
| 107 | (IA) | 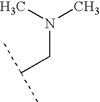 | CH | CH | CH | CH |
| 108 | (IA) | 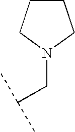 | CH | CH | CH | CH |
| 109 | (IA) | Cl | C—CH₃ | CH | C—CH₃ | CH |
| 110 | (IA) | Cl | C—CH₃ | CH | C—CH₃ | C—Cl |
| 111 | (IA) | Br | C—CH₃ | C—Br | C—CH₃ | C—Br |
| 112 | (IA) | Br | C—CH₃ | CH | CH | C—Br |
| 113 | (IA) | Cl | CH | CH | C—OCH₃ | C—Br |
| 114 | (IA) | Br | CH | CH | C—F | CH |
| 115 | (IA) | Cl | C—F | CH | C—F | CH |
| 116 | (IA) | 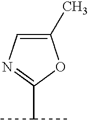 | CH | CH | CH | CH |
| 117 | (IA) | 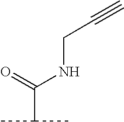 | CH | CH | CH | CH |
| 118 | (IA) | 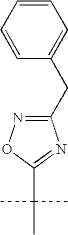 | CH | CH | CH | CH |
| 119 | (IA) | 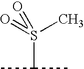 | CH | CH | CH | CH |
| 120 | (IA) | 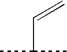 | CH | CH | CH | CH |
| 121 | (IA) | Br | C—CH₃ | CH | CH | CH |
| 122 | (IA) | 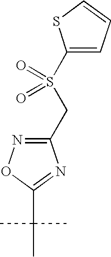 | CH | CH | CH | CH |
| 123 | (IA) | 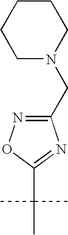 | CH | CH | CH | CH |
| 124 | (IA) | 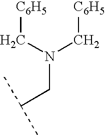 | CH | CH | CH | CH |
| 125 | (IA) | 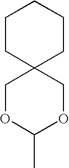 | CH | CH | CH | CH |
| 126 | (IA) | COOH | CH | CH | CH | CH |
| 127 | (IA) | 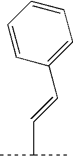 | CH | CH | CH | CH |
| 128 | (IA) | SCH₃ | CH | CH | CH | CH |
| 129 | (IA) | 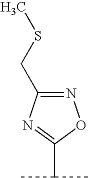 | CH | CH | CH | CH |

Further physical data for the compounds in Table 1:

### Ex. No. 9

¹H NMR (400 MHz, CDCl₃), 9.25 (s, 1H), 8.39 (dd, 1H), 8.27 (d, 1H), 7.92 (d, 3H), 7.84 (d, 1H), 7.48 (m, 2H), 7.35 (m, 1H), 6.90 (dd, 1H), 6.75 (dd, 1H).

### Ex. No. 10

¹H NMR (400 MHz, CDCl₃), 8.96 (s, 1H), 8.92 (s, 1H), 7.73 (d, 1H), 7.58 (m, 2H), 7.55-7.46 (m, 4H), 7.36 (m, 1H), 6.78 (dd, 1H), 6.66 (dd, 10H).

### Ex. No. 11

¹H NMR (400 MHz, CDCl₃), 9.19 (d, 1H), 8.32 (dd, 1H), 8.22 (d, 1H), 7.79 (d, 1H), 7.48 (d, 1H), 7.40 (s, 1H), 6.73 (dd, 1H), 6.62 (dd, 1H).

### Ex. No. 12

¹H NMR (400 MHz, CDCl₃), 8.94 (d, 1H), 8.86 (s, 1H), 7.75 (d, 1H), 7.52 (d, 1H), 7.45 (d, 1H), 6.68 (dd, 1H), 6.46 (dd, 1H), 2.90 (t, 2H), 1.79 (sext, 2H), 0.97 (t, 3H).

### Ex. No. 13

¹H NMR (400 MHz, CDCl₃), 8.93 (d, 1H), 8.85 (s, 1H), 7.76 (d, 1H), 7.49 (d, 1H), 7.43 (d, 1H), 6.67 (dd, 1H), 6.49 (dd, 10H), 2.59 (s, 3H).

### Ex. No. 14

¹H NMR (400 MHz, CDCl₃), 9.18 (d, 1H), 8.34 (dd, 1H), 8.21 (d, 1H), 7.82 (d, 1H), 7.46 (d, 1H), 6.75 (dd, 1H), 6.64 (dd, 1H), 2.59 (s, 3H).

### Ex. No. 15

¹H NMR (400 MHz, CDCl₃), 8.92 (d, 1H), 8.15 (dd, 1H), 7.74 (d, 1H), 7.41 (s, 1H), 5.58 (s, 1H), 4.02 (s, 3H), 3.97 (s, 3H).

### Ex. No. 16

¹H NMR (400 MHz, CDCl₃), δ 9.04 (d, 1H), 8.84 (s, 1H), 7.78 (d, 1H), 7.66 (s, 1H), 6.82 (s, 1H), 2.85 (s, 3H).

### Ex. No. 17

¹H NMR (400 MHz, CDCl₃), 8.98 (d, 1H), 8.87 (s, 1H), 8.16 (d, 1H), 7.75 (d, 1H), 7.60 (m, 2H), 7.44 (t, 1H), 7.27 (d, 1H), 6.78 (d, 1H), 2.88 (s, 3H). MS (CI), m/z 342 (M⁺+2+Na, 10), 328 (M⁺+1, 100).

### Ex. No. 18

¹H NMR 400 MHz, CDCl₃), 9.16 (d, 1H), 8.27 (dd, 1H), 8.14 (d, 1H), 7.92 (d, 1H), 7.80 (d, 1H), 7.57 (m, 2H), 7.42 (m, 1H), 6.84 (d, 1H), 2.86 (s, 3H).

### Ex. No. 19

¹H NMR (400 MHz, CDCl₃), δ 8.96 (d, 1H), 8.94 (s, 1H), 8.09 (d, 1H), 7.80 (d, 2H), 7.77 (d, 1H), 7.57 (d, 1H), 7.27 (d, 2H), 6.83 (dd, 1H), 6.58 (dd, 1H), 2.41 (s, 3H).

### Ex. No. 20

¹H NMR (400 MHz, CDCl₃), 8.97 (s, 1H), 8.95 (d, 1H), 7.78 (d, 1H), 7.63 (d, 1H), 7.47 (m, 2H), 7.23-7.35 (m, 3H), 6.78 (dd, 1H), 6.59 (dd, 1H), 2.42 (s, 3H).

### Ex. No. 21

¹H NMR (400 MHz, CDCl₃), 8.96 (d, 1H), 8.94 (s, 1H), 7.83 (m, 3H), 7.78 (d, 1H), 7.58 (d, 1H), 7.02 (d, 2H), 6.81 (dd, 1H), 6.56 (dd, 1H), 3.86 (s, 3H).

### Ex. No. 23

¹H-NMR (400 MHz, CDCl₃), 8.98 (d, 1H), 8.94 (s, 1H), 7.92 (d, 1H), 7.78 (s, 1H), 7.76 (d, 10H), 7.69 (d, 1H), 7.57 (d, 1H), 7.37 (dd, 1H), 7.14 (d, 1H), 6.84 (dd, 1H), 6.58 (dd, 1H), 2.41 (s, 3H).

### Ex. No. 24

¹H NMR (400 MHz, CDCl₃), 8.97 (d, 1H), 8.93 (s, 1H), 7.83 (m, 3H), 7.78 (d, 1H), 7.58 (d, 1H), 7.42 (d, 2H), 6.88 (dd, 1H), 6.60 (dd, 1H).

### Ex. No. 25

¹H NMR (400 MHz, CDCl₃), 8.99 (d, 1H), 8.95 (s, 1H), 8.04 (d, 2H), 7.92 (d, 1H), 7.78 (d, 1H), 7.71 (d, 2H), 7.63 (d, 1H), 6.95 (dd, 1H), 6.63 (dd, 1H).

### Ex. No. 26

¹H NMR (400 MHz, CDCl₃), 8.98 (d, 1H), 8.94 (s, 1H), 7.94 (d, 2H), 7.86 (d, 1H), 7.78 (d, 1H), 7.60 (d, 1H), 7.33 (d, 2H), 6.92 (dd, 1H), 6.61 (dd, 1H).

### Ex. No. 27

¹H NMR (400 MHz, CDCl₃), 8.98 (d, 1H), 8.97 (s, 1H), 7.78 (d, 1H), 7.52-7.67 (m, 4H), 7.33 (m, 2H), 6.88 (dd, 1H), 6.64 (dd, 1H).

### Ex. No. 28

¹H NMR (400 MHz, CDCl₃) 8.97 (d, 1H), 8.87 (s, 1H), 7.76 (d, 1H), 7.55 (d, 1H), 7.42 (d, 1H), 6.87 (dd, 1H), 6.61 (dd, 1H); MS (EI), m/z 389 (M⁺, 100).

### Ex. No. 29

¹H NMR (400 MHz, CDCl₃), 8.98 (d, 1H), 8.85 (s, 1H), 7.81 (s, 1H), 7.76 (d, 1H), 7.57 (s, 1H), 6.86 (s, 1H). MS (CI), m/z 334 (M⁺+2, 55), 332 (M⁺, 100), 300 (20), 298 (60).

### Ex. No. 30

¹H NMR (400 MHz, CDCl₃), 9.00 (d, 1H), 8.83 (s, 1H), 7.77 (d, 1H), 7.40 (s, 1H), 6.74 (s, 1H), 2.84 (s, 3H). MS (CI), m/z 348 (M⁺+2, 60), 346 (M⁺, 100), 314 (20), 298 (50).

### Ex. No. 31

¹H NMR (400 MHz, CDCl₃),), 9.15 (d, 10H), 8.26 (dd, 10H), 8.12 (s, 1H), 7.84 (d, 1H), 6.78 (s, 1H), 2.82 (s, 3H). MS (CI), m/z 346 (M⁺, 100).

### Ex. No. 32

¹H NMR (400 MHz, CDCl₃), 8.98 (d, 1H), 8.91 (s, 1H), 7.75 (m, 2H), 7.57 (d, 1H), 7.48 (d, 2H), 7.16 (d, 2H), 6.94 (dd, 1H), 6.63 (dd, 1H), 2.38 (s, 3H); ¹³C-NMR (100 MHz, CDCl₃), MS: 153.4, 151.9, 138.8, 138.5, 138.2, 133.9, 131.4, 131.0, 129.1, 123.6, 121.6, 121.2, 120.9, 120.3, 118.7, 115.9, 114.4, 93.0, 81.4, 21.5; MS (CI), m/z 364 (M⁺+1, 100).

### Ex. No. 33

¹H NMR (400 MHz, CDCl₃), 8.97 (d, 1H), 8.88 (s, 1H), 7.76 (m, 2H), 7.55 (m, 3H), 7.05 (d, 2H), 6.96 (dd, 1H), 6.65 (dd, 1H); MS (CI), m/z 382 (M+1, 100).

### Ex. No. 34

¹H NMR (400 MHz, CDCl₃), 8.99 (d, 1H), 8.88 (s, 1H), 7.77 (m, 2H), 7.66 (m, 2H), 7.60 (m, 3H), 6.99 (dd, 1H), 6.68 (dd, 1H); MS (CI), m/z 432 (M⁺+1, 66), 264 (100).

### Ex. No. 35

¹H NMR (400 MHz, CDCl₃), 8.98 (d, 1H), 8.90 (s, 1H), 7.76 (m, 2H), 7.54 (m, 3H), 6.85-6.96 (m, 3H), 6.62 (dd, 1H), 3.83 (s, 3H).

### Ex. No. 36

¹H NMR (400 MHz, CDCl₃), 8.97 (d, 1H), 8.86 (s, 1H), 8.47 (s, 1H), 8.05 (d, 1H), 7.76 (d, 1H), 7.55 (d, 1H), 6.97 (dd, 1H), 6.68 (dd, 1H), 4.04 (d, 2H), 1.26 (m, 1H), 0.61 (m, 2H), 0.37 (m, 2H);

MS (CI), m/z 361 (M⁺+1, 100), 289 (92).

### Ex. No. 37

¹H NMR (400 MHz, CD₃CN), 9.03 (d, 1H), 8.86 (s, 1H), 8.18 (s, 1H), 7.88 (d, 1H), 7.82 (s, 1H), 7.14 (s, 1H).

### Ex. No. 38

¹H NMR (400 MHz, CD₃CN), 9.04 (d, 1H), 8.86 (s, 1H), 8.11 (s, 1H), 7.88 (d, 1H), 7.14 (s, 1H).

### Ex. No. 39

¹H NMR (400 MHz, CDCl₃) 9.18 (s, 1H), 9.05 (d, 1H), 8.87 (s, 1H), 8.11 (s, 1H), 7.82 (d, 1H), 7.37 (s, 1H), 2.43 (s, 3H)

### Ex. No. 40

¹H NMR (400 MHz, CDCl₃) 8.98 (d, 1H), 8.85 (s, 1H), 8.52 (s, 1H), 8.04 (d, 1H), 7.76 (d, 1H), 7.58 (d, 1H), 7.47 (d, 2H), 7.32-7.43 (m, 3H), 6.98 (dd, 1H), 6.65 (dd, 1H), 5.24 (s, 2H)

### Ex. No. 41

¹H NMR (400 MHz, CDCl₃) 8.96 (d, 1H), 8.84 (s, 1H), 7.78 (d, 1H), 7.64 (d, 1H), 7.52 (d, 1H), 6.86 (dd, 1H), 6.59 (dd, 1H), 2.42 (d, 2H), 1.95 (sept, 1H), 1.12 (d, 6H)

### Ex. No. 42

¹H NMR (400 MHz, CDCl₃) 8.97 (d, 1H), 8.86 (s, 1H), 8.54 (s, 1H), 8.07 (d, 1H), 7.78 (d, 1H), 7.58 (d, 1H), 6.97 (dd, 1H), 6.67 (dd, 1H), 4.22 (q, 2H), 1.36 (t, 3H)

### Ex. No. 43

¹H NMR (400 MHz, CDCl₃) 8.98 (d, 1H), 8.84 (s, 1H), 8.49 (s, 1H), 8.05 (d, 1H), 7.76 (d, 1H), 7.59 (d, 1H), 6.98 (dd, 1H), 6.69 (dd, 1H), 6.06 (m, 1H), 5.38 (d, 1H), 5.25 (d, 1H), 4.71 (d, 2H)

### Ex. No. 44

¹H NMR (400 MHz, CDCl₃) 8.97 (d, 1H), 8.88 (s, 1H), 7.78 (d, 1H), 7.64 (s, 2H), 7.61 (d, 1H), 7.56 (d, 1H), 6.79 (dd, 1H), 6.57 (dd, 1H)

### Ex. No. 45

¹H NMR (400 MHz, CDCl₃) 12.68 (s, 1H) 9.03 (d, 1H), 8.90 (s, 1H), 7.96 (d, 2H), 7.82 (d, 1H), 7.76 (d, 1H), 7.70 (d, 1H), 7.60 (s, 1H), 7.44 (m, 3H), 7.09 (dd, 1H), 6.81 (dd, 1H)

### Ex. No. 46

¹H NMR (400 MHz, CDCl₃) 8.95 (d, 1H), 8.80 (s, 1H), 7.85 (s, 1H), 7.78 (d, 1H), 7.64 (d, 1H), 7.19 (d, 1H), 6.82 (dd, 1H)

### Ex. No. 47

¹H NMR (400 MHz, CDCl₃) 8.96 (d, 1H), 8.84 (s, 1H), 7.76 (d, 1H), 7.62 (d, 1H), 7.52 (d, 1H), 6.84 (dd, 1H), 6.58 (dd, 1H), 2.48 (t, 2H), 1.63 (sext, 2H), 1.08 (t, 3H)

### Ex. No. 48

¹H NMR (400 MHz, CDCl₃) 8.98 (d, 1H), 8.88 (s, 1H), 8.52 (s, 1H), 8.03 (d, 1H), 7.78 (d, 1H), 7.59 (d, 1H), 7.06 (br. s, 1H), 6.99 (dd, 1H), 6.72 (dd, 1H)

### Ex. No. 49

¹H NMR (400 MHz, CDCl₃) 8.99 (d, 1H), 8.92 (s, 1H), 8.60 (d, 1H), 7.76-7.85 (m, 3H), 7.60 (d, 1H), 7.35 (d, 1H), 7.02 (dd, 1H), 6.74 (dd, 1H)

### Ex. No. 50

¹H NMR (400 MHz, CDCl₃) 8.98 (d, 1H), 8.84 (s, 1H), 7.76 (d, 1H), 7.60 (d, 1′), 7.54 (d, 1H), 7.32 (m, 2H), 7.05 (d, 2H), 6.98 (dd, 1H), 6.92 (m, 1H), 6.62 (dd, 1H), 5.02 (s, 2H)

### Ex. No. 51

¹H NMR (400 MHz, CDCl₃) 9.02 (d, 1H), 8.89 (s, 10H), 7.92 (s, 1H), 7.80 (d, 1H), 7.76 (s, 1H), 7.63 (d, 1H), 6.92 (d, 1H)

### Ex. No. 52

¹H NMR (400 MHz, CDCl₃) 8.96 (d, 1H), 8.85 (s, 1H), 7.76 (d, 1H), 7.62 (d, 1H), 7.56 (d, 1H), 6.90 (dd, 1H), 6.57 (dd, 1H), 5.02 (s, 2H), 2.38 (br. s, 1H)

### Ex. No. 53

¹H NMR (400 MHz, CDCl₃) 8.97 (d, 1H), 8.86 (s, 1H), 8.51 (s, 1H), 7.98 (d, 1H), 7.77 (d, 1H), 7.58 (d, 1H), 7.40 (d, 2H), 7.35 (d, 2H), 6.98 (dd, 1H), 6.68 (dd, 1H), 5.20 (s, 2H)

### Ex. No. 54

¹H NMR (400 MHz, CDCl₃) 8.96 (d, 1H), 8.86 (s, 1H), 7.78 (m, 2H), 7.54 (d, 1H, 6.85 (d, 1H), 6.54 (dd, 1H)

### Ex. No. 55

¹H NMR (400 MHz, CDCl₃) 8.98 (d, 1H), 8.88 (s, 1H), 8.26 (m, 1H), 7.88 (m, 2H), 7.78 (d, 1H), 6.60 (dd, 10H)

### Ex. No. 56

¹H NMR (400 MHz, CDCl₃) 8.99 (d, 1H), 8.86 (s, 1H), 7.80 (d, 1H), 7.76 (d, 1H), 7.59 (d, 1H), 6.82-7.24 (t, 1H), 6.96 (dd, 1H), 6.68 (dd, 1H)

### Ex. No. 57

¹H NMR (400 MHz, CDCl₃) 8.97 (d, 1H), 8.96 (s, 10H), 7.91-7.85 (m, 3H), 7.78 (d, 1H), 7.59 (d, 10H), 7.39-7.33 (m, 2H), 7.17-7.05 (m, 5H), 6.85 (dd, 10H), 6.59 (dd, 10H).

### Ex. No. 58

¹H NMR (400 MHz, CDCl₃) 9.00 (d, 1H), 8.91 (s, 1H), 7.81-7.76 (m, 2H), 7.62-7.57 (m, 2H), 7.35-7.26 (m, 1H), 7.17-7.09 (m, 2H), 6.98 (dd, 1H), 6.67 (dd, 1H).

### Ex. No. 59

¹H NMR (400 MHz, CDCl₃) 9.00 (d, 10H), 8.88 (s, 1H), 7.80-7.75 (m, 2H), 7.58 (d, 1H), 7.39-7.24 (m, 3H), 7.06-6.97 (m, 2H), 6.68 (dd, 1H).

### Ex. No. 60

¹H NMR (400 MHz, CDCl₃) 9.02 (d, 1H), 8.92 (s, 1H), 7.85-7.76 (m, 4H), 7.62-7.55 (m, 2H), 7.46 (dd, 1H), 7.02 (dd, 1H), 6.70 (dd, 1H).

### Ex. No. 61

¹H NMR (400 MHz, CDCl₃) 8.96 (d, 1H), 8.87 (s, 1H), 7.75 (d, 1H), 7.64 (d, 1H), 7.56 (d, 1H), 6.82 (dd, 1H), 6.58 (dd, 1H), 4.83 (s, 2H), 3.44 (s, 3H).

### Ex. No. 62

¹H NMR (400 MHz, CDCl₃) 8.97 (d, 1H), 8.88 (s, 1H), 7.78 (d, 1H), 7.71 (d, 1H), 7.56 (d, 10H), 6.88 (dd, 10H), 6.60 (dd, 1H), 5.44 (s, 2H), 2.12 (s, 3H).

### Ex. No. 63

¹H NMR (400 MHz, CDCl₃) 8.98 (d, 1H), 8.84 (s, 1H), 7.74 (d, 1H), 7.66 (d, 1H), 7.55 (d, 1H), 6.94 (dd, 1H), 6.63 (dd, 1H), 0.28 (s, 9H).

### Ex. No. 64

¹H NMR (400 MHz, CDCl₃) 8.98 (d, 1H), 8.89 (s, 1H), 7.78 (d, 1H), 7.74 (d, 1H), 7.60-7.55 (m, 2H), 7.30 (m, 1H), 7.24 (m, 1H), 6.96 (dd, 1H), 6.64 (dd, 1H).

### Ex. No. 65

¹H NMR (400 MHz, CDCl₃) 9.00 (d, 1H), 8.90 (s, 1H), 8.68 (s, 1H), 8.30 (d, 1H), 7.79 (d, 1H), 7.64 (d, 1H), 7.15 (dd, 1H), 6.79 (dd, 1H), 2.25 (s, 3H).

### Ex. No. 66

¹H NMR (400 MHz, CDCl₃) 8.98 (d, 1H), 8.86 (s, 1H), 7.76 (d, 1H), 7.67 (d, 10H), 7.57 (d, 1H), 6.95 (dd, 1H), 6.64 (dd, 1H), 4.44 (s, 2H), 3.51 (s, 3H).

### Ex. No. 67

¹H NMR (400 MHz, CDCl₃) 8.96 (d, 1H), 8.87 (s, 1H), 7.76 (d, 1H), 7.56 (d, 1H), 7.40-7.22 (m, 6H), 6.75 (dd, 1H), 6.54 (dd, 1H), 4.18 (s, 2H), 3.86 (s, 2H), 3.02 (br. s, NH).

### Ex. No. 68

¹H NMR (400 MHz, CDCl₃) 8.98 (d, 1H), 8.86 (s, 1H), 7.77 (d, 1H), 7.68 (d, 1H), 7.58 (d, 1H), 6.96 (dd, 1H), 6.66 (dd, 1H), 3.42 (s, 1H).

### Ex. No. 69

¹H NMR (400 MHz, CDCl₃) 8.98 (d, 1H), 8.89 (s, 1H), 7.80 (d, 1H), 7.77 (d, 1H), 7.59 (d, 1H), 7.42 (s, 1H), 6.96 (dd, 1H), 6.67 (dd, 1H), 2.77 (s, 3H).

### Ex. No. 70

¹H NMR (400 MHz, CDCl₃) 8.96 (d, 1H), 8.88 (s, 1H), 7.78-7.74 (m, 2H), 7.56 (d, 1H), 6.84 (dd, 1H), 6.59 (dd, 10H), 6.26 (s, 1H), 4.25 (m, 2H), 4.11 (m, 2H).

### Ex. No. 71

¹H NMR (400 MHz, CDCl₃) 9.04 (d, 1H), 8.88 (s, 1H), 8.38 (d, 1H), 7.80 (d, 1H), 7.65 (d, 1H), 7.43 (t, NH), 7.18 (dd, 1H), 6.79 (dd, 1H), 4.21 (s, 2H).

### Ex. No. 72

¹H NMR (400 MHz, CDCl₃) 9.02 (d, 1H), 8.90 (s, 1H), 8.40 (d, 1H), 7.79 (d, 1H), 7.64 (d, 1H), 7.15 (dd, 1H), 7.02 (br. s, 2 NH), 6.77 (dd, 1H).

### Ex. No. 73

¹H NMR (400 MHz, CDCl₃) 8.95 (d, 1H), 8.83 (s, 10H), 7.92 (d, 1H), 7.75 (d, 1H), 7.52 (d, 1H), 6.84 (dd, 1H), 6.58 (dd, 1H), 5.88 (s, 1H), 4.01 (d, 2H), 3.66 (d, 2H), 1.92 (q, 1H), 1.32 (q, 2H), 1.16 (q, 1H), 0.95 (t, 1H), 0.86 (t, 2H), 0.82 (t, 3H).

### Ex. No. 74

¹H NMR (400 MHz, CDCl₃) 8.94 (d, 1H), 8.84 (s, 1H), 7.95 (d, 1H), 7.73 (d, 1H), 7.52 (d, 1H), 6.83 (dd, 10H), 6.58 (dd, 1H), 5.87 (s, 1H), 3.82 (d, 2H), 3.74 (d, 2H), 1.39 (s, 3H), 0.84 (s, 3H).

### Ex. No. 75

¹H NMR (400 MHz, CDCl₃) 8.95 (d, 1H), 8.84 (s, 1H), 7.92 (d, 1H), 7.74 (d, 1H), 7.51 (d, 1H), 6.84 (dd, 1H), 6.58 (dd, 1H), 5.93 (s, 1H), 5.74-5.65 (m, 1H), 5.62-5.57 (m, 1H), 4.06 (dd, 2H), 3.73 (m, 2H), 2.52 (m, 1H), 2.36 (m, 1H), 2.22 (m, 1H), 2.04 (m, 1H), 1.81 (m, 1H), 1.68 (m, 1H).

### Ex. No. 76

¹H NMR (400 MHz, CDCl₃) 8.86 (d, 1H), 8.78 (s, 1H), 7.84 (d, 1H), 7.65 (d, 1H), 7.42 (d, 1H), 6.76 (dd, 1H), 6.50 (dd, 1H), 5.94 (s, 1H), 4.29-4.22 (m, 2H), 4.04-3.97 (m, 2H), 2.35-2.20 (m, 1H), 1.50-1.42 (m, 1H).

### Ex. No. 77

¹H-NMR (400 MHz, CDCl₃) 8.96 (d, 1H), 8.83 (s, 1H), 7.69 (d, 1H), 7.49 (s, 1H), 6.38 (s, 1H), 4.04 (s, 3H).

### Ex. No. 78

¹H NMR (400 MHz, CDCl₃) 8.97 (d, 1H), 8.89 (s, 1H), 7.76 (d, 1H), 7.62 (d, 1H), 7.54 (d, 1H), 6.78 (dd, 1H), 6.56 (dd, 1H), 2.03 (m, 1H), 1.82 (m, 1H), 1.50-1.35 (m, 4H), 0.94 (t, 3H).

### Ex. No. 79

¹H NMR (400 MHz, CDCl₃), 9.19 (s, NH), 8.96 (d, 1H), 8.82 (s, 1H), 8.35 (d, 1H), 7.74 (d, 1H), 7.63 (d, 1H), 7.17 (dd, 1H), 6.79 (dd, 1H), 3.79 (s, 3H).

### Ex. No. 80

¹H NMR (400 MHz, CDCl₃) 10.42 (s, NH), 9.09 (d, 1H), 8.99 (s, 1H), 8.55 (d, 1H), 7.88 (d, 1H), 7.83 (d, 1H), 7.25 (dd, 1H), 6.92 (dd, 1H), 4.04 (q, 2H), 3.32 (s, 3H), 1.37 (t, 3H).

### Ex. No. 81

¹H NMR (400 MHz, CDCl₃) 9.23 (s, NH), 8.97 (d, 1H), 8.80 (s, 1H), 8.38 (d, 1H), 7.74 (d, 10H), 7.60 (d, 1H), 7.38-7.25 (m, 5H), 7.18 (dd, 1H), 6.78 (dd, 1H), 5.21 (d, 2H).

### Ex. No. 82

¹H NMR (400 MHz, CDCl₃) 8.84 (d, 1H), 8.79 (s, 1H), 7.59 (d, 1H), 7.52 (s, 1H), 7.32 (d, 1H), 6.61 (d, 1H), 2.16 (s, 3H), 1.92 (s, 3H).

### Ex. No. 83

¹H NMR (400 MHz, CDCl₃) 8.94 (d, 1H), 8.77 (s, 1H), 7.75 (d, 1H), 7.59 (d, 1H), 7.57 (s, 1H), 7.39 (s, 1H), 6.65 (dd, 1H), 1.35 (s, 9H).

### Ex. No. 84

¹H NMR (400 MHz, CDCl₃) 8.97 (d, 1H), 8.80 (s, 10H), 8.18 (d, 1H), 7.91 (d, 1H), 7.74 (d, 10H), 7.58 (dd, 1H).

### Ex. No. 85

¹H NMR (400 MHz, CDCl₃) 8.90 (d, 1H), 8.81 (s, 1H), 7.63 (d, 1H), 7.61 (s, 1H), 6.24 (d, 1H), 2.44 (s, 3H), 1.96 (s, 3H).

### Ex. No. 86

¹H NMR (400 MHz, CDCl₃) 8.93 (d, 1H), 8.86 (s, 1H), 7.75 (d, 1H), 7.62 (d, 1H), 7.55 (d, 1H), 6.77 (dd, 1H), 6.56 (dd, 1H), 3.92 (s, 2H), 3.14-3.07 (m, 2H), 2.15-2.06 (m, 2H), 2.05-1.94 (m, 1H), 1.86-1.80 (m, 2H), 1.77-1.60 (m, 2H).

### Ex. No. 87

¹H NMR (400 MHz, CDCl₃) 8.92 (d, 1H), 8.86 (s, 1H), 7.75 (d, 1H), 7.68 (d, 1H), 7.54 (d, 1H), 6.73 (dd, 1H), 6.54 (dd, 1H), 3.98 (s, 2H), 2.49-2.42 (m, 4H), 1.62-1.52 (m, 4H), 0.86 (t, 6H).

### Ex. No. 88

¹H NMR (400 MHz, CDCl₃) 8.94 (d, 1H), 8.87 (s, 1H), 7.74 (d, 1H), 7.67 (d, 1H), 7.56 (d, 1H), 6.75 (dd, 1H), 6.53 (dd, 1H), 4.00 (s, 2H), 2.62 (q, 4H), 1.14 (t, 6H).

### Ex. No. 89

¹H NMR (400 MHz, CDCl₃) 8.95 (d, 1H), 8.84 (s, 1H), 7.75 (d, 1H), 6.37 (s, 1H), 4.08 (s, 3H).

### Ex. No. 90

¹H NMR (400 MHz, CDCl₃) 8.94 (d, 1H), 8.87 (s, 1H), 7.74 (d, 1H), 7.66 (d, 1H), 7.53 (d, 1H), 6.74 (dd, 1H), 6.52 (dd, 1H), 3.88 (s, 2H), 2.56-2.48 (m, 4H), 1.68-1.59 (m, 4H), 1.46-1.38 (m, 2H).

### Ex. No. 91

¹H NMR (400 MHz, CDCl₃) 8.94 (d, 1H), 8.87 (s, 1H), 7.76 (d, 1H), 7.64 (d, 1H), 7.55 (d, 1H), 6.76 (dd, 1H), 6.54 (dd, 1H), 4.33 (d, 1H), 4.20 (d, 1H), 3.94 (s, 2H), 3.09-3.02 (m, 2H), 2.17-2.09 (m, 2H), 1.75-1.64 (m, 3H), 1.45-1.37 (m, 2H).

### Ex. No. 92

¹H NMR (400 MHz, CDCl₃) 10.38 (s, NH), 9.07 (d, 1H), 8.97 (s, 1H), 8.52 (d, 1H), 7.89 (d, 1H), 7.84 (d, 1H), 7.24 (dd, 1H), 6.90 (dd, 1H), 3.97 (s, 3H), 3.31 (s, 3H).

### Ex. No. 93

¹H NMR (400 MHz, CDCl₃) 8.95 (d, 1H), 8.90 (s, 1H), 7.77 (d, 1H), 7.59 (s, 1H), 7.42 (d, 1H), 7.05 (s, 1H), 6.63 (dd, 1H), 3.68 (s, 3H).

### Ex. No. 94

¹H NMR (400 MHz, CDCl₃) 9.00 (d, 1H), 8.85 (s, 1H), 7.83 (s, 1H), 7.78 (d, 1H), 7.38 (d, 1H), 6.54 (dd, 1H).

### Ex. No. 95

¹H NMR (400 MHz, CDCl₃) 8.94 (d, 1H), 8.84 (s, 1H), 7.66 (d, 1H), 7.52 (d, 1H), 6.92 (d, 1H), 3.94 (s, 3H).

### Ex. No. 96

¹H NMR (400 MHz, CDCl₃) 8.99 (d, 1H), 8.87 (s, 1H), 7.78 (m, 2H), 7.42 (d, 1H), 6.76-6.46 (m, 2H).

### Ex. No. 97

¹H NMR (400 MHz, CDCl₃) 8.97 (d, 1H), 8.86 (s, 1H), 7.78 (d, 1H), 7.70 (s, 1H), 7.56-7.50 (m, 2H), 6.76 (dd, 1H).

### Ex. No. 98

¹H NMR (400 MHz, CDCl₃) 10.09 (s, 1H), 9.04 (d, 10H), 8.92 (s, 1H), 8.28 (d, 1H), 7.81 (d, 1H), 7.14 (d, 1H), 7.09 (m, 1H), 3.74 (s, 3H).

### Ex. No. 99

¹H NMR (400 MHz, CDCl₃) 8.94 (d, 1H), 8.86 (s, 1H), 7.76 (d, 1H), 7.57 (s, 1H), 7.27 (s, 1H), 6.45 (s, 1H), 2.56 (s, 3H), 2.16 (s, 3H).

### Ex. No. 100

¹H NMR (400 MHz, CDCl₃) 8.95 (d, 1H), 8.87 (s, 1H), 7.77 (d, 1H), 7.64 (s, 1H), 7.48 (d, 1H), 6.61 (d, 1H), 6.54 (dd, 1H), 2.49 (s, 3H).

### Ex. No. 101

hu 1H NMR (400 MHz, CDCl₃) 8.94 (d, 1H), 8.84 (s, 1H), 7.64 (d, 1H), 7.52 (d, 1H), 6.88 (d, 1H), 3.92 (s, 3H).

### Ex. No. 102

¹H NMR (400 MHz, CDCl₃) 8.97 (d, 1H), 8.86 (s, 1H), 7.82 (s, 1H), 7.72 (d, 1H), 6.74 (s, 1H).

### Ex. No. 103

¹H NMR (400 MHz, CDCl₃) 8.98 (d, 1H), 8.85 (s, 1H), 7.77 (d, 1H), 7.34 (d, 1H), 6.57-6.46 (m, 2H).

### Ex. No. 104

¹H NMR (400 MHz, CDCl₃) 9.00 (d, 1H), 8.92 (s, 1H), 7.78 (d, 1H), 7.36 (d, 1H), 6.55 (dd, 1H).

### Ex. No. 105

¹H NMR (400 MHz, CDCl₃) 8.96 (d, 1H), 8.84 (s, 1H), 7.76 (d, 1H), 7.37 (d, 1H), 6.54 (d, 1H), 6.48 (dd, 1H), 2.76 (s, 3H).

### Ex. No. 106

¹H NMR (400 MHz, CDCl₃) 8.98 (d, 1H), 8.89 (s, 1H), 7.96 (d, 1H), 7.78 (d, 1H), 7.74 (d, 1H), 7.58 (d, 1H), 7.02 (dd, 1H), 6.72 (d, 1H), 6.68 (dd, 1H), 3.82 (s, 3H).

### Ex. No. 107

¹H NMR (400 MHz, CDCl₃) 8.94 (d, 1H), 8.86 (s, 1H), 7.74 (d, 1H), 7.62 (d, 1H), 7.53 (d, 1H), 6.77 (dd, 1H), 6.56 (dd, 1H), 3.83 (s, 2H), 2.34 (s, 6H).

### Ex. No. 108

¹H NMR (400 MHz, CDCl₃) 8.93 (d, 1H), 8.87 (s, 1H), 7.74 (d, 1H), 7.63 (d, 1H), 7.54 (d, 1H), 6.75 (dd, 1H), 6.54 (dd, 1H), 4.02 (s, 2H), 2.66-2.60 (m, 4H), 1.82-1.77 (m, 4H).

### Ex. No. 109

¹H NMR (400 MHz, CDCl₃) 8.96 (d, 1H), 8.84 (s, 1H), 7.76 (d, 1H), 7.15 (s, 1H), 6.42 (s, 1H), 2.66 (s, 3H), 2.14 (s, 3H).

### Ex. No. 110

¹H NMR (400 MHz, CDCl₃) 8.97 (d, 1H), 8.85 (s, 1H), 7.77 (d, 1H), 7.24 (s, 1H), 6.77 (s, 1H), 2.67 (s, 3H), 2.13 (s, 3H).

### Ex. No. 111

¹H NMR (400 MHz, CDCl₃) 8.93 (d, 1H), 8.80 (s, 1H), 7.64 (d, 1H), 2.94 (s, 3H), 2.52 (s, 3H).

### Ex. No. 112

¹H NMR (400 MHz, CDCl₃) 8.98 (d, 1H), 8.82 (s, 1H), 7.76 (d, 1H), 7.23 (d, 1H), 6.68 (d, 1H), 2.85 (s, 3H).

### Ex. No. 113

¹H NMR (400 MHz, CDCl₃) 8.92 (d, 1H), 8.78 (s, 1H), 8.07 (d, 1H), 7.65 (d, 1H), 7.28 (d, 1H), 4.02 (s, 3H).

### Ex. No. 114

¹H NMR (400 MHz, CDCl₃) 8.99 (d, 1H), 8.85 (s, 1H), 7.76 (d, 1H), 7.36 (d, 1H), 6.57-6.50 (m, 2H).

### Ex. No. 115

¹H NMR (400 MHz, CDCl₃) 9.01 (d, 1H), 8.94 (s, 1H), 7.77 (d, 10H), 7.32 (d, 1H), 6.54 (dd, 1H).

### Ex. No. 116

¹H NMR (400 MHz, CDCl₃) 8.99 (d, 1H), 8.92 (s, 1H), 8.34 (d, 1H), 7.77 (d, 1H), 7.59 (d, 1H), 7.06 (dd, 1H), 6.88 (s, 1H), 6.74 (dd, 1H), 2.42 (s, 3H).

### Ex. No. 117

¹H NMR (400 MHz, CDCl₃) 9.02 (d, 1H), 8.88 (s, 1H), 8.40 (d, 10H), 7.79 (d, 10H), 7.64 (d, 1H), 7.33 (m, NH), 7.12 (dd, 1H), 6.77 (dd, 1H), 4.28 (d, 1H), 4.26 (d, 1H), 2.24 (t, 10H).

### Ex. No. 118

¹H NMR (400 MHz, CDCl₃) 9.03 (d, 1H), 8.90 (s, 1H), 8.32 (d, 10H), 7.77 (d, 1H), 7.65 (d, 1H), 7.42 (m, 2H), 7.36-7.30 (m, 1H), 7.28-7.20 (m, 3H), 6.84 (dd, 1H), 4.18 (d, 2H).

### Ex. No. 119

¹H NMR (400 MHz, CDCl₃) 9.02 (d, 1H), 8.92 (s, 1H), 8.24 (d, 1H), 7.80 (d, 1H), 7.68 (d, 1H), 7.21 (dd, 1H), 6.85 (dd, 1H), 3.26 (s, 3H).

### Ex. No. 120

¹H NMR (400 MHz, CDCl₃) 8.96 (d, 1H), 8.88 (s, 1H), 7.76 (d, 1H), 7.62 (d, 1H), 7.51 (d, 1H), 6.96 (dd, 1H), 6.83 (dd, 1H), 6.56 (dd, 1H), 6.02 (d, 1H), 5.29 (d, 1H).

### Ex. No. 121

¹H NMR (400 MHz, CDCl₃) 8.96 (d, 1H), 8.84 (s, 1H), 7.75 (d, 1H), 7.40 (d, 1H), 6.58 (d, 1H), 6.48 (dd, 1H), 2.77 (d, 3H).

### Ex. No. 122

¹H NMR (400 MHz, CDCl₃) 9.02 (d, 1H), 8.86 (s, 1H), 8.40 (d, 1H), 7.79 (d, 1H), 7.62 (d, 1H), 7.30 (m, 1H), 7.24 (m, 2H), 7.08 (dd, 1H), 6.76 (dd, 1H), 4.27 (s, 2H).

### Ex. No. 123

¹H NMR (400 MHz, CDCl₃) 9.04 (d, 1H), 8.92 (s, 1H), 8.38 (d, 1H), 7.78 (d, 1H), 7.66 (d, 1H), 7.22 (dd, 1H), 6.84 (dd, 1H), 3.82 (s, 2H), 2.66-2.60 (m, 4H), 1.72-1.62 (m, 4H), 1.48-1.41 (m, 2H).

A number of the compounds listed in Table 1 can be prepared, for example as in the following:

### Example 124

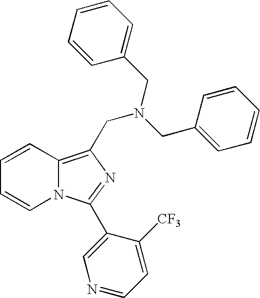

### (Subsequent Transformation)

A mixture of 21 mg (0.16 mMol, 0.5 equiv.) zinc(II) chloride (ZnCl₂) and 0.12 ml (0.62 mMol, 2 equiv.) dibenzylamine in methanol (3 ml) is stirred under argon for 5 minutes at room temperature (ca. 20° C.). With continued stirring a solution of 90 mg (0.31 mMol, 1 equiv.) 3-(4-trifluoromethylpyridin-3-yl)-imidazo[1,5-a]pyridine-1-carboxaldehyde in 2 ml methanol is then added and the mixture is stirred for 15 minutes at room temperature. 25 mg (0.40 mMol, 1.3 equiv.) sodium cyanoborohydride are then added and the resulting reaction mixture is stirred for 7 hours at room temperature. The mixture is then extracted with water/ethyl acetate and the aqueous phase is extracted further with ethyl acetate (3×50 ml). The combined organic extracts are dried with magnesium sulphate and filtered. The filtrate is evaporated under reduced pressure and the residue is worked up by column chromatography (silica-gel, ethyl acetate/hexane mixture).

150 mg (98% of theory) 1-(dibenzylaminomethyl)-3-(4-trifluoromethylpyridin-3-yl)-imidazo[1,5-a]pyridine are obtained.

¹H NMR (300 MHz, CDCl₃), 8.92 (d, 1H), 8.85 (s, 1H), 7.74 (d, 1H), 7.54 (d, 1H), 7.44-7.21 (m, 11H), 6.72 (dd, 1H), 6.54 (dd, 1H), 3.96 (s, 2H), 3.80 (s, 2H), 3.65 (s, 2H); MS (CI), m/z 473 (M++1, 100), 276 (5).

### Example 125

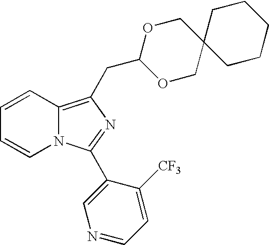

### (Subsequent Transformation)

100 mg (0.34 mMol, 1 equiv.) 3-(4-trifluoromethylpyridin-3-yl)-imidazo[1,5-a]pyridine-1-carboxaldehyde, 99 mg (0.68 mMol, 2 equiv.) 1,1-bis(hydroxymethyl)cyclohexane and catalytic quantities of toluene-4-sulphonic acid hydrate are dissolved in anhydrous toluene (10 ml) and the resulting reaction mixture is stirred for 3 hours under reflux. After cooling to room temperature the reaction mixture is extracted with water/ethyl acetate and the aqueous phase is extracted further with ethyl acetate (3×50 ml). The combined organic phases are dried with magnesium sulphate and filtered. The filtrate is evaporated under reduced pressure and the residue is purified by preparative HPLC.

34 mg (23% of theory) 1-(2′,4′-dioxaspiro[5.5]undec-3-yl)-3-(4-trifluoromethylpyridin-3-yl)imidazo[1,5-a]pyridine are obtained.

¹H NMR (300 MHz, CDCl₃), 8.95 (d, 1H), 8.84 (s, 1H), 7.92 (d, 1H), 7.74 (d, 1H), 7.52 (d, 1H), 6.81 (dd, 1H), 6.57 (dd, 1H), 5.87 (s, 1H), 4.09 (d, 2H), 3.62 (d, 2H), 1.94 (m, 2H), 1.63-1.56 (m, 2H), 1.54-1.36 (m, 4H), 1.22-1.18 (m, 2H); MS (CI), m/z 418 (M⁺+1, 100).

### Example 126

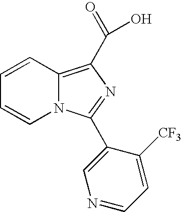

### (Subsequent Transformation)

1000 mg (3.43 mMol, 1 equiv.) 3-(4-trifluoromethylpyridin-3-yl)-imidazo[1,5-a]pyridine-1-carboxaldehyde, 758 mg (4.46 mMol, 1.3 equiv.) silver(I) nitrate and 412 mg (10.30 mMol, 3 equiv.) sodium hydroxide are added to water (30 ml) and the resulting reaction: mixture is stirred for 5 hours at room temperature. It is then filtered and the filtrate is adjusted to pH 3-4 with dilute HCl. The resulting precipitate is filtered off and dried

600 mg (54% of theory) 3-(4-trifluoromethylpyridin-3-yl)-imidazo[1,5-a]pyridine-1-carboxylic acid are obtained.

¹H NMR (300 MHz, CDCl₃), 9.04 (d, 1H), 8.88 (s, 1H), 8.32 (d, 1H), 7.84 (d, 1H), 7.66 (d, 1H), 7.24 (dd, 1H), 6.86 (dd, 1H); MS (CI), m/z 308 (M⁺+1, 100), 264 (M⁺—CO₂, 10).

### Example 127

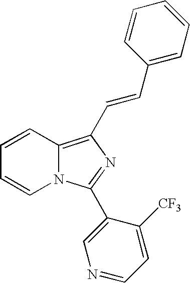

### (Subsequent Transformation)

100 mg (0.31 mMol, 1 equiv.) 3-(4-trifluoromethylpyridin-3-yl)-imidazo[1,5-a]pyridine-1-carboxaldehyde, 201 mg (0.46 mMol, 1.5 equiv.) benzylidene-triphenylphosphorane and 52 mg (0.46 mMol, 1.5 equiv.) potassium tert.-butoxide are added to anhydrous toluene (10 ml) and the resulting reaction mixture is stirred for 7 hours at room temperature. It is then extracted with water/ethyl acetate and the aqueous phase is extracted further with ethyl acetate (3×50 ml). The combined organic extracts are dried with magnesium sulphate and filtered. The filtrate is evaporated under reduced pressure and the residue is purified by column chromatography (ethyl acetate/heptane 2:1).

70 mg (55% of theory) 1-(phenylvinylidene)-3-(4-Trifluoromethylpyridin-3-yl)-imidazo[1,5-a]-pyridine are obtained as E/Z mixture.

¹H NMR (300 MHz, CDCl₃), 8.96/8.92 (d, 1H), 8.86 (s, 1H), 7.76/7.70 (d, 1H), 7.64-7.56 (m, 2H), 7.36 (d, 1H), 7.24-7.15 (m, 5H), 6.78 (d, 1H), 6.64 (dd, 1H), 6.52 (dd, 1H); MS (CI), m/z 366 (M⁺+1, 100).

### Example 128

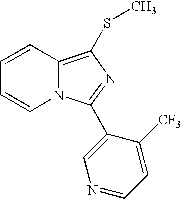

### (Subsequent Transformation)

500 mg (1.29 mMol, 1 equiv.) 1-iodo-3-(4-trifluoromethylpyridin-3-yl)-imidazo[1,5-a]pyridine are dissolved in anhydrous pyridine (10 ml) and after 5 min stirring at room temperature treated with 355 mg (1.93 mMol, 1.5 equiv.) copper bronze and 91 mg (0.96 mMol, 0.75 equiv.) dimethyldisulphide. The resulting reaction mixture is stirred for 70 hours under reflux. The reaction mixture is then treated with water, ammonium hydroxide and ammonium chloride solution and ethyl acetate, stirred for 30 min and extracted. The aqueous phase is extracted further with ethyl acetate (3×50 ml). The combined organic phases are then dried with magnesium sulphate and filtered. The filtrate is evaporated under reduced pressure and the residue is purified by preparative HPLC.

110 mg (27% of theory) 1-methylsulphanylmethyl-3-(4-trifluoromethylpyridin-3-yl)-imidazo[1,5-a]pyridine.

¹H NMR (300 MHz, CDCl₃), 8.97 (d, 1H), 8.88 (s, 1H), 7.76 (d, 1H), 7.66 (d, 1H), 7.57 (d, 1H), 6.88 (dd, 1H), 6.61 (dd, 1H), 2.54 (s, 3H); MS (ESI), m/z 310 (M⁺+1, 100).

### Example 129

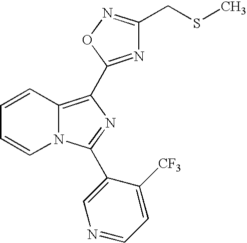

### (Subsequent Transformation)

100 mg (0.33 mMol, 1 equiv.) 3-(4-trifluoromethylpyridin-3-yl)-imidazo[1,5-a]pyridine carboxylic acid and 53 mg (0.33 mMol, 1 equiv.) 1,1′-carbonyldiimidazole are dissolved in anhydrous tetrahydrofuran (8 ml) and after 10 min stirring at 60° C. treated with 43 mg (0.36 mMol, 1.1 equiv.) thiomethylmethyleneamidoxime. The resulting reaction mixture is stirred for 4 hours at 60° C. and then 37 mg (0.33 mMol, 1 equiv.) potassium tert-butoxide is added and stirring is continued for 2 h. The reaction mixture is then treated with water and ethyl acetate and extracted. The aqueous phase is extracted further with ethyl acetate (3×50 ml). The combined organic phases are then dried with magnesium sulphate and filtered. The filtrate is evaporated under reduced pressure and the residue is purified by column chromatography.

70 mg (27% of theory) 1-(3-methylsulphanylmethyl-[1,2,4]-oxadiazol-5-yl)-3-(4-trifluoromethylpyridin-3-yl)-imidazo[1,5-a]pyridine are obtained.

¹H NMR (300 MHz, CDCl₃), 9.04 (d, 1H), 8.93 (s, 1H), 8.38 (d, 1H), 7.79 (d, 1H), 7.68 (d, 1H), 7.24 (dd, 1H), 6.86 (dd, 1H), 3.82 (s, 2H), 2.26 (s, 3H); MS (ESI), m/z 392 (M⁺+1, 100).

### Starting Materials of Structure (II):

### Example (II-14)

### N-(4,6-Dimethoxypyrimidin-2-ylmethyl)-4-trifluoromethyl-nicotinamide

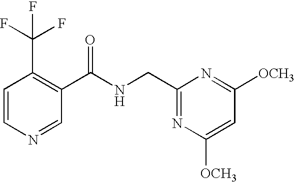

4-Trifluoromethylpyridine-3-carboxylic acid (365 mg, 1.91 mMol) was dissolved in thionyl chloride (5 ml) and treated with a catalytic amount of DMF. The reaction mixture was stirred at room temperature for 1.5 h and then at reflux for 1 h, evaporated to dryness and used in the coupling stage without purification. 4,6-Dimethoxy-2-aminomethylpyrimidine (355 mg, 2.1 mMol) in triethylamine (0.35 ml, 2.48 mMol) and dichloromethane (10 ml) was slowly treated dropwise at room temperature with a solution of the freshly prepared acid chloride (400 mg, 1.91 mMol) in dichloromethane (5 ml). The reaction mixture was stirred for 1.5 h at room temperature and then extracted with KHSO₄ solution. The combined organic phases were dried, filtered and evaporated. Subsequent purification by column chromatography gave N-(4,6-dimethoxypyrimidin-2-ylmethyl)-4-trifluoromethyl-nicotinamide (220 mg, yield: 34% of theory).

### Example (II-27)

### N-Pyrimidin-2-ylmethyl-4-trifluoromethyl-nicotinamide

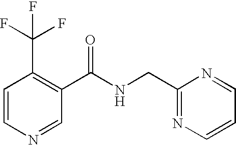

2-Cyanopyrimidine (2170 mg, 18.58 mMol) was dissolved in methanol (100 ml) and treated with conc. HCl (4.56 ml) and Pd/C (10%, water wet: 1977 mg, 1.858 mMol). Hydrogen was passed through at normal pressure for a total of 5.5 h and the course of the reaction was followed by TLC. At the end of the hydrogen passage the catalyst was filtered off, the solvent removed under reduced pressure and the residue was dried at 40° C. After NMR analysis the crude product thus obtained (HCl salt) was converted into the target product in the next step without further purification.

4-Trifluoromethylpyridine-3-carboxylic acid (5.15 g, 26.95 mMol) was suspended in anhydrous dichloromethane (50 ml) and treated with oxalyl chloride (2.907 g, 22.906 mMol) and catalytic amounts of DMF. The reaction mixture was stirred for 4 h at 40° C., evaporated to dryness and used in the coupling stage without purification.

The 2-aminomethylpyrimidine HCl salt (1.77 g, 12.157 mMol) in triethylamine (2.796 g, 27.63 mMol) and dichloromethane (10 ml) was slowly treated dropwise at room temperature with a solution of the freshly prepared acid chloride (2.316 g, 11.05 mMol) in dichloromethane (20 ml). The reaction mixture was stirred for 5 h at room temperature and then extracted with KHSO₄ solution. The combined organic phases were dried, filtered and evaporated. Subsequent purification by column chromatography gave N-pyrimidin-2-ylmethyl-4-trifluoromethyl-nicotinamide (1.40 g, yield: 45% of theory).

### Example (II-11)

### N-(4,6-Dimethylpyrimidin-2-ylmethyl)-4-trifluoromethyl-nicotinamide

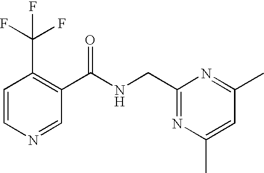

4-Trifluoromethylpyridine-3-carboxylic acid (911 mg, 4.77 mMol) was suspended in anhydrous dichloromethane (10 ml) and treated with oxalyl chloride (514 mg, 4.05 mMol) and catalytic amounts of DMF. The reaction mixture was stirred for 3.5 h under reflux, evaporated to dryness and used in the coupling stage without purification. 4,6-Dimethyl-2-aminomethylpyrimidine (786 mg, 5.73 mMol, 1.2 equiv) in triethylamine (1.0 ml, 7.16 mMol) and dichloromethane (15 ml) were slowly treated dropwise with a solution of the freshly prepared acid chloride (1000 mg, 4.77 mMol) in dichloromethane (5 ml). The reaction mixture was stirred for 3 h at room temperature and then extracted with KHSO₄ solution. The combined organic phases were dried, filtered and evaporated. Subsequent purification by column chromatography gave N-4,6-dimethylpyrimidin-2-ylmethyl)-4-trifluoromethyl-nicotinamide (800 mg, yield: 53% of theory).

### Example (II-30)

### N-(4-Chloro-6-methoxypyrimidin-2-ylmethyl)-4-trifluoromethyl nicotinamide

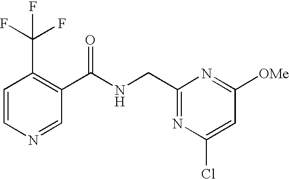

N-(4,6-Dimethoxy-pyrimidin-2-ylmethyl)-4-trifluoromethyl nicotinamide (2.0 g, 5.84 mMol) was dissolved in phosphoryl chloride (15 ml). The reaction mixture was stirred for 3.5 h at 100° C. and after cooling to room temperature partitioned between ethyl acetate and water. The combined organic phases were dried over sodium sulphate, filtered and evaporated, and the resulting crude product purified by column chromatography. N-(4-Chloro-6-methoxypyrimidin-2-ylmethyl)-4-trifluoromethyl nicotinamide (180 mg, yield 8.9% of theory) was obtained as product together with other reaction products.

### Example (II-38)

### N-(2,6-Dimethylpyrimidin-4-ylmethyl)-4-trifluoromethyl nicotinamide

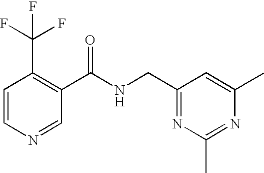

2,6-Dimethylpyrimidine-4-carbonitrile (700 mg, 5.26 mmol) was dissolved in methanol (40 ml) and treated with conc. HCl (1.10 ml) and Pd/C (10%, water wet: 559 mg, 0.53 mMol). Hydrogen was then passed through at normal pressure and the course of the reaction was followed by TLC. At the end of the hydrogen passage the catalyst was filtered off, the solvent was removed under reduced pressure and the residue dried at 40° C. After NMR analysis the 2,6-dimethylpyrimidine-4-yl-methylamine (HCl salt) thus obtained was reacted in the next step for the target product without further purification.

4-Trifluoromethylpyridine-3-carboxylic acid (2100 mg, 10.99 mmol) was suspended in anhydrous dichloromethane (10 ml) and treated with oxalyl chloride (1.185 g, 9.34 mmol) and catalytic amounts of DMF. The reaction mixture was heated under reflux for 3 h, then evaporated and a part of the crude product was used in the coupling step.

The 2,6-dimethylpyrimidin-4-ylmethylamine HCl salt (915 mg, 5.26 mmol) in triethylamine (1.68 ml, 12.03 mmol) and dichloromethane (15 ml) was slowly treated dropwise at room temperature with a solution of the freshly prepared acid chloride (1008 mg, 4.81 mmol) in dichloromethane (5 ml). The reaction mixture was stirred for 5 h at room temperature and then extracted with KHSO₄ solution. The combined organic phases were dried, filtered and evaporated. Subsequent purification by column chromatography gave N-(2,6-dimethylpyrimidin-4-ylmethyl)-4-trifluoromethyl-nicotinamide 173 mg, yield: 11% of theory).

### Example (II-29)

### N-5-Methylpyrazin-2-ylmethyl)-4-trifluoromethyl-nicotinamide

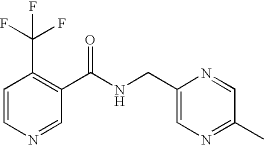

4-Trifluoromethylpyridine-3-carboxylic acid (1000 mg, 5.23 mMol) were dissolved in thionyl chloride (5.0 ml) and treated with catalytic amounts of DMF. The reaction mixture was heated under reflux for 3 h, then evaporated to dryness and a part of the crude product was used in the coupling step.

5-Methylpyrazin-2-yl-methylamine (388 mg, 3.15 mmol) in triethylamine (0.52 ml, 3.70 mmol) and dichloromethane (10 ml) was slowly treated dropwise at room temperature with a solution of the freshly prepared acid chloride (600 mg, 2.86 mmol) in dichloromethane (5 ml). The reaction mixture was stirred for 1.5 h at room temperature and then extracted with KHSO₄ solution. The combined organic phases were dried, filtered and evaporated. The subsequent purification by column chromatography gave N-(5-Methylpyrazin-2-ylmethyl)-4-trifluoromethyl-nicotinamide (106 mg, yield: 13% of theory).

The compounds of structure (II) can be prepared as above or as in the literature cited above. Examples of compounds of structure (II) are listed in Table 2.

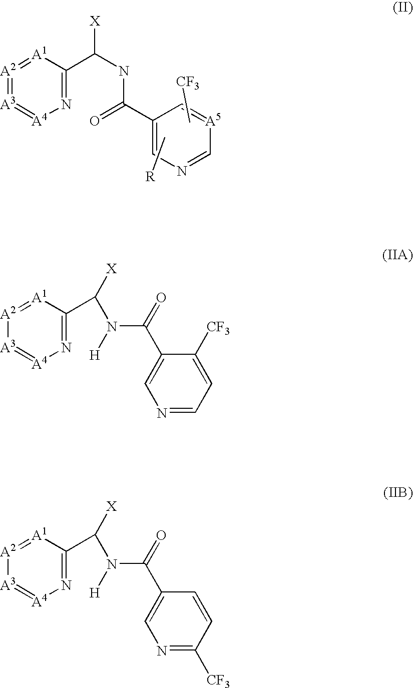

TABLE 2
Examples of compounds of the structure (II)

| Ex. | | | | | | |
|---|---|---|---|---|---|---|
| no. | Structure | X | A¹ | A² | A³ | A⁴ |
| II-1 | (IIA) | H | CH | CH | CH | CH |
| II-2 | (IIA) | H | C—Cl | CH | C—CF₃ | CH |
| II-3 | (IIA) | 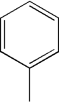 | CH | CH | CH | CH |
| II-4 | (IIA) | CH₃ | CH | CH | CH | CH |
| II-5 | (IIA) | C₃H₇-n | CH | CH | CH | CH |
| II-6 | (IIB) | 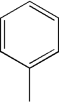 | CH | CH | CH | CH |
| II-7 | (IIB) | H | CH | CH | CH | CH |
| II-8 | (IIB) | CH₃ | CH | CH | CH | CH |
| II-9 | (IIA) | C₃H₇-n | CH | CH | CH | CH |
| II-10 | (IIA) | 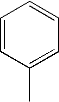 | CH | Benzo-anellation atA²-A³ | — | CH |
| II-11 | (IIA) | H | N | C—CH₃ | CH | C—CH₃ |
| II-12 | (IIB) | 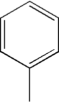 | CH | Benzo-anellation atA²-A³ | — | CH |
| II-13 | (IIB) | H | N | C—CH₃ | CH | C—CH₃ |
| II-14 | (IIA) | H | N | C—OCH₃ | CH | C—OCH₃ |
| II-15 | (IIB) | H | N | C—OCH₃ | CH | C—OCH₃ |
| II-16 | (IIA) | CH₃ | CH | Benzo- | — | CH |
| | | | | anellation at | | |
| | | | | A²-A³ | | |
| II-17 | (IIB) | CH₃ | CH | Benzo- | — | CH |
| | | | | anellation at | | |
| | | | | A²-A³ | | |
| II-18 | (IIA) | H | C—Cl | CH | C—Cl | CH |
| II-19 | (IIB) | H | C—Cl | CH | C—Cl | CH |
| II-20 | (IIA) | CH₃ | C—Cl | CH | C—CF₃ | CH |
| II-21 | (IIB) | CH₃ | C—Cl | CH | C—CF₃ | CH |
| II-22 | (IIA) | CH₃ | C—Cl | CH | C—Cl | CH |
| II-23 | (IIB) | CH₃ | C—Cl | CH | C—Cl | CH |
| II-24 | (IIB) | H | CH | N | C—CH₃ | CH |
| II-25 | (IIA) | H | CH | CH | C—CF₃ | CH |
| II-26 | (IIA) | H | CH | CH | CH | C—CF₃ |
| II-27 | (IIA) | H | N | CH | CH | CH |
| II-28 | (IIA) | H | C—Cl | CH | CH | CH |
| II-29 | (IA) | H | CH | N | C—CH₃ | CH |
| II-30 | (IA) | H | N | C—Cl | CH | C—OCH₃ |
| II-31 | (IA) | H | CH | C—C(CH₃)₃ | CH | CH |
| II-32 | (IA) | H | CH | CH | C—CH₃ | C—CH₃ |
| II-33 | (IA) | H | C—CH₃ | CH | C—CH₃ | CH |
| II-34 | (IA) | H | C—F | CH | C—F | CH |
| II-35 | (IA) | H | C—CH₃ | CH | CH | CH |
| II-36 | (IA) | H | CH | CH | C—OCH₃ | CH |
| II-37 | (IA) | H | CH | CH | C—F | CH |
| II-38 | (IA) | H | CH | C—CH₃ | N | C—CH₃ |
| II-39 | (IA) | H | N | C—C₆H₅ | CH | C—C₆H₅ |
| II-40 | (IA) | H | N | CH | CH | N |
| II-41 | (IA) | H | N | C—OCH₃ | N | C—OCH₃ |
| II-42 | (IA) | H | N | CH | N | C—Cl |
| II-43 | (IA) | H | N | C—CH₃ | N | C—CH₃ |
| II-44 | (IA) | H | N | CH | CH | C—Cl |
| II-45 | (IA) | H | N | CH | C—Cl | CH |
| II-46 | (IA) | H | N | CH | C—Br | CH |
| II-47 | (IA) | H | N | CH | C—F | CH |
| II-48 | (IA) | H | N | CH | CH | C—CH₃ |
| II-49 | (IA) | H | N | CH | C—CH₃ | C—CH₃ |
| II-50 | (IA) | H | N | CH | C-CH₃ | CH |
| II-51 | (IA) | H | N | CH | CH | C—CF₃ |
| II-52 | (IA) | H | N | CH | C—CF₃ | CH |
| II-53 | (IA) | H | N | CH | C—CH₃ | C—Cl |
| II-54 | (IA) | H | N | C—Cl | CH | C—Cl |
| II-55 | (IA) | H | N | C—Cl | CH | C—CH₃ |
| II-56 | (IA) | H | N | CH | C—CH₃ | C—Cl |
| II-57 | (IA) | H | N | C—Cl | S-Me | C—Cl |
| II-58 | (IA) | H | N | CH | C—NO₂ | CH |
| II-59 | (IA) | H | N | C—CH₃ | C—CH₃ | C—CH₃ |
| II-60 | (IA) | H | N | C—Cl | C—Cl | C—Cl |
| II-61 | (IA) | H | N | CH | C—Cl | C—Cl |
| II-62 | (IA) | H | N | CH | C—F | C—Cl |
| II-63 | (IA) | H | N | C—Br | C—Br | C—Cl |
| II-64 | (IA) | H | N | CH | CH | C—OCH₃ |
| II-65 | (IA) | H | N | CH | CH | C-OEt |
| II-66 | (IA) | H | N | CH | C₃H₇-n | CH |
| II-67 | (IA) | H | N | CH | C₂H₅ | CH |

Further physical data for the compounds Table 2:

### Ex. No. II-1

¹H NMR (400 MHz, CDCl₃), δ 8.87 (d, 1H), 8.85 (s, 1H), 8.52 (d, 10H), 7.76 (m, 1H), 7.70 (d, 1H), 7.68 (s, NH), 7.40 (d, 1H), 7.26 (dd, 1H), 4.64 (d, 2H).

### Ex. No. II-2

¹H NMR (400 MHz, CDCl₃), δ 8.90 (s, 1H), 8.88 (d, 1H), 8.82 (s, 1H), 8.16 (s, 1H), 7.70 (d, 1H), 7.65 (s, NH), 4.84 (d, 2H).

### Ex. No. II-3

¹H NMR (400 MHz, CDCl₃), δ 8.86 (s, 1H), 8.84 (d, 1H), 8.48 (d, 1H), 7.76 (m, 1H), 7.58 (d, 1H), 7.49 (s, NH), 7.36 (d, 1H), 7.18-7.32 (m, 6H), 6.26 (d, 1H).

### Ex. No. II-4

¹H NMR (400 MHz, CDCl₃), δ 8.85 (s, 1H), 8.82 (d, 1H), 8.51 (d, 1H), 7.74 (m, 1H), 7.58 (d, 1H), 7.57 (s, NH), 7.28 (d, 1H), 7.20 (dd, 1H), 5.32 (quint, 1H), 1.58 (d, 3H).

### Ex. No. II-5

¹H NMR (400 MHz, CDCl₃), δ 8.83 (s, 1H), 8.82 (d, 1H), 8.54 (d, 1H), 7.69 (m, 1H), 7.57 (d, 1H), 7.38 (d, NH), 7.28 (d, 1H), 7.19 (dd, 1H), 5.26 (q, 1H), 1.88 (m, 2H), 1.26-1.38 (m, 2H), 0.92 (t, 3H).

### Ex. No. II-6

¹H NMR (400 MHz, CDCl₃), δ 9.24 (d, 1H), 8.84 (d, NH), 8.60 (d, 1H), 8.36 (d, 1H), 7.76 (d, 1H), 7.64 (m, 1H), 7.40 (d, 2H), 7.20-7.35 (m, 5H), 6.28 (d, 1H).

### Ex. No. II-7

¹H NMR (400 MHz, CDCl₃), δ 9.22 (d, 1H), 8.58 (d, 1H), 8.36 (d, 1H), 8.08 (d, NH), 7.76 (d, 10H), 7.65 (m, 1H), 7.32 (d, 1H), 7.20 (m, 10H), 5.26 (d, 2H).

### Ex. No. II-8

¹H NMR (400 MHz, CDCl₃), δ 9.19 (d, 1H), 8.56 (d, 1H), 8.37 (d, 1H), 8.14 (d, NH), 7.76 (d, 1H), 7.62 (m, 1H), 7.30 (d, 1H), 7.22 (m, 1H), 5.30 (quint, 1H), 1.60 (d, 3H).

### Ex. No. II-9

¹H NMR (400 MHz, CDCl₃), δ 9.18 (d, 1H), 8.56 (d, 1H), 8.35 (d, 1H), 7.91 (d, NH), 7.76 (d, 1H), 7.66 (m, 1H), 7.28 (d, 1H), 7.20 (dd, 1H), 5.24 (q, 1H), 1.84 (m, 2H), 1.25 (m, 2H), 0.88 (t, 3H).

### Ex. No. II-10

¹H NMR (400 MHz, CDCl₃), δ 9.21 (s, 1H), 8.90 (s, 1H), 8.86 (d, 1H), 8.11 (d, NH), 7.96 (d, 1H), 7.82 (d, 1H), 7.70 (m, 2H), 7.60 (m, 2H), 7.45 (d, 2H), 7.29 (m, 2H), 7.24 (m, 1H, 6.54 (d, 1H).

### Ex. No. II-11

¹H NMR (400 MHz, CDCl₃), δ 8.98 (s, 1H), 8.86 (d, 1H), 7.82 (d, 1H), 7.46 (s, 1H), 6.97 (s, 1H), 4.81 (d, 2H), 2.45 (s, 6H).

### Ex. No. II-12

¹H NMR (400 MHz, CDCl₃), δ 9.27 (s, 1H), 9.22 (d, 1H), 8.62 (s, NH), 8.38 (dd, 1H), 7.98 (d, 1H), 7.78 (m, 2H), 7.70 (m, 2H), 7.60 (m, 1H), 7.44 (m, 2H), 7.22-7.35 (m, 3H), 6.51 (d, 1H).

### Ex. No. II-13

¹H NMR (400 MHz, CDCl₃), δ 9.22 (s, 1H), 8.42 (m, 2H), 7.78 (m, 1H), 7.08 (s, 1H), 4.86 (d, 2H), 2.58 (s, 6H).

### Ex. No. II-14

¹H NMR (400 MHz, CDCl₃), δ 8.98 (s, 1H), 8.88 (d, 1H), 7.62 (d, 1H), 7.17 (s, NH), 5.96 (s, 1H), 4.73 (d, 2H), 3.92 (s, 6H).

### Ex. No. II-15

¹H NMR (400 MHz, CDCl₃), δ 9.17 (s, 10H), 8.42 (d, 1H), 7.82 (d, 1H), 7.60 (s, NH), 5.98 (s, 1H), 4.75 (d, 2H), 3.97 (s, 6H).

### Ex. No. II-16

¹H NMR (400 MHz, CDCl₃), δ 8.92 (s, 10H), 8.85 (d, 1H), 8.39 (d, 1H), 8.22-8.28 (m, 2H), 7.88 (d, 1H), 7.66-7.78 (m, 2H), 7.59-7.64 (m, 2H), 6.12 (quint, 1H), 1.72 (d, 3H).

### Ex. No. II-17

¹H NMR (400 MHz, CDCl₃), δ 9.22 (s, 1H), 8.44 (d, 1H), 8.36 (d, 1H), 8.22-8.28 (m, 2H), 7.84 (d, 1H), 7.64-7.76 (m, 2H), 7.55-7.60 (m, 2H), 6.18 (quint, 1H), 1.76 (d, 3H).

### Ex. No. II-18

¹H NMR (400 MHz, CDCl₃), δ 8.95 (s, 1H), 8.88 (d, 1H), 8.40 (d, 1H), 7.78 (d, 1H), 7.61 (d, 1H), 7.46 (d, NH), 4.82 (d, 2H).

### Ex. No. II-19

¹H NMR (400 MHz, CDCl₃), δ 9.21 (d, 1H), 8.44 (d, 1H), 8.37 (dd, 1H), 7.83 (d, NH), 7.77-7.82 (m, 2H), 4.82 (d, 2H).

### Ex. No. II-20

¹H NMR (400 MHz, CDCl₃), δ 8.88 (d, 1H), 8.86 (d, 1H), 8.69 (s, 1H), 7.99 (s, 1H), 7.62 (d, 1H), 7.48 (d, NH), 5.80 (quint, 1H), 1.58 (d, 3H).

### Ex. No. II-21

¹H NMR (400 MHz, CDCl₃), δ 9.16 (s, 1H), 8.76 (s, 1H), 8.35 (dd, 1H), 7.99 (s, 1H), 7.82 (d, NH), 7.78 (d, 1H), 5.78 (quint, 1H), 1.60 (d, 3H).

### Ex. No. II-22

¹H NMR (400 MHz, CDCl₃), δ 8.86 (d, 1H), 8.85 (d, 1H), 8.38 (d, 1H), 7.77 (d, 1H), 7.59 (d, 1H), 7.40 (d, NH), 5.69 (quint, 1H), 1.56 (d, 3H).

### Ex. No. II-23

¹H NMR (400 MHz, CDCl₃), δ 9.15 (d, 1H), 8.44 (d, 1H), 8.34 (dd, 1H), 7.82 (d, NH), 7.76-7.81 (m, 2H), 5.68 (quint, 1H), 1.56 (d, 3H).

### Ex. No. II-24

¹H NMR (400 MHz, CDCl₃), δ 9.16 (d, 1H), 8.58 (d, 1H), 8.42 (d, 1H), 8.35 (dd, 1H), 7.79 (d, 1H), 7.44 (s, NH), 4.80 (d, 2H), 2.60 (s, 3H).

### Ex. No. II-25

¹H NMR (400 MHz, CDCl₃), δ 8.94 (s, 1H), 8.90 (d, 1H), 8.81 (d, 1H), 7.96 (dd, 1H), 7.62 (d, 1H), 7.51 (d, 1H), 7.34 (s, NH), 4.85 (d, 2H).

### Ex. No. II-26

¹H NMR (400 MHz, CDCl₃), δ 8.95 (s, 1H), 8.89 (d, 1H), 7.90 (t, 1H), 7.56-7.64 (m, 3H), 7.22 (s, 1H), 4.84 (d, 2H).

### Ex. No. II-27

¹H NMR (400 MHz, CDCl₃), δ 8.98 (s, 1H), 8.89 (d, 1H), 8.74 (d, 2H), 7.62 (d, 1H), 7.34 (s, NH), 7.25 (t, 1H), 4.92 (d, 2H).

### Ex. No. II-28

¹H NMR (400 MHz, CDCl₃), δ 8.97 (s, 1H), 8.88 (d, 1H), 8.42 (dd, 1H), 7.72 (dd, 1H), 7.68 (s, NH), 7.62 (d, 1H), 7.22 (m, 1H), 4.84 (d, 2H).

### Ex. No. II-29

¹H NMR (400 MHz, CDCl₃), δ 8.88 (s, 1H), 8.86 (d, 1H), 8.57 (s, 1H), 8.37 (s, 1H), 7.59 (d, 1H), 7.04 (s, NH), 4.78 (d, 2H), 2.58 (s, 3H).

### Ex. No. II-30

¹H NMR (400 MHz, CDCl₃), δ 8.99 (s, 1H), 8.89 (d, 1H), 7.64 (d, 1H), 7.05 (s, NH), 6.68 (s, 1H), 4.79 (d, 2H), 4.01 (s, 3H).

### Ex. No. II-31

¹H NMR (400 MHz, CDCl₃), δ 8.99 (s, 1H), 8.96 (d, 1H), 8.47 (d, 1H), 7.66 (d, 1H), 7.54 (s, NH), 7.39 (s, 1H), 7.34 (d, 1H), 4.84 (d, 2H), 1.41 (s, 9H).

### Ex. No. II-32

¹H NMR (400 MHz, CDCl₃), δ 8.98 (s, 1H), 8.93 (d, 1H), 8.32 (d, NH), 7.66-7.62 (m, 2H), 7.34 (d, 1H), 4.82 (d, 2H), 2.62 (s, 3H), 2.40 (s, 3H).

### Ex. No. II-33

¹H NMR (400 MHz, CDCl₃), δ 8.95 (s, 1H), 8.86 (d, 1H), 8.15 (s, 1H), 8.04 (s, NH), 7.60 (d, 1H), 7.36 (s, 1H), 4.65 (d, 2H), 2.33 (s, 6H).

### Ex. No. II-34

¹H NMR (400 MHz, CDCl₃), δ 8.94 (s, 1H), 8.87 (d, 1H), 8.26 (s, 1H), 7.61 (d, 1H), 7.29-7.19 (m, 1H+NH), 4.84 (d, 2H).

### Ex. No. II-35

¹H NMR (400 MHz, CDCl₃), δ 8.96 (s, 1H), 8.87 (d, 1H), 8.35 (d, 1H), 8.07 (s, NH), 7.62 (d, 1H), 7.54 (d, 1H), 7.17 (dd, 1H), 4.72 (d, 2H), 2.36 (s, 3H).

### Ex. No. II-36

¹H NMR (400 MHz, CDCl₃), δ 8.89 (s, 1H), 8.84 (d, 1H), 8.19 (d, 1H), 7.58 (d, 1H), 7.29-7.20 (m, 2H+NH), 4.68 (d, 2H), 3.84 (s, 3H).

### Ex. No. II-37

¹H NMR (400 MHz, CDCl₃), δ 8.95 (s, 1H), 8.88 (d, 1H), 8.36 (d, 1H), 7.61 (d, 1H), 7.50-7.45 (m, 2H), 7.32-7.25 (m, 1H), 4.86 (d, 2H).

### Ex. No. II-38

¹H NMR (400 MHz, CDCl₃), δ 8.96 (s, 1H), 8.89 (d, 1H), 7.63 (d, 1H), 7.30 (s, NH), 7.02 (s, 1H), 4.67 (d, 2H), 2.66 (s, 3H), 2.52 (s, 3H).

### Ex. No. II-39

¹H NMR (400 MHz, CDCl₃), δ 9.04 (s, 1H), 8.92 (d, 1H), 8.16-8.10 (m, 4H), 8.02 (s, 1H), 7.64 (d, 1H), 7.58-7.52 (m, 6H+NH), 5.04 (d, 2H).

### BIOLOGICAL EXAMPLES

### Example A

Aphis gossypii test

| | |
|---|---|
| Solvent: | 7 parts by weight dimethylformamide |
| Emulsifier: | 2 parts by weight alkylarylpolyglycol ether |

To prepare a suitable active material preparation 1 part by weight of active compound is mixed with the given amount of solvent and emulsifier and the concentrate with water/emulsifier is diluted to the desired concentration.

Cotton leaves (Gossypium hirsutum) that are strongly infested with the cotton aphid (Aphis gossypii) were treated by immersion in the active material preparation at the desired concentration

After the desired time the death rate was determined in %, where 100% means that all aphids were killed; 0% means that no aphids were killed

In this test, for example, the compounds of preparation examples 1, 4, 5, 6, 8, 10, 13, 22, 30, 32, 33, 34, 35, 36, 38, 39, 41, 44, 45, 46, 47, 48, 49, 50, 52, 53, 54, 57, 60, 61, 62, 63, 64, 65, 66, 67, 68, 70, 71, 72, 73, 75, 76, 77, 79, 80, 81, 84, 86, 87, 90, 91, 92, 93, 94, 95, 96, 97, 98, 99, 100, 101, 102, 104, 105, 106, 107, 108, 109, 111, 112, 114, 117, 121, 123, 124, 125, 126, 127 and II-33, II-34, II-35, II-36, II-37 showed a death rate of at least 80% after 6 days at a concentration of 100 ppm.

### Example B

Myzus test (spray treatment)

| | | |
|---|---|---|
| Solvent: | 78 | parts by weight acetone |
| | 1.5 | parts by weight dimethylformamide |
| Emulsifier: | 0.5 | parts by weight alkylarylpolyglycol ether |

To prepare a suitable active material preparation 1 part by weight of active compound is mixed with the given amount of solvent and emulsifier and the concentrate with water/emulsifier is diluted to the desired concentration.

China cabbage leaves (Brassica pekinensis) that were infested with all stages of the green peach aphid (Myzus persicae) were sprayed with the active compound preparation at the desired concentration.

After the desired time the death rate was determined in %, where 100% means that all aphids were killed; 0% means that no aphids were killed.

In this test, for example, the compounds of preparation examples 1, 3, 4, 5, 6, 7, 8, 10, 11, 12, 13, 14, 15, 16, 17, 19, 20, 21, 22, 23, 24, 25, 26, 27, 29, 30, 32, 33, 34, 35, 36, 38, 39, 40, 41, 42, 44, 45, 46, 47, 48, 49, 50, 51, 52, 53, 54, 55, 57, 58, 59, 60, 61, 62, 63, 64, 65, 67, 68, 69, 70, 71, 72, 73, 74, 75, 76, 78, 79, 80, 82, 83, 84, 85, 86, 87, 88, 89, 90, 91, 92, 93, 94, 95, 96, 97, 98, 99, 100, 101, 103, 104, 105, 106, 107, 108, 109, 110, 111, 112, 113, 114, 115, 116, 117, 119, 121, 122, 123, 124, 125, 126, 127, 128, II-31, II-32, II-33, II-34, II-36 and II-38 showed a death rate of at least 80% after 5 days at a concentration of 500 g/ha.

### Example C

### In Vitro Test for ED₅₀ Determination with Micro-Organisms

A methanolic solution of the investigation compound treated with the emulsifier PS16 was pipetted into the wells of microtiter plates. After the solvent had evaporated 200 μl potato-dextrose medium were added to each well. The medium had been treated previously with a suitable concentration of spores or mycelles of the fungus under investigation. The resulting concentrations of the active compound were 0.1, 1, 10 and 100 ppm. The resulting concentration of the emulsifier was 300 ppm. The plates were then incubated on a shaker 3-5 days at a temperature of 22° C. until an adequate growth was determined in the untreated control. Evaluation was carried out photometrically at a wave length of 620 nm. The active compound dose that lead to a 50% inhibition of fungal growth compared with the untreated control (ED₅₀) was calculated from the measurement data of the different concentrations.

### Example C

In vitro test for ED₅₀ determination with micro-organisms

| Active compound | Micro-organism | ED₅₀ value |
|---|---|---|
| 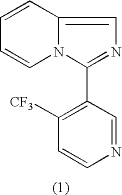 | Ustilago avenae | <0.1 |
| 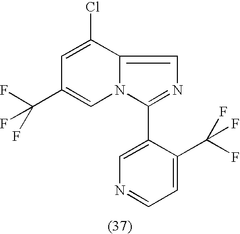 | Ustilago avenae | <0.1 |
| 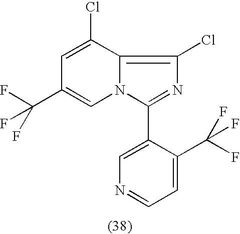 | Ustilago avenae | <0.1 |

### Example D

Nilaparvata lugens test (hydroponic treatment)

| | | |
|---|---|---|
| Solvent: | 78 | parts by weight acetone |
| | 1.5 | parts by weight dimethylformamide |
| Emulsifier: | 0.5 | parts by weight alkylarylpolyglycol ether |

To prepare a suitable active material preparation 1 part by weight of active compound is mixed with the given amount of solvent and emulsifier and the concentrate with water/emulsifier is diluted to the desired concentration.

The active compound preparation was pipetted into water. The concentration given refers to the amount of active compound per volume unit water (mg/l=ppm). Infection was then carried out with the brown rice plant hopper (Nilaparvata lugens).

After the desired time the death rate was determined in %, where 100% means that all rice hoppers had been killed; 0% means that no rice hopper had been killed

In this test, for example the compounds of preparation examples 3, 4, 16, 17, 23, 26, 54, 55, 100, 106 and 124 showed a death rate of at least 70% after 7 days at a concentration of 100 g/ha.

### BIOLOGICAL EXAMPLES FOR COMPOUNDS OF STRUCTURE (II-b)

### Example A

Myzus test (spray treatment)

| | | |
|---|---|---|
| Solvent: | 78 | parts by weight acetone |
| | 1.5 | parts by weight dimethylformamide |
| Emulsifier: | 0.5 | parts by weight alkylarylpolyglycol ether |

To prepare a suitable active material preparation 1 part by weight of active compound is mixed with the given amount of solvent and emulsifier and the concentrate with water/emulsifier is diluted to the desired concentration.

China cabbage leaves (Brassica pekinensis) that were infested with all stages of the green peach aphid (Myzus persicae) were sprayed with the active compound preparation at the desired concentration.

After the desired time the death rate was determined in %, where 100% means that all aphids were killed; 0% means that no aphids were killed.

In this test, for example, the compounds of preparation examples II-11, II-27, II-29 and II-38 showed a death rate of at least 90% after 5 days at a concentration of 500 g/ha.

What is claimed is:

1-21. (canceled)

22: An azinylimidazoazine of structure (I) 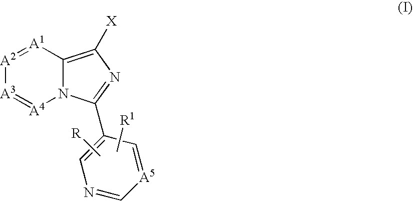
and/or a salt and/or N-oxide thereof,
wherein
A¹, A², A³, A⁴, and A⁵ are the same or different and in each case represent N (nitrogen) or the group C—R, with the provisos that the imidazoazine bicycle must contain 2 to 5 N atoms and no more than two N atoms are next to each other,
R in each case independently represents H (hydrogen), nitro, amino, cyano, or halogen; or represents optionally substituted alkyl, alkoxy, alkylthio, alkylsulphinyl, alkylsulphonyl, alkylamino, or dialkylamino; or two neighboring R groups optionally together represent alkanediyl or together with the azine group to which they are connected form a benzene ring,
R¹ represents (C₁-C₄)-haloalkyl, and
X represents H (hydrogen), nitro, formyl, hydroximinomethyl (—CH═N—OH), aminoiminomethyl (—CH═N—NH2), amino, cyano, or halogen; or represents optionally substituted —COOH, aminocarbonyl (—CO—NH₂), alkyl, alkylcarbonyl, alkoxy, alkoxycarbonyl, alkoximinomethyl (—CH═N—O-alkyl), alkylaminoiminomethyl (—CH═N—NH-alkyl), dialkylaminoiminomethyl, cycloalkylalkoxyiminomethyl, benzyloxyiminomethyl, alkenyloxyiminomethyl, arylsulphonylaminoiminomethyl, alkylcarbonyloxyiminomethyl, alkylthio, alkylsulphinyl, alkylsulphonyl, alkylamino, aminocarbonyl, hydroxycarbonyl alkylaminocarbonyl, alkenylaminocarbonyl, alkynylaminocarbonyl, dialkylamino, dialkylaminocarbonyl, alkylcarbonylaminocarbonyl, N-alkylalkylcarbonylaminocarbonyl, alkoxycarbonylaminocarbonyl, N-alkylalkoxycarbonylaminocarbonyl, alkylaminocarbonylaminocarbonyl, N-alkyl-N-alkylaminocarbonylaminocarbonyl, alkenyl, alkenyloxy, alkenylamino, alkenyloximinomethyl, alkynyl, alkynyloxy, alkynylamino, cycloalkyl, cycloalkyloxy, cycloalkylalkoximinomethyl, cycloalkylamino, cycloalkylalkyl, cycloalkylalkoxy, cycloalkylalkylamino, aryl, aryloxy, arylthio, arylamino, arylaminoiminomethyl, arylalkyl, arylethynyl, arylalkoxy, arylalkylthio, arylalkylamino, arylalkylaminoiminomethyl, arylalkoxyiminomethyl, arylsulphonylaminoiminomethyl, heterocyclyl, heterocyclyloxy, heterocyclylthio, heterocyclylamino, heterocyclylalkyl, heterocyclylalkynyl, heterocyclylalkoxy, heterocyclylalkylthio, or heterocyclylalkylamino.

23: An azinylimidazoazine according to claim 22, wherein
A¹, A², A³, A⁴, and A⁵ are the same or different and represent N (nitrogen) or the group C—R, with the provisos that the imidazoazine bicycle must contain 2 to 5 N atoms and no more than two N atoms are next to each other,
R in each case independently represents H (hydrogen), nitro, amino, cyano, or halogen; or represents alkyl, alkoxy, alkylthio, alkylsulphinyl, alkylsulphonyl, alkylamino, or dialkylamino in each case having 1 to 6 carbon atoms in the alkyl groups and in each case optionally substituted by cyano, halogen, or C₁-C₄-alkoxy; or two neighboring R groups optionally together represent alkanediyl having 3 to 5 carbon atoms or together with the azine group to which they are connected form a benzene ring,
R¹ represents CF₃, CHF₂, or CF₂Cl, and
X represents H (hydrogen), hydroxycarbonyl (COOH), nitro, formyl, hydroximinomethyl (—CH═N—OH), aminoiminomethyl (—CH═N—NH2), amino, cyano, or halogen; represents alkyl having 1 to 6 carbon atoms and optionally substituted by cyano, hydroxy, halogen, C₁-C₄-alkoxy, C₁-C₄-alkylamino, di(C₁-C₄-alkyl)aminocarbonyloxy, C₁-C₄-alkylcarbonyloxy, benzylamino, dibenzylamino, pyrrolidinyl; piperidinyl (which is itself optionally substituted by C₁-C₄-haloalkyl), morpholinyl (which is itself optionally substituted by C₁-C₄-alkyl)piperazinyl, N-methylpiperazinyl, or di(C₁-C₄-alkyl)amino; represents aminocarbonyl optionally substituted by benzyloxycarbonyl or N,O-di(C₁-C₄-alkyl)hydroxylaminocarbonyl; represents alkylcarbonyl, alkoxy, alkoxycarbonyl, alkoximinomethyl (—CH═N—O-alkyl), alkylaminoiminomethyl (—CH═N—NH-alkyl), dialkylaminoiminomethyl, benzyloxyiminomethyl, C₂-C₅-alkenyloxyiminomethyl, phenylsulphonylaminoiminomethyl, alkylcarbonyloxyiminomethyl, alkylthio, alkylsulphinyl, alkylsulphonyl, alkylamino, alkylaminocarbonyl, dialkylamino, dialkylaminocarbonyl, alkylcarbonylaminocarbonyl, N-alkylalkylcarbonylaminocarbonyl, alkoxycarbonylaminocarbonyl, N-alkylalkoxycarbonylaminocarbonyl, alkylaminocarbonylaminocarbonyl, or N-alkyl-N-alkylaminocarbonylaminocarbonyl in each case having 1 to 6 carbon atoms in the alkyl groups and optionally substituted by cyano, hydroxy, halogen, C₁-C₄-alkoxy, C₁-C₄-alkoxycarbonyl, benzyloxycarbonyl, or N,O-dialkylhydroxylaminocarbonyl; represents alkenyl, alkenyloxy, alkenylamino, alkenylaminocarbonyl, alkenyloximinomethyl, alkynyl, alkynyloxy, alkynylaminocarbonyl, or alkynylamino in each case having 2 to 8 carbon atoms in the alkenyl or alkynyl groups and optionally substituted by cyano, hydroxy, C₁-C₆-alkoxy, phenyl (which itself is optionally substituted by nitro, amino, hydroxy, cyano, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulphinyl, C₁-C₄-halogenalkylsulphinyl, C₁-C₄-alkylsulphonyl, C₁-C₄-haloalkylsulphonyl, C₁-C₄-alkylamino, di(C₁-C₄-alkyl)amino, di(C₁-C₄-alkyl)aminocarbonyl, or di(C₁-C₄-alkyl)aminosulphonyl), phenoxy, heterocyclyl (in each case having up to 8 carbon atoms and at least one heteroatom selected from the series N (nitrogen), O (oxygen), and S (sulphur) and optionally also a group CO, CS, SO, or SO₂ as a ring component of the heterocycle, which is itself optionally substituted by halogen or C₁-C₄-alkyl), C₁-C₄-alkoxycarbonyl, benzyloxycarbonyl, N,O-di(C₁-C₄-alkyl)aminocarbonyl, trialkylsilyl, or halogen; represents cycloalkyl, cycloalkyloxy, cycloalkylalkoximinomethyl, cycloalkylamino, cycloalkylalkyl, cycloalkylalkoxy, or cycloalkylalkylamino in each case having 3 to 6 carbon atoms in the cycloalkyl groups and optionally 1 to 4 carbon atoms in the alkyl groups and optionally substituted by cyano, halogen, C₁-C₄-alkyl, or C₁-C₄-haloalkyl; represents aryl, aryloxy, arylthio, arylamino, arylaminoiminomethyl, arylalkyl, arylethynyl, arylalkoxy, arylalkylthio, arylalkylamino, arylalkylaminoiminomethyl, arylalkoxyiminomethyl, or arylsulphonylaminoiminomethyl in each case having 6 or 10 carbon atoms in the aryl group and optionally 1 to 4 carbon atoms in the alkyl group and optionally substituted by nitro, amino, hydroxy, cyano, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulphinyl, C₁-C₄-haloalkylsulphinyl, C₁-C₄-alkylsulphonyl, C₁-C₄-Haloalkylsulphonyl, C₁-C₄-alkylamino, di(C₁-C₄-alkyl)-amino, di(C₁-C₄-alkyl)-aminocarbonyl, di(C₁-C₄-alkyl)aminosulphonyl, phenoxy, or phenyl; or represents heterocyclyl, heterocyclyloxy, heterocyclylthio, heterocyclylamino, heterocyclylalkyl, heterocyclylalkynyl, heterocyclylalkoxy, heterocyclylalkylthio, or heterocyclylalkylamino in each case having up to 8 carbon atoms and at least one heteroatom selected from the series N (nitrogen), O (oxygen), and S (sulphur) and optionally also a group CO, CS, SO, or SO₂ as a ring component of the heterocycle and optionally up to 4 carbon atoms in the alkyl group and alkynyl group and in each case optionally substituted by nitro, amino, hydroxy, cyano, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-alkylthio-C₁-C₄-alkyl, C₁-C₄-haloalkylthio, C₁-C₄-alkylsulphinyl, C₁-C₄-haloalkylsulphinyl, C₁-C₄-alkylsulphonyl, C₁-C₄-haloalkylsulphonyl, C₁-C₄-alkylamino, di(C₁-C₄-alkyl)amino, di(C₁-C₄-alkyl)aminocarbonyl, di(C₁-C₄-alkyl)aminosulphonyl, benzyl, thienylsulphonylmethyl, piperidinomethyl, or phenyl; or represents 2,4-dioxaspiro[5.5]undec-8-en-3-yl or 2,4-dioxaspiro[5.5]undecan-3-yl.

24: An azinylimidazoazine according to claim 22, wherein
A¹, A², A³, A⁴, and A⁵ are the same or different and represent N (nitrogen) or the group C—R, with the provisos that the imidazoazine bicycle must contain 2 to 4 N atoms and no more than two N atoms are next to each other,
R in each case independently represents H (hydrogen), nitro, amino, cyano, fluorine, chlorine, bromine, or iodine; or represents methyl, ethyl, n- or i-propyl, n-, i-, s-, or t-butyl, methoxy, ethoxy, n- or i-propoxy, n-, i-, s-, or t-butoxy, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, methylamino, ethylamino, n- or i-propylamino, n-, i-, s-, or t-butylamino, dimethylamino or diethylamino, in each case optionally substituted by cyano, fluorine, chlorine, bromine, methoxy, n- or i-propoxy, n-, i-, s-, or t-butoxy; or two neighboring R groups optionally together represent propan-1,3-diyl, butane-1,3-diyl, butane-1,4-diyl, pentane-1,3-diyl, pentane-1,4-diyl, or pentane-1,5-diyl or together with the azine group to which they are connected form a benzene ring,
R¹ represents CF₃, and
X represents H (hydrogen) or hydroxycarbonyl (COOH); represents aminocarbonyl optionally substituted by benzyloxycarbonyl, N,O-bimethylhydroxylaminocarbonyl, N,O-biethylhydroxylaminocarbonyl, N-methyl-O-ethylhydroxylaminocarbonyl, or N-ethyl-O-methylhydroxylaminocarbonyl; represents nitro, formyl, hydroximinomethyl (—CH═N—OH), aminoiminomethyl (—CH═N—NH₂), amino, cyano, fluorine, chlorine, bromine, or iodine; represents methyl, ethyl, n- or i-propyl, n-, i-, s-, or t-butyl, or n-, i-, s-, t-, or neo-pentyl, in each case optionally substituted by cyano, hydroxy, fluorine, chlorine, bromine, methoxy, ethoxy, n- or i-propoxy, n-, i-, s-, or t-butoxy, benzyloxy, methylamino, ethylamino, n- or i-propylamino, n-, i-, s-, or t-butylamino, dimethylaminocarbonyloxy, diethylaminocarbonyloxy, methylcarbonyloxy, ethylcarbonyloxy, benzylamino, dibenzylamino, dimethylamino, diethylamino, or dipropylamino; represents methyl substituted by the group NR′R″ (where R′R″ together with the nitrogen atom represent pyrrolidine, piperidine, 4-trifluoromethylpiperidine, 3-trifluoromethylpiperidine, fluoromethylpiperidine, morpholine, dimethylmorpholine, piperazine, or N-methylpiperazine); represents acetyl, propionyl, n- or i-butyroyl, methoxy, ethoxy, n- or i-propoxy, n-, i-, s-, or t-butoxy, methoxycarbonyl, ethoxycarbonyl, n- or i-propoxycarbonyl, methoximinomethyl, ethoximinomethyl, methylaminoiminomethyl, ethylaminoiminomethyl, n- or i-propylaminoiminomethyl, dimethylaminoiminomethyl, cyclohexylmethoxyiminomethyl, cycloentylmethoxyiminomethyl, cyclopropylmethoxyiminomethyl, benzyloxyiminomethyl, chlorobenzyloxyiminomethyl, ethylcarbonyloxyiminomethyl, methylcarbonyloxyiminomethyl, allyloxyiminomethyl, phenylsulphonylaminoiminomethyl, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s-, or t-butylamino, methylaminocarbonyl, ethylaminocarbonyl, n- or i-propylaminocarbonyl, dimethylamino, diethylamino, di-n-propylamino, di-i-propylamino, dimethylaminocarbonyl, diethylaminocarbonyl, acetylaminocarbonyl, propionylaminocarbonyl, n- or i-butyroylaminocarbonyl, N-methyl-acetylaminocarbonyl, N-methyl-propionylaminocarbonyl, methoxycarbonylaminocarbonyl, ethoxycarbonylaminocarbonyl, n- or i-propoxycarbonylaminocarbonyl, N-methylmethoxycarbonylaminocarbonyl, N-methylethoxycarbonylaminocarbonyl, methylaminocarbonylaminocarbonyl, ethylaminocarbonylaminocarbonyl, n- or i-propylamino-carbonylaminocarbonyl, N-methyl-methylaminocarbonylaminocarbonyl, N-methyl-ethylaminocarbonylamino, N,O-dimethylhydroxylaminocarbonyl, N,O-diethylhydroxylaminocarbonyl, N-methyl-O-ethylhydroxylaminocarbonyl, or N-ethyl-O-methylhydroxylaminocarbonyl, in each case optionally substituted by cyano, hydroxy, fluorine, chlorine, bromine, methoxy, ethoxy, n- or i-propoxy, or n-, i-, s-, or t-butoxy; represents ethenyl, propenyl, butenyl, pentenyl, hexenyl, propenyloxy, butenyloxy, pentenyloxy, propenylamino, butenylamino, pentenylamino, allyloximinomethyl, ethynyl, propynyl, butynyl, pentynyl, hexynyl, heptynyl, propynyloxy, butynyloxy, pentynyloxy, propynylaminocarbonyl, butynylaminocarbonyl, or pentynylaminocarbonyl, in each case optionally substituted by cyano, hydroxy, methoxy, ethoxy, n- or i-propoxy, n-, i-, s-, or t-butoxy, phenyl (which is itself optionally substituted by C₁-C₄-alkyl, C₁-C₄-alkoxy, halogen, or C₁-C₄-haloalkyl), phenoxy, heterocyclyl (wherein the heterocyclyl is selected from the group consisting of furyl, tetrahydrofuryl, thienyl, pyrrolyl, pyrrolinyl, pyrrolidinyl, pyrazolyl, pyrazolinyl, oxazolyl, oxazolinyl, isoxazolyl, isoxazolinyl, thiazolyl, isothiazolyl, oxadiazolyl, thiadiazolyl, pyridinyl, pyrrolidinyl, morpholinyl, piperazinyl, or pyrimidinyl, each of which is optionally substituted by halogen or C₁-C₄-alkyl), trialkylsilyl, ethoxycarbonyl, methoxycarbonyl, fluorine, chlorine, or bromine; represents cyclopropyl, cyclobutyl, cyclopentyl, cyclohexyl, cyclopropyloxy, cyclobutyloxy, cyclopentyloxy, cyclohexyloxy, cyclopropylamino, cyclobutylamino, cyclopentylamino, cyclohexylamino, cyclopropylmethyl, cyclobutylmethyl, cyclopentylmethyl, cyclohexylmethyl, cyclopropylmethoxy, cyclobutylmethoxy, cyclopentylmethoxy, cyclohexylmethoxy, cyclopropylmethoximinomethyl, cyclopropylmethylamino, cyclobutylmethylamino, cyclopentylmethylamino, or cyclohexylmethylamino, in each case optionally substituted by cyano, fluorine, chlorine, bromine, iodine, methyl, ethyl, n- or i-propyl, n-, i-, s-, or t-butyl, fluoromethyl, chloromethyl, difluoromethyl, dichloromethyl, trifluoromethyl, or trichloromethyl; represents phenyl, naphthyl, phenoxy, naphthyloxy, phenylthio, naphthylthio, phenylamino, naphthylamino, phenylaminoiminomethyl, benzyl, phenylethyl, phenylpropyl, phenylethynyl, phenylmethoxy, phenylethoxy, phenylpropoxy, phenylmethylthio, phenylmethylamino, phenylethylamino, phenylmethylaminoiminomethyl, phenylmethoxyiminomethyl, or phenylsulphonylaminoiminomethyl, in each case optionally substituted by nitro, amino, hydroxy, cyano, carbamoyl, thiocarbamoyl, fluorine, chlorine, bromine, iodine, methyl, ethyl, n- or i-propyl, n-, i-, s-, or t-butyl, fluoromethyl, chloromethyl, difluoromethyl, dichloromethyl, trifluoromethyl, trichloromethyl, fluorodichloromethyl, chlorodifluoromethyl, methoxy, ethoxy, n- or i-propoxy, n-, i-, s-, or t-butoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, n-, i-, s-, or t-butylthio, difluoromethylthio, trifluoromethylthio, chlorodifluoromethylthio, methylsulphinyl, ethylsulphinyl, n- or i-propylsulphinyl, trifluoromethylsulphinyl, methylsulphonyl, ethylsulphonyl, trifluoromethylsulphonyl, methylamino, ethylamino, n- or i-propylamino, n-, i-, s-, or t-butylamino, dimethylamino, diethylamino, dimethylaminocarbonyl, diethylaminocarbonyl, dimethylaminosulphonyl, diethylaminosulphonyl, phenoxy, or phenyl; represents heterocyclyl, heterocyclyloxy, heterocyclylthio, heterocyclylamino, heterocyclylmethyl, heterocyclylethynyl, heterocyclylmethoxy, heterocyclylmethylthio, or heterocyclylmethylamino, where heterocyclyl in each case represents furyl, tetrahydrofuryl, thienyl, pyrrolyl, pyrrolinyl, pyrrolidinyl, pyrazolyl, pyrazolinyl, oxazolyl, oxazolinyl, isoxazolyl, isoxazolinyl, thiazolyl, isothiazolyl, oxadiazolyl, thiadiazolyl, dioxolanyl, dioxanyl, pyridinyl, piperidinyl, morpholinyl, pyrimidinyl, or piperazinyl, in each case optionally substituted by nitro, amino, hydroxy, cyano, carbamoyl, thiocarbamoyl, fluorine, chlorine, bromine, iodine, methyl, ethyl, n- or i-propyl, n-, i-, s-, or t-butyl, fluoromethyl, chloromethyl, difluoromethyl, dichloromethyl, trifluoromethyl, trichloromethyl, fluorodichloromethyl, chlorodifluoromethyl, methoxy, ethoxy, n- or i-propoxy, n-, i-, s-, or t-butoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, n-, i-, s-, or t-butylthio, methylthiomethyl, ethylthiomethyl, difluoromethylthio, trifluoromethylthio, chlorodifluoromethylthio, methylsulphinyl, ethylsulphinyl, n- or i-propylsulphinyl, trifluoromethylsulphinyl, methylsulphonyl, ethylsulphonyl, trifluoromethylsulphonyl, methylamino, ethylamino, n- or i-propylamino, n-, i-, s-, or t-butylamino, dimethylamino, diethylamino, dimethylaminocarbonyl, diethylaminocarbonyl, dimethylaminosulphonyl, diethylaminosulphonyl, thienylsulphonylmethyl, piperidinomethyl, benzyl, or phenyl; or represents 2,4-dioxaspiro[5.5]undec8-en-3-yl or 2,4-dioxaspiro[5.5]undecan-3-yl.

25: An azinylimidazoazine according to claim 22, wherein
A¹, A², A³, A⁴, and A⁵ are the same or different and represent N (nitrogen) or the group C—R, with the proviso that the imidazoazine bicycle contains 2 or 3 N atoms,
R in each case independently represents H (hydrogen), nitro, amino, cyano, fluorine, chlorine, bromine, or iodine; represents methyl, ethyl, n- or i-propyl, n-, i-, s-, or t-butyl, methoxy, ethoxy, n- or i-propoxy, n-, i-, s-, or t-butoxy, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, methylamino, ethylamino, n- or i-propylamino, n-, i-, s-, or t-butylamino, dimethylamino, or diethylamino, in each case optionally substituted by cyano, fluorine, chlorine, bromine, methoxy, ethoxy, n- or i-propoxy, n-, i-, s-, or t-butoxy; or two neighboring R groups optionally together represent propane-1,3-diyl, butane-1,3-diyl, butane-1,4-diyl, pentane-1,3-diyl, pentane-1,4-diyl, or pentane-1,5-diyl or together with the azine group to which they are connected form a benzene ring,
R¹ represents CF₃, and
X represents H (hydrogen) or hydroxycarbonyl (COOH); represents aminocarbonyl optionally substituted by benzyloxycarbonyl, N,O-dimethylhydroxylaminocarbonyl, N,O-diethylhydroxylaminocarbonyl, N-methyl-O-ethylhydroxylaminocarbonyl, or N-ethyl-O-methylhydroxylaminocarbonyl; represents nitro, formyl, hydroximinomethyl, aminoiminomethyl, amino, cyano, fluorine, chlorine, bromine, or iodine; represents methyl, ethyl, n- or i-propyl, n-, i-, s-, or t-butyl, or n-pentyl, in each case optionally substituted by cyano, hydroxy, fluorine, chlorine, methoxy, ethoxy, n- or i-propoxy, methylcarbonyloxy, ethylcarbonyloxy, dimethylaminocarbonyloxy, methylamino, ethylamino, dimethylamino, diethylamino, dipropylamino, benzylamino, or dibenzylamino; represents methyl substituted by the group —NR′R″ (where R′R″ together with the nitrogen represents pyrrolidine, piperidine, 4-trifluoromethylpiperidine, 3-trifluoromethylpiperidine, fluoromethylpiperidine, morpholine, dimethylmorpholine, piperazine, or N-methylpiperazine), acetyl, propionyl, n- or i-butyroyl, methoxy, ethoxy, n- or i-propoxy, n-, i-, s-, or t-butoxy, methoxycarbonyl, ethoxycarbonyl, n- or i-propoxycarbonyl, methoximinomethyl, ethoximinomethyl, cyclopropylmethoxyiminomethyl, benzyloxyiminomethyl, chlorobenzyloxyiminomethyl, methylcarbonyloxyiminomethyl, allyloxyiminomethyl, phenylsulphonylaminoiminomethyl, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s-, or t-butylamino, methylaminocarbonyl, ethylaminocarbonyl, n- or i-propylaminocarbonyl, dimethylamino, diethylamino, dimethylaminocarbonyl, acetylaminocarbonyl, propionylaminocarbonyl, n- or i-butyroylaminocarbonyl, N-methylacetylaminocarbonyl, N-methylpropionylaminocarbonyl, methoxycarbonylaminocarbonyl, ethoxycarbonylaminocarbonyl, n- or i-propoxycarbonylaminocarbonyl, N-methylmethoxycarbonylaminocarbonyl, or N-methylethoxycarbonylaminocarbonyl; represents methylaminocarbonylaminocarbonyl, ethylaminocarbonylaminocarbonyl, n- or i-propylamino-carbonylaminocarbonyl, N-methylmethylaminocarbonylaminocarbonyl, N-methyl-ethylaminocarbonylaminocarbonyl, N,O-dimethylhydroxylaminocarbonyl, N,O-diethylhydroxylaminocarbonyl, N-methyl-O-ethylhydroxylaminocarbonyl, or N-ethyl-O-methylhydroxylaminocarbonyl, optionally substituted by cyano; represents ethenyl, propenyl, butenyl, pentenyl, propenyloxy, butenyloxy, pentenyloxy, propenylamino, butenylamino, pentenylamino, allyloximinomethyl, ethynyl, propynyl, butynyl, pentynyl, hexynyl, heptynyl, propynyloxy, butynyloxy, pentynyloxy, propynylaminocarbonyl, butinylaminocarbonyl, or pentynylaminocarbonyl, in each case optionally substituted by cyano, hydroxy, methoxy, ethoxy, n- or i-propoxy, pyridyl (which is itself optionally substituted by halogen), thienyl, thiazolyl (which itself is optionally substituted by methyl or ethyl), trialkylsilyl, phenyl (which itself is optionally substituted by methyl, ethyl, methoxy, ethoxy, fluorine, chlorine, bromine, iodine, or trifluoromethyl), phenoxy, methoxycarbonyl, ethoxycarbonyl, fluorine, chlorine, or bromine; represents cyclopropyl, cyclopentyl, cyclohexyl, cyclopropyloxy, cyclopentyloxy, cyclohexyloxy, cyclopropylamino, cyclopentylamino, cyclohexylamino, cyclopropylmethyl, cyclopentylmethyl, cyclohexylmethyl, cyclopropylmethoxy, cyclopentylmethoxy, cyclohexylmethoxy, cyclopropylmethylamino, cyclopentylmethylamino, or cyclohexylmethylamino, in each case optionally substituted by cyano, fluorine, chlorine, bromine, iodine, methyl, ethyl, n- or i-propyl, or trifluoromethyl; represents dioxolan-2-yl, 1,3-dioxan-2-yl, oxazolyl, 1,2,4-oxadiazolyl, 1,2,4-thiadiazolyl, phenyl, phenoxy, phenylthio, phenylamino, benzyl, phenylethyl, phenylethynyl, phenylmethoxy, phenylethoxy, phenylmethylthio, phenylmethylamino, or phenylethylamino, in each case optionally substituted by nitro, amino, hydroxy, cyano, carbamoyl, thiocarbamoyl, fluorine, chlorine, bromine, iodine, methyl, ethyl, n- or i-propyl, n-, i-, s-, or t-butyl, trifluoromethyl, trichloromethyl, fluorodichloromethyl, chlorodifluoromethyl, methoxy, ethoxy, n- or i-propoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, methylthiomethyl, difluoromethylthio, trifluoromethylthio, chlorodifluoromethylthio, methylsulphinyl, ethylsulphinyl, trifluoromethylsulphinyl, methylsulphonyl, ethylsulphonyl, trifluoromethylsulphonyl, methylamino, ethylamino, n- or i-propylamino, dimethylamino, dimethylaminocarbonyl, dimethylaminosulphonyl, phenoxy, thienylsulphonylmethyl, piperidinomethyl, benzyl, or phenyl; or represents 2,4-dioxaspiro[5.5]undec-8-en-3-yl, 2,4-dioxaspiro[5.5]undecan-3-yl, or phenylethylamino.

26: An azinylimidazoazine of structure (IA), 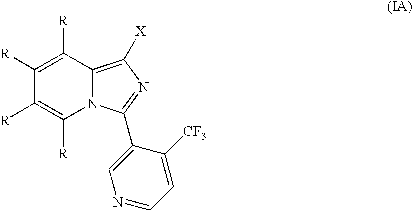
and/or salts and/or N-oxides thereof,
where
R represents in each case H (hydrogen), nitro, amino, cyano, fluorine, chlorine, bromine, or iodine; or represents methyl, ethyl, n- or i-propyl, n-, i-, s-, or t-butyl, methoxy, ethoxy, n- or i-propoxy, n-, i-, s-, or t-butoxy, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, methylamino, ethylamino, n- or i-propylamino, n-, i-, s-, or t-butylamino, dimethylamino, or diethylamino, in each case optionally substituted by cyano, fluorine, chlorine, bromine, methoxy, ethoxy, n- or i-propoxy, n-, i-, s-, or t-butoxy; or two neighboring R groups optionally together represent propane-1,3-diyl, butane-1,3-diyl, butane-1,4-diyl, pentane-1,3-diyl, pentane-1,4-diyl, or pentane-1,5-diyl or together with the azine group to which they are connected form a benzene ring, and
X represents H (hydrogen) or hydroxycarbonyl (COOH); represents aminocarbonyl optionally substituted by benzyloxycarbonyl, N,O-dimethylhydroxylaminocarbonyl, N,O-diethylhydroxylaminocarbonyl, N-methyl-O-ethylhydroxylaminocarbonyl, or N-ethyl-O-methylhydroxylaminocarbonyl; represents nitro, formyl, hydroximinomethyl, aminoiminomethyl, amino, cyano, fluorine, chlorine, bromine, or iodine; represents methyl, ethyl, n- or i-propyl, n-, i-, s-, or t-butyl, or n-pentyl, in each case optionally substituted by cyano, hydroxy, fluorine, chlorine, methoxy, ethoxy, n- or i-propoxy, methylcarbonyloxy, ethylcarbonyloxy, dimethylaminocarbonyloxy, methylamino, ethylamino, dimethylamino, diethylamino, dipropylamino, benzylamino, or dibenzylamino; represents methyl substituted by the group —NR′R″ (where R′R″ together with the nitrogen represents pyrrolidine, piperidine, 4-trifluoromethylpiperidine, 3-trifluoromethylpiperidine, fluoromethylpiperidine, morpholine, dimethylmorpholine, piperazine, or N-methylpiperazine), acetyl, propionyl, n- or i-butyroyl, methoxy, ethoxy, n- or i-propoxy, n-, i-, s-, or t-butoxy, methoxycarbonyl, ethoxycarbonyl, n- or i-propoxycarbonyl, methoximinomethyl, ethoximinomethyl, cyclopropylmethoxyiminomethyl, benzyloxyiminomethyl, chlorbenzyloxyiminomethyl, methylcarbonyloxyiminomethyl, allyloxyiminomethyl, phenylsulphonylaminoiminomethyl, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s-, or t-butylamino, methylaminocarbonyl, ethylaminocarbonyl, n- or i-propylaminocarbonyl, dimethylamino, diethylamino, dimethylaminocarbonyl, acetylaminocarbonyl, propionylaminocarbonyl, n- or i-butyroylaminocarbonyl, N-methylacetylaminocarbonyl, N-methylpropionylaminocarbonyl, methoxycarbonylaminocarbonyl, ethoxycarbonylaminocarbonyl, n- or i-propoxycarbonylaminocarbonyl, N-methylmethoxycarbonylaminocarbonyl, or N-methylethoxycarbonylaminocarbonyl; represents methylaminocarbonylaminocarbonyl, ethylaminocarbonylaminocarbonyl, n- or i-propylaminocarbonylaminocarbonyl, N-methylmethylaminocarbonylaminocarbonyl, N-methylethylaminocarbonylaminocarbonyl, N,O-dimethylhydroxylaminocarbonyl, N,O-diethylhydroxylaminocarbonyl, N-methyl-O-ethylhydroxylaminocarbonyl, or N-ethyl-O-methylhydroxylaminocarbonyl, optionally substituted by cyano; represents ethenyl, propenyl, butenyl, pentenyl, propenyloxy, butenyloxy, pentenyloxy, propenylamino, butenylamino, pentenylamino, allyloximinomethyl, ethynyl, propynyl, butynyl, pentynyl, hexynyl, heptynyl, propynyloxy, butynyloxy, pentynyloxy, propynylaminocarbonyl, butynylaminocarbonyl, or pentynylaminocarbonyl, in each case optionally substituted by cyano, hydroxy, methoxy, ethoxy, n- or i-propoxy, pyridyl (which is itself optionally substituted by halogen), thienyl, thiazolyl (which itself is optionally substituted by methyl or ethyl), trialkylsilyl, phenyl (which itself is optionally substituted by methyl, ethyl, methoxy, ethoxy, fluorine, chlorine, bromine, iodine, or trifluoromethyl), phenoxy, methoxycarbonyl, ethoxycarbonyl, fluorine, chlorine, or bromine; represents cyclopropyl, cyclopentyl, cyclohexyl, cyclopropyloxy, cyclopentyloxy, cyclohexyloxy, cyclopropylamino, cyclopentylamino, cyclohexylamino, cyclopropylmethyl, cyclopentylmethyl, cyclohexylmethyl, cyclopropylmethoxy, cyclopentylmethoxy, cyclohexylmethoxy, cyclopropylmethylamino, cyclopentylmethylamino, or cyclohexylmethylamino, in each case optionally substituted by cyano, fluorine, chlorine, bromine, iodine, methyl, ethyl, n- or i-propyl, or trifluoromethyl; represents dioxolan-2-yl, 1,3-dioxan-2-yl, oxazolyl, 1,2,4-oxadiazolyl, 1,2,4-thiadiazolyl, phenyl, phenoxy, phenylthio, phenylamino, benzyl, phenylethyl, phenylethynyl, phenylmethoxy, phenylethoxy, phenylmethylthio, phenylmethylamino, or phenylethylamino, in each case optionally substituted by nitro, amino, hydroxy, cyano, carbamoyl, thiocarbamoyl, fluorine, chlorine, bromine, iodine, methyl, ethyl, n- or i-propyl, n-, i-, s-, or t-butyl, trifluoromethyl, trichloromethyl, fluorodichloromethyl, chlorodifluoromethyl, methoxy, ethoxy, n- or i-propoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, methylthiomethyl, difluoromethylthio, trifluoromethylthio, chlordifluoromethylthio, methylsulphinyl, ethylsulphinyl, trifluoromethylsulphinyl, methylsulphonyl, ethylsulphonyl, trifluoromethylsulphonyl, methylamino, ethylamino, n- or i-propylamino, dimethylamino, dimethylaminocarbonyl, dimethylaminosulphonyl, phenoxy, thienylsulphonylmethyl, piperidinomethyl, benzyl, or phenyl; or represents 2,4-dioxaspiro[5.5]undec-8-en-3-yl, 2,4-dioxaspiro[5.5]undecan-3-yl, or phenylethylamino.

27: An azinylimidazoazine of structure (IB) 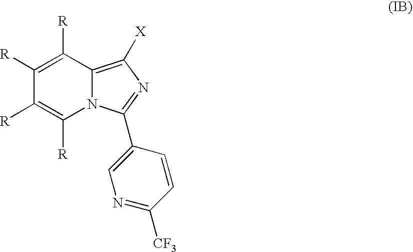
and/or salts and/or N-oxides thereof,
where
R represents in each case H (hydrogen), nitro, amino, cyano, fluorine, chlorine, bromine, or iodine; or represents methyl, ethyl, n- or i-propyl, n-, i-, s-, or t-butyl, methoxy, ethoxy, n- or i-propoxy, n-, i-, s-, or t-butoxy, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, methylamino, ethylamino, n- or i-propylamino, n-, i-, s-, or t-butylamino, dimethylamino, or diethylamino, in each case optionally substituted by cyano, fluorine, chlorine, bromine, methoxy, ethoxy, n- or i-propoxy, n-, i-, s- or t-butoxy; or two neighboring R groups optionally together represent propane-1,3-diyl, butane-1,3-diyl, butane-1,4-diyl, pentane-1,3-diyl, pentane-1,4-diyl, or pentane-1,5-diyl or together with the azine group to which they are connected form a benzene ring, and
X represents H (hydrogen) or hydroxycarbonyl (COOH); represents aminocarbonyl optionally substituted by benzyloxycarbonyl, N,O-dimethylhydroxylaminocarbonyl, N,O-diethylhydroxylaminocarbonyl, N-methyl-O-ethylhydroxylaminocarbonyl, or N-ethyl-O-methylhydroxylaminocarbonyl; represents nitro, formyl, hydroximinomethyl, aminoiminomethyl, amino, cyano, fluorine, chlorine, bromine, or iodine; represents methyl, ethyl, n- or i-propyl, n-, i-, s-, or t-butyl, or n-pentyl, in each case optionally substituted by cyano, hydroxy, fluorine, chlorine, methoxy, ethoxy, n- or i-propoxy, methylcarbonyloxy, ethylcarbonyloxy, dimethylaminocarbonyloxy, methylamino, ethylamino, dimethylamino, diethylamino, dipropylamino, benzylamino, or dibenzylamino; represents methyl substituted by the group —NR′R″ (where R′R″ together with the nitrogen atom represents pyrrolidine, piperidine, 4-trifluoromethylpiperidine, 3-trifluoromethylpiperidine, fluoromethylpiperidine, morpholine, dimethylmorpholine, piperazine, or N-methylpiperazine), acetyl, propionyl, n- or i-butyroyl, methoxy, ethoxy, n- or i-propoxy, n-, i-, s-, or t-butoxy, methoxycarbonyl, ethoxycarbonyl, n- or i-propoxycarbonyl, methoximinomethyl, ethoximinomethyl, cyclopropylmethoxyiminomethyl, benzyloxyiminomethyl, chlorobenzyloxyiminomethyl, methylcarbonyloxyiminomethyl, allyloxyiminomethyl, phenylsulphonylaminoiminomethyl, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, n- or i-propylsulphonyl, methylamino, ethylamino, n- or i-propylamino, n-, i-, s-, or t-butylamino, methylaminocarbonyl, ethylaminocarbonyl, n- or i-propylaminocarbonyl, dimethylamino, diethylamino, dimethylaminocarbonyl, acetylaminocarbonyl, propionylaminocarbonyl, n- or i-butyroylaminocarbonyl, N-methylacetylaminocarbonyl, N-methylpropionylaminocarbonyl, methoxycarbonylaminocarbonyl, ethoxycarbonylaminocarbonyl, n- or i-propoxycarbonylaminocarbonyl, N-methylmethoxycarbonylaminocarbonyl, or N-methylethoxycarbonylaminocarbonyl; represents methylaminocarbonylaminocarbonyl, ethylaminocarbonylaminocarbonyl, n- or i-propylaminocarbonylaminocarbonyl, N-methylmethylaminocarbonylaminocarbonyl, N-methylethylaminocarbonylaminocarbonyl, N,O-dimethylhydroxylaminocarbonyl, N,O-diethylhydroxylaminocarbonyl, N-methyl-O-ethylhydroxylaminocarbonyl, N-ethyl-O-methylhydroxylaminocarbonyl, optionally substituted by cyano; represents ethenyl, propenyl, butenyl, pentenyl, propenyloxy, butenyloxy, pentenyloxy, propenylamino, butenylamino, pentenylamino, allyloximinomethyl, ethynyl, propynyl, butynyl, pentynyl, hexynyl, heptynyl, propynyloxy, butynyloxy, pentynyloxy, propynylaminocarbonyl, butynylaminocarbonyl, or pentynylaminocarbonyl, in each case optionally substituted by cyano, hydroxy, methoxy, ethoxy, n- or i-propoxy, pyridyl (which is optionally itself substituted by halogen), thienyl, thiazolyl (which itself is optionally substituted by methyl or ethyl), trialkylsilyl, phenyl (which itself is optionally substituted by methyl, ethyl, methoxy, ethoxy, fluorine, chlorine, bromine, iodine, or trifluoromethyl), phenoxy, methoxycarbonyl, ethoxycarbonyl, fluorine, chlorine, or bromine; represents cyclopropyl, cyclopentyl, cyclohexyl, cyclopropyloxy, cyclopentyloxy, cyclohexyloxy, cyclopropylamino, cyclopentylamino, cyclohexylamino, cyclopropylmethyl, cyclopentylmethyl, cyclohexylmethyl, cyclopropylmethoxy, cyclopentylmethoxy, cyclohexylmethoxy, cyclopropylmethylamino, cyclopentylmethylamino, or cyclohexylmethylamino, in each case optionally substituted by cyano, fluorine, chlorine, bromine, iodine, methyl, ethyl, n- or i-propyl, or trifluoromethyl; represents dioxolan-2-yl, 1,3-dioxan-2-yl, oxazolyl, 1,2,4-oxadiazolyl, 1,2,4-thiadiazolyl, phenyl, phenoxy, phenylthio, phenylamino, benzyl, phenylethyl, phenylethynyl, phenylmethoxy, phenylethoxy, phenylmethylthio, phenylmethylamino, or phenylethylamino, in each case optionally substituted by nitro, amino, hydroxy, cyano, carbamoyl, thiocarbamoyl, fluorine, chlorine, bromine, iodine, methyl, ethyl, n- or i-propyl, n-, i-, s-, or t-butyl, trifluoromethyl, trichloromethyl, fluorodichloromethyl, chlorodifluoromethyl, methoxy, ethoxy, n- or i-propoxy, difluoromethoxy, trifluoromethoxy, chlorodifluoromethoxy, fluoroethoxy, chloroethoxy, difluoroethoxy, dichloroethoxy, trifluoroethoxy, methylthio, ethylthio, n- or i-propylthio, methylthiomethyl, difluoromethylthio, trifluoromethylthio, chlorodifluoromethylthio, methylsulphinyl, ethylsulphinyl, trifluoromethylsulphinyl, methylsulphonyl, ethylsulphonyl, trifluoromethylsulphonyl, methylamino, ethylamino, n- or i-propylamino, dimethylamino, dimethylaminocarbonyl, dimethylaminosulphonyl, phenoxy, thienylsulphonylmethyl, piperidinomethyl, benzyl, or phenyl; or represents 2,4-dioxaspiro[5.5]undec-8-en-3-yl, 2,4-dioxaspiro[5.5]undecan-3-yl, or phenylethylamino.

28: A method for the preparation of azinylimidazoazines of structure (I) according to claim 22 comprising reacting an N-azinylalkylazine carboxamide of structure (II) 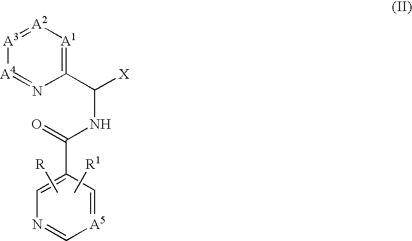
in which
A¹, A², A³, A⁴, and A⁵ are the same or different and in each case represent N (nitrogen) or the group C—R, with the provisos that the heterocycle containing the substituents A¹, A², A³, and A⁴ must contain 1 to 4 N atoms and no more than two N atoms are next to each other, and
R, R¹, and X have the meaning given for structure (I) in claim 22,
with a condensation agent, optionally in the presence of one or more diluents, and optionally transforming the resultant compounds of structure (I) to other compounds of structure (I).

29: An N-azinylalkylazine carboxamide of structure (II) 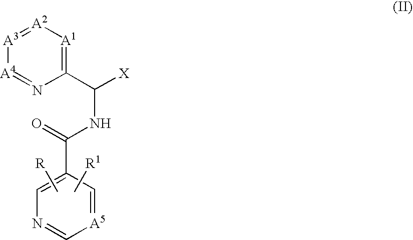
and/or salts and/or N-oxides thereof,
in which
A¹, A², A³, A⁴, and A⁵ are the same or different and in each case represent N (nitrogen) or the group C—R, with the provisos that the heterocycle containing the substituents A¹, A², A³, and A⁴ must contain 1 to 4 N atoms and no more than two N atoms are next to each other,
R in each case independently represents H (hydrogen), nitro, amino, cyano, or halogen; or represents optionally substituted alkyl, alkoxy, alkylthio, alkylsulphinyl, alkylsulphonyl, alkylamino, or dialkylamino; or two neighboring R groups optionally together represent alkanediyl or together with the azine group to which they are connected form a benzene ring,
R¹ represents (C₁-C₄)-haloalkyl, and
X represents H (hydrogen), nitro, formyl, hydroximinomethyl (—CH═N—OH), aminoiminomethyl (—CH═N—NH2), amino, cyano, or halogen; or represents optionally substituted —COOH, aminocarbonyl (—CO—NH₂), alkyl, alkylcarbonyl, alkoxy, alkoxycarbonyl, alkoximinomethyl (—CH═N—O-alkyl), alkylaminoiminomethyl (—CH═N—NH-alkyl), dialkylaminoiminomethyl, cycloalkylalkoxyiminomethyl, benzyloxyiminomethyl, alkenyloxyiminomethyl, arylsulphonylaminoiminomethyl, alkylcarbonyloxyiminomethyl, alkylthio, alkylsulphinyl, alkylsulphonyl, alkylamino, aminocarbonyl, hydroxycarbonyl alkylaminocarbonyl, alkenylaminocarbonyl, alkynylaminocarbonyl, dialkylamino, dialkylaminocarbonyl, alkylcarbonylaminocarbonyl, N-alkylalkylcarbonylaminocarbonyl, alkoxycarbonylaminocarbonyl, N-alkylalkoxycarbonylaminocarbonyl, alkylaminocarbonylaminocarbonyl, N-alkyl-N-alkylaminocarbonylaminocarbonyl, alkenyl, alkenyloxy, alkenylamino, alkenyloximinomethyl, alkynyl, alkynyloxy, alkynylamino, cycloalkyl, cycloalkyloxy, cycloalkylalkoximinomethyl, cycloalkylamino, cycloalkylalkyl, cycloalkylalkoxy, cycloalkylalkylamino, aryl, aryloxy, arylthio, arylamino, arylaminoiminomethyl, arylalkyl, arylethynyl, arylalkoxy, arylalkylthio, arylalkylamino, arylalkylaminoiminomethyl, arylalkoxyiminomethyl, arylsulphonylaminoiminomethyl, heterocyclyl, heterocyclyloxy, heterocyclylthio, heterocyclylamino, heterocyclylalkyl, heterocyclylalkynyl, heterocyclylalkoxy, heterocyclylalkylthio, or heterocyclylalkylamino,
but with the exclusion of the compounds N-(2-pyridinylmethyl)-4-trifluoromethylpyridine-3-carboxamide, 4-trifluoromethyl-N-[(5-trifluoromethyl-2-pyridinyl)methyl]-pyridine-3-carboxamide, 4-trifluoromethyl-N-[(2,6-dichloro-4-pyridinyl)methyl]-pyridine-3-carboxamide, 4-trifluoromethyl-N-[(6-chloro-2-pyridinyl)methyl]pyridine-3-carboxamide, 4-trifluoromethyl-N-[(2,3,5,6-tetrachloro-4-pyridinyl)methyl]pyridine-3-carboxamide, 4-trifluoromethyl-N-[(2-pyridinyl)methyl]pyridine-3-carboxamide, 4-trifluoromethyl-N-[(3-trifluoromethyl-2-pyridinyl)methyl]pyridine-3-carboxamide, 4-trifluoromethyl-N-[(5,6-dichloro-3-pyridinyl)methyl]pyridine-3-carboxamide, 4-trifluoromethyl-N-[(6-chloro-3-pyridinyl)methyl]pyridine-3-carboxamide, 4-trifluoromethyl-N-[(2-chloro-3-pyridinyl)methyl]pyridine-3-carboxamide, 4-trifluoromethyl-N-[(3-quinolinyl)methyl]pyridine-3-carboxamide, 4-trifluoromethyl-N-[(6-trifluoromethyl-2-pyridinyl)methyl]pyridine-3-carboxamide, N-[(2-pyrazinyl)-methyl]-4-trifluoromethylpyridine-3-carboxamide, N-[(3-chloro-5-trifluoromethyl-2-pyridinyl)methyl]-4-trifluoromethylpyridine-3-carboxamide, 2-bromo-6-trifluoromethyl-N-[(3-chloro-5-trifluoromethyl-2-pyridinyl)methyl]pyridine-3-carboxamide, N-[(3-chloro-5-trifluoromethyl-2-pyridinyl)methyl]-2-methyl-6-trifluoromethylpyridine-3-carboxamide, N-[(3-chloro-5-trifluoromethyl-2-pyridinyl)methyl]-2-methoxy-6-trifluoromethylpyridine-3-carboxamide, N-[(3-chloro-5-trifluoromethyl-2-pyridinyl)methyl]-2-methoxymethyl-6-trifluoromethylpyridine-3-carboxamide, N-[3-chloro-5-trifluoromethyl-2-pyridinyl)methyl]-6-trifluoromethylpyridine-3-carboxamide, and N-[[3-chloro-5-(trifluoromethyl)pyridin-2-yl](piperidin-1-yl)methyl]-4-(trifluoromethyl)nicotinamide.

30: A compounds of structure (II-b) 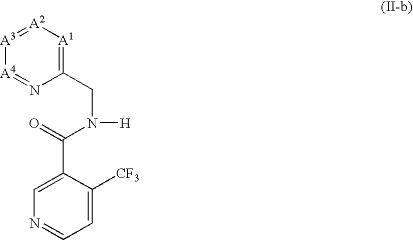
and/or salts and/or N-oxides thereof,
where
A¹, A², A³, and A⁴ are the same or different and in each case represent N (nitrogen) or the group C—R, with the provisos that the heterocycle containing the substituents A¹, A², A³, and A⁴ must contain 2 or 3 N atoms and no more than two N atoms are next to each other, and
R represents H (hydrogen), nitro, amino, cyano, or halogen; or represents optionally substituted alkyl, alkoxy, alkylthio, alkylsulphinyl, alkylsulphonyl, alkylamino, or dialkylamino; or two neighboring R groups optionally together represent alkanediyl or together with the azine group to which they are connected form a benzene ring,
except for compounds in which the heterocycle containing the substituents A¹, A², A³, and A⁴ represents unsubstituted pyrazinyl.

31: A compound of structure (II-b) according to claim 30 where
A¹, A², A³ and A⁴ are the same or different and in each case represent N (nitrogen) or the group C—R, with the provisos that the heterocycle containing the substituents A¹, A², A³, and A⁴ must contain 2 or 3 N atoms and no more than two N atoms are next to each other, and
R represents in each case H (hydrogen), nitro, amino, cyano, or halogen; or represents alkyl, alkoxy, alkylthio, alkylsulphinyl, alkylsulphonyl, alkylamino, or dialkylamino having in each case 1 to 6 carbon atoms in the alkyl groups and being in each case optionally substituted by cyano, halogen, or C₁-C₄-alkoxy; or two neighboring R groups optionally together represent alkanediyl with 3 to 5 carbon atoms or together with the azine group to which they are connected form a benzene ring.

32: A compound of structure (II-b) according to claim 30 where
A¹, A², A³ and A⁴ are the same or different and in each case represent N (nitrogen) or the group C—R, with the provisos that the heterocycle containing the substituents A¹, A², A³, and A⁴ must contain 2 or 3 N atoms and no more than two N atoms are next to each other, and
R in each case represents H (hydrogen), nitro, amino, cyano, fluorine, chlorine, bromine, or iodine; or represents methyl, ethyl, n- or i-propyl, n-, i-, s-, or t-butyl, methoxy, ethoxy, n- or i-propoxy, n-, i-, s-, or t-butoxy, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, methylamino, ethylamino, n- or i-propylamino, n-, i-, s-, or t-butylamino, dimethylamino, or diethylamino, in each case optionally substituted by cyano, fluorine, chlorine, bromine, methoxy, ethoxy, n- or i-propoxy, or n-, i-, s-, or t-butoxy; or two neighboring R groups optionally together represent propane-1,3-diyl, butane-1,3-diyl, butane-1,4-diyl, pentane-1,3-diyl, pentane-1,4-diyl, or pentane-1,5-diyl or together with the azine group to which they are connected form a benzene ring.

33: A compound of structure (II-b) according to claim 30 where
A¹, A², A³ and A⁴ are the same or different and in each case represent N (nitrogen) or the group C—R, with the provisos that the heterocycle containing the substituents A¹, A², A³, and A⁴ contains 2 N atoms, and
R in each case represents H (hydrogen), nitro, amino, cyano, fluorine, chlorine, bromine, or iodine; or represents methyl, ethyl, n- or i-propyl, n-, i-, s-, or t-butyl, methoxy, ethoxy, n- or i-propoxy, n-, i-, s-, or t-butoxy, methylthio, ethylthio, n- or i-propylthio, n-, i-, s-, or t-butylthio, methylsulphinyl, ethylsulphinyl, n- or i-propylsulphinyl, methylsulphonyl, ethylsulphonyl, methylamino, ethylamino, n- or i-propylamino, n-, i-, s-, or t-butylamino, dimethylamino, or diethylamino, in each case optionally substituted by cyano, fluorine, chlorine, bromine, methoxy, ethoxy, n- or i-propoxy, or n-, i-, s-, or t-butoxy; or two neighboring R groups optionally together represent propane-1,3-diyl, butane-1,3-diyl, butane-1,4-diyl, pentane-1,3-diyl, pentane-1,4-diyl, or pentane-1,5-diyl or together with the azine group to which they are connected form a benzene ring.

34: A method for the preparation of azinylalkylazine carboxamides of structure (II) according to claim 29 comprising reacting an azine carbonyl halide of structure (III) 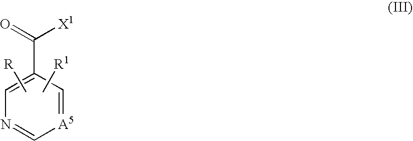
in which
A⁵, R, and R¹ have the meanings given for formula (II) in claim 29, and
X¹ represents halogen,
with an azinyl alkylamine of structure (IV) 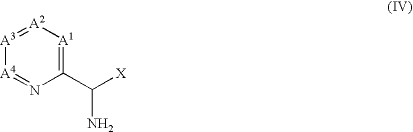
in which A¹, A², A³, A⁴, and X have the meanings given for formula (II) of claim 29,
optionally in the presence of a diluent and optionally in the present of a reaction auxiliary.

35: A plant treatment or pest control agent comprising one or more compounds of structure (I) according to claim 22 and one or more auxiliaries and additives.

36: A plant treatment or pest control agent comprising one or more compounds of structure (II) according to claim 29 and one or more auxiliaries and additives.

37: A plant treatment or pest control agent comprising one or more compounds of structure (II-b) according to claim 30 and one or more auxiliaries and additives.

38: A method for the control of undesirable micro-organisms in and/or on plants and/or pests comprising bringing the micro-organisms or pests into direct or indirect contact with a compound of structure (I) according to claim 22.

39: A method for the control of undesirable micro-organisms in and/or on plants and/or zoopests comprising bringing the micro-organisms or zoopests into direct or indirect contact with an agent according to claim 35.

40: A method for the control of undesirable micro-organisms in and/or on plants and/or zoopests comprising bringing the micro-organisms or zoopests into direct or indirect contact with an agent according to claim 36.

41: A method according to claim 38 wherein the zoopest is a veterinary pest.

42: A method according to claim 39 wherein the zoopest is a veterinary pest.

43: A method according to claim 40 wherein the zoopest is a veterinary pest.
